(19) United States
(12) Patent Application Publication
(10) Pub. No.: US 2018/0195091 A1
(43) Pub. Date: Jul. 12, 2018

(54) THERMOPHILIC MICROORGANISMS FOR CONVERSION OF LIGNOCELLULOSIC BIOMASS TO ETHANOL

(71) Applicant: THE TRUSTEES OF DARTMOUTH COLLEGE, Hanover, NH (US)

(72) Inventors: Daniel G. Olson, Hanover, NH (US); Lee R. Lynd, Plainfield, NH (US); Liang TIAN, Bethesda, MD (US); Jonathan LO, Norwich, VT (US); Jilai ZHOU, West Lebanon, NH (US); Shuen HON, West Lebanon, NH (US); Tianyong ZHENG, Hanover, NH (US)

(21) Appl. No.: 15/746,379

(22) PCT Filed: Jul. 23, 2016
(86) PCT No.: PCT/US16/43770

§ 371 Date: Jan. 19, 2018

Related U.S. Application Data
(60) Provisional application No. 62/196,051, filed on Jul. 23, 2015.

(51) Int. Cl. C12P 7/10; C12N 1/20; C12N 15/74; C12R 1/145

(52) U.S. Cl. CPC C12P 7/10; C12N 1/20; C12N 15/74; C12Y 102/0101; C12Y 101/01001; C12Y 102/01005; C12R 1/145

(57) ABSTRACT

It is disclosed here engineered cellulolytic microorganisms capable of producing ethanol from lignocellulosic feedstock with high yield. Multiple genes in Thermoanaerobacterium saccharolyticum that are involved in the pyruvate to ethanol pathway are disclosed which may be transferred into C. thermocellum or other natively cellulolytic microorganisms.

### CROSS-REFERENCE TO RELATED APPLICATION

This application claims priority to U.S. Patent Application No. 62/196,051 filed Jul. 23, 2015, the entire content of which is hereby incorporated by reference into this application.

### GOVERNMENT INTERESTS

This invention was made with government support under Award No. DE-AC05-00OR-22725 awarded by the BioEnergy Science Center (BESC) under the Department of Energy. The government has certain rights in this invention.

### BACKGROUND

### I. Field of the Invention

The disclosure relates to conversion of biomass to biofuel or other useful products. More particularly, the disclosure pertains to the generation of microorganisms having higher ethanol yields.

### II. Description of the Related Art

Thermophilic bacteria have been engineered to produce ethanol from the cellulose and/or hemicellulose fractions of biomass. Examples of such thermophilic bacteria include Clostridium thermocellum and Thermoanaerobacterium saccharolyticum, among others.

Thermoanaerobacterium saccharolyticum is a thermophilic anaerobe that ferments xylan and other sugars derived from biomass. It has been engineered to produce ethanol at high yield and titer. However, the genes involved in the pathway for pyruvate-to-ethanol conversion in T. saccharolyticum are either unknown or poorly characterized.

Clostridium thermocellum is a cellulolytic microorganism capable of producing ethanol from lignocellulosic feedstock. C. thermocellum is a gram-positive obligate anaerobe that rapidly consumes cellulose. However, engineered strains of C. thermocellum typically produce ethanol at relatively low yields (50% of theoretical maximum).

### SUMMARY

The presently disclosed instrumentalities advance the art by providing engineered strains of thermophilic bacteria capable of producing ethanol from lignocellulosic feedstock with high yield. In one embodiment, this disclosure provides characterization of several genes in Thermoanaerobacterium saccharolyticum that are involved in the pyruvate to ethanol pathway. In another embodiment, these genes may be transferred into C. thermocellum or other natively cellulolytic microorganisms to create thermophilic bacteria capable of producing ethanol from lignocellulosic feedstock with high yield.

C. thermocellum is able to rapidly solubilize cellulosic biomass and convert glucan derivatives thereof to pyruvate and reduced nicotinamide electron carriers (i.e. NADH or NADPH). Wild-type strains of C. thermocellum convert pyruvate and reduced nicotinamide electron carriers to acetic acid, ethanol, lactic acid, formic acid, hydrogen, and CO₂. In one embodiment, genes encoding key enzymes from the ethanol production pathway of T. saccharolyticum may be transferred to C. thermocellum, which may enable engineered strains of C. thermocellum to achieve high ethanol yield, with minimal formation of undesirable co-products.

In one embodiment, the pathway of T. saccharolyticum may involve six genes: pyruvate-ferredoxin oxidoreductase (pfor), ferredoxin, nfnA, nfnB, mutated bifunctional aldehyde and alcohol dehydrogenase E (adhE), and alcohol dehydrogenase A (adhA). Proteins coded for by these genes mediate 4 Reactions as shown below.

$$\begin{matrix}Pyuvate + CoA - SH + Fd ( ox ) {}^{pfor} Acetyl - CoA + {CO}_{2} + {Fd}_{( red )} & 1 \\ {Fd}_{red} + NADH + 2 NADPH {}^{nfnA , nfnB} 2 NADPH + {Fd}_{( ox )} + NAD & 2 \\ Acetyl - CoA + NADPH {}^{{adhE}^{m}} Acetaldehyde + NADP & 3 \\ Acetaldehyde + NADPH {}^{adhA} Ethanol + NADP & 4\end{matrix}$$

The stoichiometry of converting glucose (from cellulose) to pyruvate operative in C. thermocellum is:

$$1 / 2 Glucose + NAD + ADP {}^{glycolytic enzymes} Pyruvate + NADH + ATP \qquad 5$$

The sum of Reactions 1 through 5 above achieves stoichiometric conversion of glucose to ethanol:

$$1 / 2 Glucose + ADP {}^{glycolytic enzymes , pfor , ferredoxin , nfnA , nfnB , {adhE}^{m} , adhA} Ethanol + ATP \qquad 6$$

The above abbreviations will be readily understood by practitioners in the field. It is to be noted that C. thermocellum may produce some GTP in lieu of ATP. In another aspect, the stoichiometry of Reaction 6 may sometimes need to be modified due to synthesis in the cells.

In one embodiment, all 6 genes of T. saccharolyticum, namely, pyruvate-ferredoxin oxidoreductase (pfor), ferredoxin, nfnA, nfnB, mutated bifunctional aldehyde and alcohol dehydrogenase E (adhE), and alcohol dehydrogenase A (adhA), may be transferred into the bacterium Clostridium thermocellum. In another aspect, only some but not all of these 6 genes are transferred into the bacterium C. thermocellum. In another aspect, only the adhE and adhA genes are transferred into the bacterium C. thermocellum.

In another embodiment, the engineered C. thermocellum strain may produce ethanol at high yield in a pathway involving pyruvate conversion via pyruvate ferredoxin oxidoreductase, which is in contrast to the use of pyruvate decarboxylase in yeast, Zymomonas mobilis, and engineered strains of Escherichia coli.

In another embodiment, the engineered C. thermocellum strain of this disclosure utilizes NADPH as the electron donor for the 2-step reduction of Acetyl-CoA to ethanol. In another embodiment, another point of novelty of the engineered C. thermocellum strain is the production of NADPH from NADH and reduced ferredoxin via the NFN reaction.

In one embodiment, a cellulolytic microorganism having a modified pyruvate-to-ethanol pathway may be generated. In one aspect, the microorganism may contain an adhE gene and an adhA gene encoding an aldehyde and alcohol dehydrogenase E and an alcohol dehydrogenase A, respectively. In another aspect, the aldehyde and alcohol dehydrogenase E (AdhE) may have a sequence that is at least 90% identical to the sequence of SEQ ID No. 1:

(SEQ ID No. 1)
MATTKTELDVQKQIDLLVSRAQEAQKKFMSYTQEQIDAIVKAMALAGVDK
HVELAKMAYEETKMGVYEDKITKNLFATEYVYHDIKNEKTVGIINENIEE
NYMEVAEPIGVIAGVTPVTNPTSTTMFKCLISIKTRNPIIFSFHPKAIKC
SIAAAKVMYEAALKAGAPEGCIGWIETPSIEATQLLMTHPGVSLILATGG
AGMVKAAYSSGKPALGVGPGNVPCYIEKSANIKRAVSDLILSKTFDNGVI
CASEQAVIIDEEIADEVKKLMKEYGCYFLNKDEIKKLEKFAIDEQSCAMS
PAVVGQPAAKIAEMAGFKVPEGTKILVAEYEGVGPKYPLSREKLSPILAC
YTVKDYNEGIKKCEEMTEFGGLGHSAVIHSENQNVINEFARRVRTGRLIV
NSPSSQGAIGDIYNTNTPSLTLGCGSMGRNSTTDNVSVKNLLNIKRVVIR
KDRMKWFKIPPKIYFESGSLQYLCKVKRKKAFIVTDPFMVKLGFVDKVTY
QLDKANIEYEIFSEVEPDPSVDTVMNGVKIMNSYNPDLIIAVGGGSAIDA
AKGMWLFYEYPDTEFETLRLKFADIRKRAFKFPELGKKALFIAIPTTSGT
GSEVTAFAVITDKKRNIKYPLADYELTPDIAIIDPDLTKTVPPSVTADTG
MDVLTHAIEAYVSVMASDYTDALAEKAIKIVFEYLPRAYKNGNDEEAREK
MHNASCMAGMAFTNAFLGINHSMAHILGGKFHIPHGRANAILLPYVIRYN
AEKPTKFVAFPQYEYPKAAERYAEIAKFLGLPASTVEEGVESLIEAIKNL
MKELNIPLTLKDAGINKEQFEKEIEEMSDIAFNDQCTGTNPRMPLTKEIA
EIYRKAYGA.

In another aspect, the alcohol dehydrogenase A (AdhA) may have a sequence that is at least 90% identical to the sequence of SEQ ID No. 2:

(SEQ ID No. 2)
MWETKVNPSKIFELRCKNTTYFGVGSIHKIKDILENLKINGINNVIFITG
KGSYKTSGAWDVVRPVLEELDLKYSLYDKVGPNPTVDMIDEAAKIGRESG
AKAVIGIGGGSPIDTAKSVAVLLKYTDKNARELYKQKFIPDDAVPIIAIN
LTHGTGTEVDRFAVATIPEKNYKPAIAYDCLYPMFAIDDPSLMTKLDKKQ
TIAVTVDALNHITEAATTLVASPYSILTAKETVRLIVRYLPAAVNDPLNI
VARYYLLYASALAGISFDNGLLHLTHALEHPLSAVKPEIAHGLGLGAILP
AVIKAIYPATAEVLADVYSPIVPGLKGLPVEAEYVAEKVQEWLFSVGCIQ
KLSDFGFTKDDIPNLVKLAKTTPSLDGLLSIAPVEATESVIEKIYLKSL.

In one aspect, the microorganism is a natively cellulolytic microorganism. In another aspect, the microorganism is a thermophilic bacterium. In another aspect, the microorganism is a transgenic microorganism. For example, the microorganism may be a transgenic Clostridium thermocellum.

In another embodiment, the sequence of the aldehyde and alcohol dehydrogenase E encoded by the adhE gene in the transgenic microorganism is at least 95%, 98%, 99%, 99.9%, or 100% identical to the sequence of SEQ ID No. 1.

In another embodiment, the modified microorganism may have an endogenous adhE gene so only an exogenous adhA gene is introduced into the modified microorganism through transgenic technology. In another embodiment, the sequence of the alcohol dehydrogenase A encoded by the adhA gene in the transgenic microorganism is at least 95%, 98%, 99%, 99.9%, or 100% identical to the sequence of SEQ ID No. 2.

In another embodiment, the aldehyde and alcohol dehydrogenase E encoded by the adhE gene may have a sequence of SEQ ID No. 3. This T. saccharolyticum AdhE has a G544D mutation and may be transferred into C. thermocellum.

(SEQ ID No. 3)
MATTKTELDVQKQIDLLVSRAQEAQKKFMSYTQEQIDAIVKAMALAGVDK
HVELAKMAYEETKMGVYEDKITKNLFATEYVYHDIKNEKTVGIINENIEE
NYMEVAEPIGVIAGVTPVTNPTSTTMFKCLISIKTRNPIIFSFHPKAIKC
SIAAAKVMYEAALKAGAPEGCIGWIETPSIEATQLLMTHPGVSLILATGG
AGMVKAAYSSGKPALGVGPGNVPCYIEKSANIKRAVSDLILSKTFDNGVI
CASEQAVIIDEEIADEVKKLMKEYGCYFLNKDEIKKLEKFAIDEQSCAMS
PAVVGQPAAKIAEMAGFKVPEGTKILVAEYEGVGPKYPLSREKLSPILAC
YTVKDYNEGIKKCEEMTEFGGLGHSAVIHSENQNVINEFARRVRTGRLIV
NSPSSQGAIGDIYNTNTPSLTLGCGSMGRNSTTDNVSVKNLLNIKRVVIR
KDRMKWFKIPPKIYFESGSLQYLCKVKRKKAFIVTDPFMVKLGFVDKVTY
QLDKANIEYEIFSEVEPDPSVDTVMNGVKIMNSYNPDLIIAVGDGSAIDA
AKGMWLFYEYPDTEFETLRLKFADIRKRAFKFPELGKKALFIAIPTTSGT
GSEVTAFAVITDKKRNIKYPLADYELTPDIAIIDPDLTKTVPPSVTADTG
MDVLTHAIEAYVSVMASDYTDALAEKAIKIVFEYLPRAYKNGNDEEAREK
MHNASCMAGMAFTNAFLGINHSMAHILGGKFHIPHGRANAILLPYVIRYN
AEKPTKFVAFPQYEYPKAAERYAEIAKFLGLPASTVEEGVESLIEAIKNL
MKELNIPLTLKDAGINKEQFEKEIEEMSDIAFNDQCTGTNPRMPLTKEIA
EIYRKAYGA.

In another embodiment, the microorganism may also contain either or both of nfnA gene and nfnB gene from Thermoanaerobacterium saccharolyticum. The sequences of the nfnA and nfnB genes from Thermoanaerobacterium saccharolyticum are SEQ ID. No. 4 and SEQ ID. No. 5, respectively, as shown below:

(SEQ ID No. 4)
MNEILEKKQLNPTVKMMVINAPLMAKKAKPGQFVIVRVDEKGERIPLTIA
DYDRNKGTITIIFQEVGMSTKKLGTLNVGDRLHDFVGPLGKPVEFSKDTK
RVLAIGGGVGVAPLYPKVKMLNEMKVPVDSIIGGRSAEYVILEDEMKKVS
ENLYITTDDGTKGRKGFVTDVLKELIEKDNKYDEVIAIGPLIMMKMVCNI
TKEYNIPTMVSMNPIMIDGTGMCGGCRVTVGGETKFACVDGPAFDGLKVD
FDEAMRRQNMYKDMERKVLENYEHECKLGGILNG
(SEQ ID No. 5)
MANMSLKKVPMPEQEPDQRNKNFKEVALGYEENMAVEEAERCIQCKNQPC
VEGCPVHVKIPEFIKLIANRDFEGAYQKIKETNNLPAICGRVCPQESQCE
SVCTRGKKGEPVAIGRLERFTADWHMKNNEDKIEKPETNGRKVAVIGSGP
AGLSCAGDLAKMGYDTTIFEAFHTPGGVLMYGIPEFRLPKEIVQKEIDSL
KKLGVKIETNMVIGKILTIDDLFDMGYEAVFIGTGAGLPKFMNIPGENLN
GVYSANEFLTRINLMKAYDFPNSPTPVKVGKKVAVVGGGNVAMDAARSAK
RMGAEEVYIVYRRSEEEMPARLEEIHHAKEEGIIFKLLTNPVRIIGDESG
SVKGIECVNMVLGDVDESGRRRPVEEKGSEHVIDVDTVIIAIGQSPNPLI
TSTTEGLEKQRWGGIIVNEETLETSRRGVFAGGDAVTGAATVILAMGAGK
KAAASIHKYLSEK

In another embodiment, the microorganism may also contain one or both of nfnA gene and nfnB gene from Thermoanaerobacterium saccharolyticum. In one aspect, one or both of nfnA gene and nfnB gene from Thermoanaerobacterium saccharolyticum may be introduced into Clostridium thermocellum. In another aspect, the one or both of nfnA gene and nfnB gene from Thermoanaerobacterium saccharolyticum may be modified before being introduced into Clostridium thermocellum. In another aspect, the modified nfnA gene and/or nfnB gene may be at least 90%, 95%, 99% or 100% identical to SEQ ID No. 4 and No. 5, respectively.

In another embodiment, only exogenous adhA and/or adhE genes are introduced into the cellulolytic microorganism, but no exogenous nfnA or exogenous nfnB gene is introduced into the microorganism.

In another embodiment, the microorganism may also contain the ferredoxin gene from Thermoanaerobacterium saccharolyticum. The sequence of the ferredoxin gene from Thermoanaerobacterium saccharolyticum is as shown in SEQ ID. No. 6 below:

(SEQ ID No. 6)
MAHIITDECISCGACAAECPVDAIHEGTGKYEVDADTCIDCGACEPVCP
TGAIKAE

In another embodiment, the microorganism may also contain the pfor gene from Thermoanaerobacterium saccharolyticum. The sequence of the pfor gene from Thermoanaerobacterium saccharolyticum is as shown in SEQ ID. No. 7 below:

(SEQ ID No. 7)
MSKVMKTMDGNTAAAHVAYAFTEVAAIYPITPSSPMAEHVDEWSAHGRK
NLFGQEVKVIEMQSEAGAAGAVHGSLAAGALTTTFTASQGLLLMIPNMY
KIAGELLPGVFHVSARALASHALSIFGDHQDVMACRQTGFALLASGSVQ
EVMDLGSVAHLAAIKGRVPFLHFFDGFRTSHEYQKIEVMDYEDLRKLLD
MDAVREFKKRALNPEHPVTRGTAQNPDIYFQEREASNRYYNAVPEIVEE
YMKEISKITGREYKLFNYYGAPDAERIVIAMGSVTETIEETIDYLLKKG
EKVGVVKVHLYRPFSFKHFMDAIPKTVKKIAVLDRTKEAGAFGEPLYED
VRAAFYDSEMKPIIVGGRYGLGSKDTTPAQIVAVFDNLKSDTPKNNFTI
GIVDDVTYTSLPVGEEIETTAEGTISCKFWGFGSDGTVGANKSAIQIIG
DNTDMYAQAYFSYDSKKSGGVTISHLRFGKKPIRSTYLINNADFVACHK
QAYVYNYDVLAGLKKGGTFLLNCTWKPEELDEKLPASMKRYIAKNNINF
YIINAVDIAKELGLGARINMIMQSAFFKLANIIPIDEAVKHLKDAIVKS
YGHKGEKIVNMNYAAVDRGIDALVKVDVPASWANAEDEAKVERNVPDFI
KNIADVMNRQEGDKLPVSAFVGMEDGTFPMGTAAYEKRGIAVDVPEWQI
DNCIQCNQCAYVCPHAAIRPFLLNEEEVKNAPEGFTSKKAIGKGLEGLN
FRIQVSVLDCTGCGVCANTCPSKEKSLIMKPLETQLDQAKNWEYAMSLS
YKENPLGTDTVKGSQFEKPLLEFSGACAGCGETPYARLVTQLFGDRMLI
ANATGCSSIWGGSAPSTPYTVNKDGHGPAWANSLFEDNAEFGFGMALAV
KQQREKLADIVKEALELDLTQDLKNALKLWLDNFNSSEITKKTANIIVS
LIQDYKTDDSKVKELLNEILDRKEYLVKKSQWIFGGDGWAYDIGFGGLD
HVLASGEDVNVLVFDTEVYSNTGGQSSKATPVGAIAQFAAAGKGIGKKD
LGRIAMSYGYVYVAQIAMGANQAQTIKALKEAESYPGPSLIIAYAPCIN
HGIKLGMGCSQIEEKKAVEAGYWHLYRYNPMLKAEGKNPFILDSKAPTA
SYKEFIMGEVRYSSLAKTFPERAEALFEKAEELAKEKYETYKKLAEQN

### BRIEF DESCRIPTION OF THE DRAWINGS

FIG. 1 shows the metabolic pathway of pyruvate to ethanol in T saccharolyticum. LDH: lactate dehydrogenase; PFL: pyruvate formate-lyase; PFOR: pyruvate ferredoxin oxidoreductase; ALDH, acetaldehyde dehydrogenase; ADH, alcohol dehydrogenase. ALDH and ADH were thought to be catalyzed by bifunctional alcohol dehydrogenase in T. saccharolyticum. Black arrows represent the metabolic pathways; blue arrows represent the cofactor involved in the pathway.

FIG. 2 shows enzymatic activity of pyruvate ferredoxin oxidoreductase from T. saccharolyticum mutants. Error bars represent the standard deviation of three replicates. ND, non-detectable, the specific activities were below detection limit 0.005 U/mg.

FIG. 3 shows growth curves of Δpfor strains in MTC-6 medium with 4.5 g/L yeast extract (A) and without yeast extract (B). Black lines represent wild type strain (LL1025), black circle represent Δpfor-1, green cross represent Δpfor-2, blue diamond represent adapted Δpfor-1, red star represent adapted Δpfor-2.

FIG. 4. Transcriptional data of Tsac_0046 (in pfor_0046 cluster), Tsac_0628 and Tsac_0629 (in pfl_0628 cluster) in pforA deletion strains. recA is (Tsac_1846) is used as a reference gene.

FIG. 5 shows the primary structure of AdhE from wild-type C. thermocellum and T. saccharolyticum. (A) The ALDH domain is shown in light grey (1-423 for C. thermocellum and 1-420 for T. saccharolyticum), ADH domain in white (463-873 for C. thermocellum and 460-860 for T. saccharolyticum) and linker sequence in dark grey (424-462 for C. thermocellum and 421-459 for T. saccharolyticum). The NADH binding sites are shown in grey (“NADH binding site 1” is 200-221 for C. thermocellum and 199-220 for T. saccharolyticum; “NADH binding site 2” is 551-553 for C. thermocellum and 543-545 for T. saccharolyticum) and Fe²⁺ binding site in light grey. Mutated residues discussed in this study are annotated at the appropriate positions. All elements are drawn to scale. (B) and (C) show the sequence conservation of the NADH binding motifs (highlighted in the “Consensus” sequence) of AdhE from Thermoanaerobacter ethanolicus, Thermoanaerobacter mathranii, T. saccharolyticum, Entamoeba histolytica, E. coli, C. thermocellum, Leuconostoc mesenteroids, Lactococcus lactis, Oenococcus oeni and Streptococcus equinus. Residues highlighted in dark grey are of the highest conservation, and white are of the least. The numbering of amino acids is based on the AdhE sequence from C. thermocellum.

FIG. 6 shows PCR confirmation of genetic manipulations of nfnAB in T. saccharolyticum. (A) Schematic for deletion of nfnAB in T. saccharolyticum strains (B) Schematic for insertion of nfnAB under control of the xynA promoter (C) PCR analysis of T. saccharolyticum strains: JW/SL-YS485 (WT), M0353 (Δpta Δack Δldh ΔpyrF, #2), and M1442 (Δpta Δack Δldh adhE^{G544D}, #4), show the expected 3.1 kb external fragment length of nfnAB. Strains LL1144 (ΔnfnAB::Kad^{r}, #1), LL1145 (Δpta Δack Δldh ΔpyrF ΔnfnAB::Kad^{r}, #3), LL1220 (M1442 ΔnfnAB::Kad^{r}, #5) and LL1222 (LL1220 ΔxynA::nfnAB Ery^{r}, #6) with a disrupted nfnAB show a smaller 2.9 kb nfnAB locus fragment. The xynA locus of LL1222 (#6) compared with JW/SL-YS485 (WT) shows the successful integration of nfnAB under control of the xynA promoter, which results in a fragment reduction size from 4.7 kb to 4 kb. The DNA marker is the 1 kb ladder from New England Biolabs.

FIG. 7 shows Native PAGE analysis for NfnAB activity. Cell free extracts were run simultaneously on PAGE gels for 80 minutes, and then incubated in an assay solution consisting of benzyl viologen (BV). NADPH was added to start the reaction. A band of BV reduction corresponding with NfnAB activity was then fixed in the gel with triphenyltetrazolium chloride, marked by the arrow. Lanes loaded with T. saccharolyticum cell free extract (1) JW/SL-YS485 and (2) LL1144 and E. coli cell free extract (3) with pJLO30 expressing nfnAB and (4) with no plasmid. Relevant genotypes are indicated above gels.

FIG. 8 shows Comparison of Thermoanaerobacter and Thermoanaerobacterium genes encoding nfnAB and various alcohol dehydrogenases. Predicted NADPH-linked alcohol/aldehyde dehydrogenases adhA and adhB are found in close proximity to nfnAB, while the predicted NADH-linked adhE bifunctional alcohol/aldehyde dehydrogenases are some distance away. The proteins encoded, when present, share high identity to each other.

FIG. 9 shows proposed electron based models for stoichiometric ethanol production in T. saccharolyticum based on NADPH. NADPH-based ethanol formation relies on the electron transfer from NADH (generated from glyceraldehyde-3-phosphate dehydrogenase) and reduced ferredoxin to 2 NADP⁺, catalyzed by the NfnAB complex. The 2 NADPH are then consumed by NADPH-dependent aldehyde and alcohol dehydrogenase activity.

FIG. 10 shows plasmid maps for expressing the T. saccharolyticum ethanol production pathway (and various subsets thereof). Genes from T saccharolyticum (adhA, nfnA, nfnB and adhE_{G544D}) are indicated with no fill. Plasmid replicons are indicated with black fill. Plasmid backbone genes (replication and antibiotic resistance) are indicated by dark grey fill. Promoters are indicated by light grey fill.

FIG. 11 shows the T. saccharolyticum pathway expressed on a plasmid. This figure shows the relative contribution of each gene to improved ethanol production. Strain LL1004 is wild type C. thermocellum. Strain 477 is the empty vector control. Strains 477 through 484 have plasmids with various combinations of the adhA, nfnA, nfnB and adhE_{G544D} genes from T. saccharolyticum as indicated. The table below the figure shows which genes are present on the plasmid in each strain. For each strain, ethanol production from several colonies was measured. The ethanol production of each colony is indicated by a gray diamond. For each strain, the distribution of ethanol production is shown with a box plot. The box encloses data from the 25^{th} to 75^{th} percentile. The whiskers extend to 1.5 times the inter-quartile range. The dashed line shows the negative control (strain 477, empty plasmid). Cells were grown on 20 g/1 (˜59 mM) cellobiose.

FIG. 12 shows the T. saccharolyticum pathway integrated into the C. thermocellum genome at the C. thermocellum Clo1313_2638 locus, under control of the Clo1313_2638 promoter. C. thermocellum genes are indicated with no fill. The Clo1313_2638 region is indicated with grey fill. The individual T. saccharolyticum genes are indicated with diagonal hatching. The whole T. saccharolyticum pathway (adhA, nfnA, nfnB and adhE_{G544D}) is indicated with black fill.

FIG. 13 shows ethanol production when T. saccharolyticum pathway is inserted onto the C. thermocellum genome. Strains were grown on 20 g/l cellobiose (˜2.9 mmol). Ethanol data is presented in mmol. The fermentation volume was 50 ml, so multiplying by 1000/50 will convert from units of mmol to mM.

### DETAILED DESCRIPTION

Disclosed here are methods to generate microorganisms capable of producing ethanol from lignocellulosic feedstock with high yield. Multiple genes in Thermoanaerobacterium saccharolyticum that are involved in the pyruvate to ethanol pathway are disclosed which may be transferred into C. thermocellum or other natively cellulolytic microorganisms.

Transgenic and exogenous gene expression in C. thermocellum may be performed as described in Olson D G, Giannone R J, Hettich R L, Lynd L R. 2013. Role of the CipA scaffoldin protein in cellulose solubilization, as determined by targeted gene deletion and complementation in Clostridium thermocellum. J Bacteriol 195:733-9. One example of gene expression for metabolic engineering is the expression of the exogenous pyruvate kinase gene from Thermoanaerobacterium saccharolyticum in C. thermocellum. Another example is the complementation of ADH and ALDH activity in C. thermocellum adhE deletion strain. In both of these cases, gene expression was achieved by targeted recombination onto the chromosome, a process which takes several weeks under ideal conditions. See e.g., Olson D G, Lynd L R. 2012. Transformation of Clostridium thermocellum by electroporation. Methods in enzymology, 1st ed. Elsevier Inc.

Plasmid-based gene expression may be performed in single step and may be used in higher throughput metabolic engineering applications. However, there are few reports of successful gene expression in C. thermocellum using replicating plasmids. One attempt to complement the cipA deletion in C. thermocellum saw partial (33% of wild type) restoration of Avicel solubilization. See Olson D G, Giannone R J, Hettich R L, Lynd L R. 2013. Role of the CipA scaffoldin protein in cellulose solubilization, as determined by targeted gene deletion and complementation in Clostridium thermocellum. J Bacteriol 195:733-9. Similarly, a report describing the identifying of native C. thermocellum promoters for use in expressing genes also encountered issues obtaining consistent and reliable results with reporter enzyme activities. See Olson D G, Maloney M, Lanahan A., Hon S, Hauser U, Lynd L R. 2015. Identifying promoters for gene expression in Clostridium thermocellum. Metab Eng Commun 2:23-29.

In one aspect of this disclosure, the term “exogenous” may refer to genes that do not naturally exist in a host organism but are introduced into said host organism. In the present disclosure, certain exogenous genes exist in a different organism, and are introduced into the host organism that does not naturally possess such genes. These exogenous genes may or may not have been modified from their naturally existing forms. In another aspect, the term “exogenous gene” may refer to a foreign gene, namely, a gene (or DNA sequence) that does not exist in the host organism.

The term “biomass” refers to non-fossilized renewable materials that are derived from or produced by living organisms. In its broadest term, biomass may include animal biomass, plant biomass, and human waste and recycled materials, among others. Examples of animal biomass may include animal by-product and animal waste, etc. In one embodiment of this disclosure, biomass refers to plant biomass which includes any plant-derived matter (woody or non-woody) that is available on a sustainable basis. Plant biomass may include, but is not limited to, agricultural crop wastes and residues such as corn stover, corn processing residue such as such as corn bran or corn fiber, wheat straw, rice straw, sugar cane bagasse and the like, grass crops, such as switch grass, alfalfa, winter rye, and the like. Plant biomass may further include, but is not limited to, woody energy crops, wood wastes and residues such as trees, softwood forest thinnings, barky wastes, sawdust, paper and pulp industry residues or waste streams, wood fiber, and the like. In urban areas, plant biomass may include yard waste, such as grass clippings, leaves, tree clippings, brush, etc., vegetable processing waste, as well as recycled cardboard and paper products.

In one embodiment, grassy biomass may be used in the present disclosure. In another embodiment, winter cover crops such as winter rye may be used as a bioenergy feedstock using existing equipment and knowhow. Winter cover crops have little and arguably no competition with food crops for land or revenue, and they also positively impact soil and water quality as well as farm income, and offer important co-product opportunities. A recent study estimated that 200 million dry tons of winter rye per year could be produced in the U.S. on land used to grow corn and soybeans, which has a liquid fuel production potential equal to that of the current U.S. and Brazilian industries combined.

By way of example, a number of embodiments of the present disclosure are listed below:

Item 1. A cellulolytic microorganism comprising an exogenous adhA gene, wherein said adhA gene encodes an alcohol dehydrogenase A having a sequence that is at least 90% identical to the sequence of SEQ ID No. 2.

Item 2. The microorganism of Item 1, wherein said microorganism is a thermophilic bacterium.

Item 3. The microorganism of any one of the preceding items, wherein said microorganism is Clostridium thermocellum.

Item 4. The microorganism of any one of the preceding items, wherein said microorganism is a transgenic microorganism.

Item 5. The microorganism of any one of the preceding items, further comprising an exogenous adhE gene, wherein said adhE gene encodes an aldehyde and alcohol dehydrogenase E having a sequence that is at least 90% identical to the sequence of SEQ ID No. 1.

Item 6. The microorganism of any one of the preceding items, wherein the sequence of the alcohol dehydrogenase A encoded by said adhA gene is at least 99% identical to the sequence of SEQ ID No. 2.

Item 7. The microorganism of any one of the preceding items, wherein the sequence of the aldehyde and alcohol dehydrogenase E encoded by said adhE gene is identical to the sequence of SEQ ID No. 1.

Item 8. The microorganism of any one of the preceding items, wherein the sequence of the alcohol dehydrogenase A encoded by said adhA gene is identical to the sequence of SEQ ID No. 2.

Item 9. The microorganism of any one of the preceding items, further comprising an exogenous nfnA gene, wherein said nfnA gene encodes a protein having a sequence that is at least 90% identical to the sequence of SEQ ID No. 4.

Item 10. The microorganism of any one of the preceding items, further comprising an exogenous nfnB gene wherein said nfnB gene encodes a protein having a sequence that is at least 90% identical to the sequence of SEQ ID No. 5.

Item 11. The microorganism of any one of the preceding items, wherein neither exogenous nfnA nor exogenous nfnB gene is introduced into said microorganism.

Item 12. The microorganism of any one of the preceding items, wherein said aldehyde and alcohol dehydrogenase E has a sequence of SEQ ID No. 3.

Item 13. The microorganism of any one of the preceding items, further comprising an exogenous ferredoxin gene, wherein said exogenous ferredoxin gene encodes a protein having a sequence that is at least 90% identical to the sequence of SEQ ID No. 6.

Item 14. The microorganism of any one of the preceding items, further comprising an exogenous pfor gene, wherein said exogenous pfor gene encodes a protein having a sequence that is at least 90% identical to the sequence of SEQ ID No. 7.

Item 15. A cellulolytic microorganism having a modified pyruvate-to-ethanol pathway, comprising (a) an exogenous adhA gene, (b) an exogenous nfnA gene, (c) an exogenous nfnB gene, (d) an exogenous adhE gene, said exogenous adhA gene encoding an alcohol dehydrogenase A having a sequence that is at least 90% identical to the sequence of SEQ ID No. 2, said exogenous nfnA gene encoding a protein having a sequence that is at least 90% identical to the sequence of SEQ ID No. 4, said exogenous nfnB gene encoding a protein having a sequence that is at least 90% identical to the sequence of SEQ ID No. 5, said exogenous adhE gene encoding a protein having a sequence that is at least 90% identical to the sequence of SEQ ID No. 3.

Item 16. A cellulolytic microorganism having a modified pyruvate-to-ethanol pathway, comprising (a) an exogenous adhA gene, (b) an exogenous nfnA gene, (c) an exogenous nfnB gene, (d) an exogenous ferredoxin gene, (e) an exogenous pfor gene, and (f) an exogenous adhE gene said exogenous adhA gene encoding an alcohol dehydrogenase A having a sequence that is at least 90% identical to the sequence of SEQ ID No. 2, said exogenous nfnA gene encoding a protein having a sequence that is at least 90% identical to the sequence of SEQ ID No. 4, said exogenous nfnB gene encoding a protein having a sequence that is at least 90% identical to the sequence of SEQ ID No. 5, said exogenous ferredoxin gene encoding a protein having a sequence that is at least 90% identical to the sequence of SEQ ID No. 6, said exogenous pfor gene encoding a protein having a sequence that is at least 90% identical to the sequence of SEQ ID No. 7, said exogenous adhE gene encoding a protein having a sequence that is at least 90% identical to the sequence of SEQ ID No. 3.

Item 17. A method of producing ethanol from cellulosic biomass, comprising use of a cellulolytic microorganism having a modified pyruvate-to-ethanol pathway, said cellulolytic microorganism comprising an exogenous adhA gene, wherein said exogenous adhA gene encodes an alcohol dehydrogenase A having a sequence that is at least 90% identical to the sequence of SEQ ID No. 2.

Item 18. The method of Item 17, wherein said microorganism further comprises either or both of nfnA gene and nfnB gene from Thermoanaerobacterium saccharolyticum.

Item 19. The method of any one of the preceding items, wherein said microorganism further comprises the adhE gene from Thermoanaerobacterium saccharolyticum.

Item 20. The method of any one of the preceding items, wherein said microorganism further comprises the pfor gene from Thermoanaerobacterium saccharolyticum.

Item 21. The method of any one of the preceding items, wherein said microorganism further comprises the ferredoxin gene from Thermoanaerobacterium saccharolyticum.

By using the system and methods disclosed herein, other cellulosic feedstocks may also be processed into biofuels without pretreatment. Examples of microorganisms may include but are not limited to C. thermocellum, C. clariflavum, C. bescii, or C. thermocellum/Thermoanaerobacterium saccharolyticum co-culture as fermentation systems. Various techniques known in the art for enhancing ethanol yield may be employed to further enhance the conversion.

It will be readily apparent to those skilled in the art that the systems and methods described herein may be modified and substitutions may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1 Role of Pyruvate Ferredoxin Oxidoreductase and Pyruvate Formate-Lyase in Thermoanaerobacterium saccharolyticum

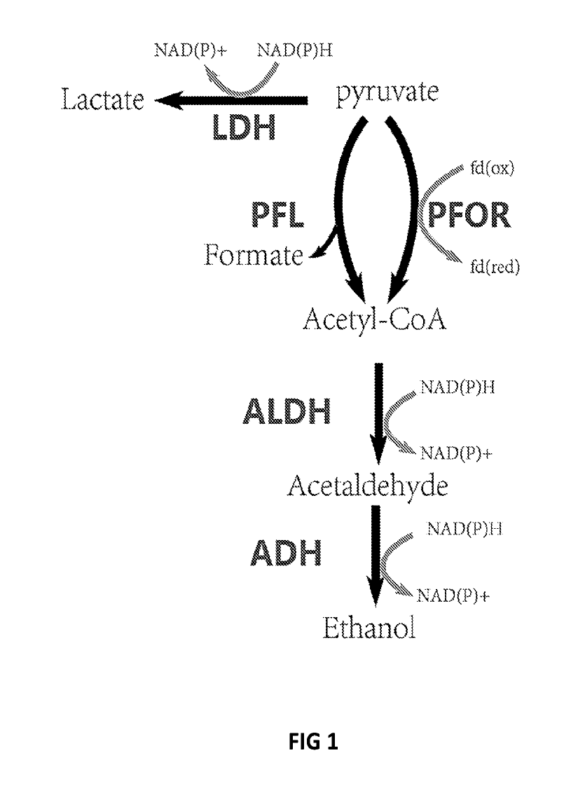

Thermoanaerobacterium saccharolyticum is a thermophilic, anaerobic bacterium able to ferment hemicellulose but not cellulose. Wild-type strains produce ethanol, acetic acid and under some conditions lactic acid as the main fermentation products, but engineered strains produce ethanol at near-theoretical yields and titer of 70 g/l. Hemicellulose-utilizing thermophiles such as T. saccharolyticum commonly accompany cellulolytic microbes in natural environments. The pathway by which engineered strains of T. saccharolyticum produce ethanol may provide examples of high-yield ethanol production involving pyruvate conversion to acetyl-CoA via pyruvate ferredoxin oxidoreductase (PFOR) (FIG. 1), and because of the potential to reproduce this pathway or important features thereof in other thermophiles.

In this Example, genes and enzymes responsible for conversion of pyruvate to acetyl-CoA in Thermoanaerobacterium saccharolyticum were identified using gene deletion. It was found that pyruvate ferredoxin oxidoreductase (PFOR) is encoded by pforA and plays a key role in pyruvate dissimilation. It was further demonstrated that pyruvate formate lyase (PFL) is encoded by pfl. Although the pfl is normally expressed at low levels, it is crucial for biosynthesis in T. saccharolyticum. In pforA deletion strains, pfl expression increased, and was able to partially compensate for the loss of PFOR activity. Deletion of both pforA and pfl resulted in a strain that required acetate for growth and produced lactate as the primary fermentation product, achieving 88% of theoretical lactate yield. Thus, these two enzymes may be the main routes of acetyl-CoA formation from pyruvate in T. saccharolyticum.

The T. saccharolyticum genome includes six genes identified as putative pfor and one gene identified as pfl (Table 1).

TABLE 1
Clusters of pfor and pfl genes

| Gene | | |
|---|---|---|
| cluster | Gene | Annotated gene products^{a} |
| pforA | Tsac_0046 | pyruvate ferredoxin/flavodoxin oxidoreductase |
| pforB | Tsac_0380 | 2-oxoacid: acceptor oxidoreductase subunit |
| | | alpha |
| | Tsac_0381 | pyruvate ferredoxin/flavodoxin oxidoreductase |
| | | subunit beta |
| pforC | Tsac_0915 | pyruvate ferredoxin/flavodoxin oxidoreductase |
| pforD | Tsac_1064 | 4Fe—4S ferredoxin |
| | Tsac_1065 | pyruvate flavodoxin/ferredoxin oxidoreductase |
| | | domain-containing protein |
| | Tsac_1066 | thiamine pyrophosphate TPP-binding domain- |
| | | containing protein |
| | Tsac_1067 | pyruvate/ketoisovalerate oxidoreductase |
| pforE | Tsac_2160 | pyruvate/ketoisovalerate oxidoreductase |
| | Tsac_2161 | thiamine pyrophosphate TPP-binding domain- |
| | | containing protein |
| | Tsac_2162 | pyruvate flavodoxin/ferredoxin oxidoreductase |
| | | domain-containing protein |
| | Tsac_2163 | 4Fe—4S ferredoxin |
| pforF | Tsac_2177 | pyruvate/ketoisovalerate oxidoreductase subunit |
| | | gamma |
| | Tsac_2178 | thiamine pyrophosphate TPP-binding domain- |
| | | containing protein |
| | Tsac_2179 | pyruvate flavodoxin/ferredoxin oxidoreductase |
| | | domain-containing protein |
| | Tsac_2180 | 4Fe—4S ferredoxin |
| pfl | Tsac_0628 | pyruvate formate-lyase |
| | Tsac_0629 | pyruvate formate-lyase activating enzyme |

^{a}The gene product annotations were based on NCBI genome project (NC_017992.1)

Both genomic analysis and enzyme assays suggest that neither pyruvate dehydrogenase nor pyruvate decarboxylase is present in T. saccharolyticum. T saccharolyticum appears to have genes coding all three types of PFOR types defined by Chabriere et al. based on quaternary structure. PFOR enzymes encoded by pforA and pforC are of the homodimer type, the pforB cluster codes for the heterodimer type, and PFORs encoded by cluster pforD, pforE and pforF appears likely to be the heterotetramer type. The PFOR reaction is shown by Table 2, reaction [A]. There are conflicting data presented in the literature about which genes are responsible for encoding PFOR. Shaw et al. identified Tsac_0380 and Tsac_0381 as the main pfor genes and detected methyl viologen dependent PFOR activity in wild type T. saccharolyticum. However, proteomic analysis indicates that PFOR encoded by pforA is the most abundant PFOR in glucose grown cells (12). The PFL reaction is shown by Table 2, equation [C]. Shaw et al. identified Tsac_0628 as the gene encoding PFL. However, formate has not been detected as a fermentation product in either the wild type nor the high-ethanol-producing strain ALK2.

TABLE 2
Potential reactions related to pyruvate dissimilation in T.
saccharolyticum

| | Enzyme name | Reaction catalyzed by the enzyme |
|---|---|---|
| A | Pyruvate ferredoxin | pyruvate + CoA + ferredoxin (ox) → |
| | oxidoreductase (PFOR) | acetyl-CoA + ferredoxin (red) + |
| | | CO₂ |
| B | Ferredoxin/NAD(P)H | 2 ferredoxin (red) + NAD(P)⁺ + H⁺→2 |
| | oxidoreductase (FNOR) | ferredoxin (ox) + NAD(P)H |
| C | Pyruvate formate lyase | pyruvate + CoA → acetyl-CoA + |
| | (PFL) | formate |
| D | Formate dehydrogenase | formate + NAD(P)⁺ → CO₂ + |
| | (FDH) | NAD(P)H |

In fermentative microbes with catabolism featuring pyruvate conversion to acetyl-CoA, the electrons from this oxidation must end up in ethanol, presumably via nicotinamide cofactors, in order for the ethanol yield to exceed one mole per mole hexose. In the case of PFOR, this means that electrons from reduced ferredoxin need to be transferred to NAD⁺ or NADP⁺. In the case of PFL, this means that electrons from formate must be transferred to NAD⁺ or NADP⁺. Shaw et al. have detected ferredoxin-NAD(P)H activity, corresponding to reaction [B] in Table 2, in cell extracts. A fnor gene (Tsac_2085) has also been identified. However formate dehydrogenase, equation [D] in Table 2, has not been found in T. saccharolyticum either by genome homology or enzyme assay.

The conversion of pyruvate to acetyl-CoA is always thought to be carried out by PFOR in T. saccharolyticum. However, few of the specific genes and enzymes responsible for ethanol formation from pyruvate in T. saccharolyticum have been unambiguously identified. One of the objectives of the experiments in this Example is to confirm if PFOR is responsible for pyruvate dissimilation, and to identify which of the many PFOR enzymes is most important. Another objective is to gain insight into the function of PFL, and to examine the physiological consequences of deleting these genes individually and in combination.

Strains and Plasmids.

Strains and plasmids described in this document are listed in Table 3.

TABLE 3
Strains and plasmids

| | | Accession | |
|---|---|---|---|
| Strain or Plasmid | Description^{a} | number | Reference |
| Strains | | | |
| E. coli | | | |
| DH5α | E. coli cloning strains | N/A | New |
| | | | England |
| | | | Biolabs |
| T. | | | |
| saccharolyticum | | | |
| LL1025 | wild type strain | SRA234880 | [31] |
| LL1040 (aka | high-ethanol-producing | N/A | [2] |
| ALK2) | strain, Kan^{r}, Erm^{r} | | |
| LL1049 | high-ethanol-producing | SRA233073 | [4] |
| | strain | | |
| LL1139 | LL1025 ΔpforA ::Kan^{r}, | SRA234882 | This study |
| | colony 1 | | |
| LL1140 | LL1025 ΔApforA ::Kan^{r}, | SRA233066 | This study |
| | colony 2 | | |
| LL1141 | Adapted LL1139 | SRA234883 | This study |
| LL1142 | Adapted LL1140 | SRA234884 | This study |
| LL1155 | LL1025 ΔpforD :: Kan^{r}, | N/A | This study |
| LL1156 | LL1025 ΔpforB :: Kan^{r}, | N/A | This study |
| LL1157 | LL1025 ΔpforF :: Kan^{r}, | N/A | This study |
| LL1159 | LL1049 ΔpforA :: Kan^{r}, | N/A | This study |
| LL1164 | LL1025 Δpfl :: Kan^{r}, | SRA233080 | This study |
| | colony 1 | | |
| LL1170 | LL1025 Δpfl :: Kan^{r}, | SRA233074 | This study |
| | colony 2 | | |
| LL1178 | LL1141 Δpfl :: Erm^{r} | SRA234885 | This study |
| Plasmids | | | |
| pMU433 | Cloning vector pta/ack, Kan^{r} | | [32] |
| pZJ13 | pforA knock out, Kan^{r}, pta/ | KP057684 | This study |
| | ack | | |
| pZJ15 | pforB knock out, Kan^{r}, pta/ | KP057685 | This study |
| | ack | | |
| pZJ16 | pforD knock out, Kan^{r}, pta/ | KP057686 | This study |
| | ack | | |
| pZJ17 | pforF knock out, Kan^{r}, pta/ | KP057687 | This study |
| | ack | | |
| pZJ20 | pfl knock out, Kan^{r}, pta/ack | KP057688 | This study |
| pZJ23 | Cloning vector Erm^{r}, Amp^{r} | KP057689 | This study |
| pZJ25 | pfl knock out, Erm^{r}, Amp^{r} | KP057690 | This study |

^{a}Kan^{r}, kanamycin resistant;
Erm^{r}, erythromycin resistant;
Amp^{r}, ampicillin resistant;
pta/ack is a negative selective marker.

Media and Growth Conditions.

Genetic modifications of T saccharolyticum JW/SL-YS485 strains were performed in CTFUD medium, containing 1.3 g/L (NH₄)₂SO₄, 1.5 g/L KH₂PO₄, 0.13 g/L CaCl₂.2H₂O, 2.6 g/L MgCl₂.6H₂O, 0.001 g/L FeSO₄.7H₂O, 4.5 g/L yeast extract, 5 g/L cellobiose, 3 g/L sodium citrate tribasic dihydrate, 0.5 g/L L-cysteine-HCl monohydrate, 0.002 g/L resazurin and 10 g/L agarose (for solid media only). The pH was adjusted to 6.7 for selection with kanamycin (200 μg/ml), or pH was adjusted to 6.1 for selection with erythromycin (25 μg/ml).

Measurement of fermentation products and growth of T. saccharolyticum were performed in MTC-6 medium, including 5 g/L cellobiose, 9.25 g/L MOPS (morpholinepropanesulfonic acid) sodium salt, 2 g/L ammonium chloride, 2 g/L potassium citrate monohydrate, 1.25 g/L citric acid monohydrate, 1 g/L Na₂SO₄, 1 g/L KH₂PO₄, 2.5 g/L NaHCO₃, 2 g/L urea, 1 g/L MgCl₂.6H₂O, 0.2 g/L CaCl₂.2H₂O, 0.1 g/L FeCl₂.6H₂O, 1 g/L L-cysteine HCl monohydrate, 0.02 g/L pyridoxamine HCl, 0.004 g/L p-aminobenzoic acid (PABA), 0.004 g/L D-biotin, 0.002 g/L Vitamin B12, 0.04 g/L thiamine, 0.005 g/L MnCl₂.4H₂O, 0.005 g/L CoCl₂.6H₂O, 0.002 g/L ZnCl₂, 0.001 g/L CuCl₂.2H₂O, 0.001 g/L H₃BO₃, 0.001 g/L Na₂MoO₄.2H₂O, 0.001 g/L NiCl₂.6H₂O. It was prepared by combining six sterile solutions with minor modification under nitrogen atmosphere as described before. All of six solutions were sterilized through a 0.22 μm filter (Corning, #430517). A solution, concentrated 2.5-fold, contained cellobiose, MOPS sodium salt and distilled water. B Solution, concentrated 25-fold, contained potassium citrate monohydrate, citric acid monohydrate, Na₂SO₄, KH₂PO₄, NaHCO₃ and distilled water. C solution, concentrated 50-fold, contained ammonium chloride and distilled water. D solution, concentrated 50-fold, contained MgCl₂.6H₂O, CaCl₂.H₂O, FeCl₂.6H₂O, L-cysteine HCl monohydrate. E solution, concentrated 50-fold, contained thiamine, pyridoxamine HCl, p-aminobenzoic acid (PABA), D-biotin, Vitamin B12. F solution, concentrated 1000-fold, contained MnCl₂.4H₂O, CoCl₂.6H₂O, ZnCl₂, CuCl₂.2H₂O, H₃BO₃, Na₂MoO₄.2H₂O, NiCl₂.6H₂O. For some fermentation required additional compositions, additional compositions were added after six solutions were combined. The pH was adjusted to 6.1 as the optimal pH for growth. Fermentations of T. saccharolyticum were done in 125-ml glass bottles at 55° C. under nitrogen atmosphere. The working volume is 50 ml with shaking at 250 rpm. Fermentations were allowed to proceed for 72 h at which point samples were collected for analysis.

Growth curve and maximum OD measurement were determined in a 96-well plate incubated in the absence of oxygen as previously described. Each well contained 200μl MTC-6 medium. And the plate was shaken for 30 seconds every 3 minutes, followed by measuring the optical density at 600 nm.

E. coli strains used for cloning were grown aerobically at 37° C. in Lysogeny Broth (LB) medium with either kanamycin (200 μg/ml) or erythromycin (25 μg/ml). For cultivation on solid medium, 15 g/L agarose was added.

All reagents used were from Sigma-Aldrich unless otherwise noted. All solutions were made with water purified using a MilliQ system (Millipore, Billerica, Mass.).

Plasmid Construction.

Plasmids for gene deletion were designed as previously described with either kanamycin or erythromycin resistance cassettes from plasmids pMU433 or pZJ23 flanked by 1.0 to 0.5-kb regions homologous to the 5′ and 3′ regions of the deletion target of interest. Plasmids pZJ13, pZJ15, pZJ16, pZJ17 and pZJ20 were created based on pMU433. The backbone and kanamycin cassette from plasmid pMU433 were amplified by the primers shown in Table 4. Homologous regions of deletion targets of interest were amplified from wild type T. saccharolyticum (LL1025). Plasmid pZJ23 was created as a new deletion vector by assembling an erythromycin cassette from the ALK2 strain and E. coli replication region from plasmid pUC19. Plasmid pZJ25 was based on pZJ23 with homologous regions inserted to allow deletion of pfor_0628. The same homologous region on pZJ20 were amplified and cloned on pZJ25.

TABLE 4
Oligonucleotides described in this document

| Primer | Target gene | Sequence (5′-3′) (SEQ ID No.) |
|---|---|---|
| JP75 | Kanamycin cassette from pMU433 | TAAACCGCTAAGGCATGA (8) |
| JP76 | | CTATCTGCATCGTCTTTTC (9) |
| JP77 | pMU433 backbone | AGTTAGGATGTTGGCAGA (10) |
| JP78 | | AAAGAGGGCATACAAGGA (11) |
| JP209 | Erythromycin cassette from ALK2 | TGCAGGTCGATAAACCCAG (12) |
| JP210 | | GAATTCCCTTTAGTAACGTGTAACTTTC (13) |
| JP211 | Replication region from pUC19 | CATTAATGAATCGGCCAAC (14) |
| JP212 | | CTCGTGATACGCCTATTT (15) |
| JP143 | external to pforA cluster | GCTGTGGCAACTTAACAA (16) |
| JP144 | | CTCATATCATCCGCTCCT (17) |
| JP167 | external to pforB cluster | GTTGTTGTTTTGGCTTAGG (18) |
| JP168 | | AGGCTTTCATTCAGTACG (19) |
| JP169 | external to pforD cluster | CGTGCCTTTTGACCTTCC (20) |
| JP170 | | CTGCTGTCTCGTCCTATT (21) |
| JP171 | external to pforF cluster | CCAATATACCACCAGCCA (22) |
| JP172 | | GAATTTAGGAAAACCGCCA (23) |
| JP181 | external to pfl cluster | ATCCCTCTGTGTCTTTATC (24) |
| JP182 | | TGGTTGTGGGTGTTTATG (25) |
| recA-F | qPCR for Tsac_1846 (recA) | GAAGCCTTAGTGCGAAGTGG (26) |
| recA-R | | GAAGTCCAACATGTGCATCG (27) |
| pfor-F | qPCR for Tsac_0046 | ATCAAGCTTGGAATGGGTTG (28) |
| pfor-R | | GCTGTTGGAGCCTTTGAGTC (29) |
| pfl-F | qPCR for Tsac_0628 | CTATAGCATCGCCTGCTGTG (30) |
| pfl-R | | TCGATACCGCCGTTTATAGC (31) |
| pfl_ae-F | qPCR for Tsac_0629 | ATTGCCATAACCCTGACACA (32) |
| pfl_ae-R | | TAGGCTCTCCACCTGTCAGC (33) |

Plasmids were assembled by Gibson assembly master mix (New England Biolabs, Ipswich, Mass.). The assembled circular plasmids were transformed into E. coli DH5a chemical competent cells (New England Biolabs, Ipswich, Mass.) for propagation. Plasmids were purified by a Qiagen miniprep kit (Qiagen Inc., Germantown, Md.).

Transformation of T. saccharolyticum.

Plasmids were transformed into naturally-competent T. saccharolyticum as described before. Mutant were grown and selected on solid medium with kanamycin (200 μg/ml) at 55° C. or with erythromycin (20 μg/ml) at 48° C. in an anaerobic chamber (COY Labs, Grass Lake, Mich.). Mutant colonies appeared on selection plates after about 3 days. Target gene deletions with chromosomal integration of both homology regions were confirmed by PCR with primers external to the target genes (Table 4).

Preparation of Cell-Free Extracts.

T. saccharolyticum cells were grown in CTFUD medium in an anaerobic chamber (COY labs, Grass Lake, Mich.), and harvested in the exponential phase of growth. To prepare cell-free extracts, cells were collected by centrifugation at 6000 g for 15 minutes and washed twice under similar conditions with a deoxygenated buffer containing 100 mM Tris-HCl (pH 7.5 at 0° C.) and 5 mM dithiothreitol (DTT). Cells were resuspended in 3 ml of the washing buffer. Resuspended cells were lysed by adding 10⁴ U of Ready-lyse lysozyme solution (Epicentre, Madison, Wis.) and 50 U of DNase (Thermo scientific, Waltham, Mass.) and then incubated at room temperature for 20 minutes. The crude lysate was centrifuged at 12,000 g for 5 minutes and the supernatant was collected as cell-free extract. The total amount of protein in the extract was determined by Bradford assay, using bovine serum albumin as the standard.

Enzymes Assays.

Enzyme activity was assayed in an anaerobic chamber (COY labs, Grass Lake, Mich.) using an Agilent 8453 spectrophotometer with Peltier temperature control module (part number 89090A) to maintain assay temperature. The reaction volume was 1 ml, in reduced-volume quartz cuvettes (part number 29MES10; Precision Cells Inc., NY) with a 1.0 cm path length. All enzyme activities are expressed as μmol of product·min⁻¹·(mg of cell extract protein)⁻¹. For each enzyme assay, at least two concentrations of cell extract were used to confirm that specific activity was proportional to the amount of extract added.

All chemicals and coupling enzymes were purchased from Sigma except for coenzyme A, which is from EMD Millipore (Billerica, Mass.). All chemicals were prepared fresh weekly.

Pyruvate ferredoxin oxidoreductase was assayed by the reduction of methyl viologen at 578 nm at 55° C. with minor modifications as described before. An extinction coefficient of X578=9.7 mM⁻¹ cm⁻¹ was used for calculating activity. The assay mixture contained 100 mM Tris-HCl (pH=7.5 at 55° C.), 5 mM DTT, 2 mM MgCl₂, 0.4 mM coenzyme A, 0.4 mM thiamine pyrophosphate, 1 mM methyl viologen, cell extract and approximately 0.25 mM sodium dithionite (added until faint blue, A₅₇₈=0.05-0.15). The reaction was started by adding 10 mM sodium pyruvate.

Adaptation Experiment.

Inside the anaerobic chamber, strains were inoculated into polystyrene tubes (Corning, Tewksbury, Mass.), containing 10 ml MTC-6 medium. The growth of cells in culture was determined by measuring OD₆₀₀. 200 μl of cultures was transferred into tubes with 10 ml fresh medium at the exponential phase of growth as indicated by OD_{600 nm}=0.3.

RNA isolation, RT-PCR and qPCR for determining transcriptional expression level.

3 ml of bacterial culture was pelleted and lysed by digestion with lysozyme (15 mg/ml) and proteinase K (20 mg/ml). RNA was isolated with an RNeasy minikit (Qiagen Inc., Germantown, Md.) and digested with TURBO DNase (Life Technologies, Grand Island, N.Y.) to remove contaminating DNA. cDNA was synthesized from 500 ng of RNA using the iScript cDNA synthesis kit (Bio-Rad, Hercules, Calif.). Quantitative PCR (qPCR) was performed using cDNA with SsoFast EvaGreen Supermix (Bio-Rad, Hercules, Calif.) at an annealing temperature of 55° C. to determine expression levels of Tsac_0046, Tsac_0628 and Tsac_0629. In each case, expression was normalized to recA RNA levels. In order to confirm removal of contaminating DNA from RNA samples, cDNA was synthesized in the presence and absence of reverse transcriptase followed by qPCR using recA primers to insure only background levels were detected in the samples lacking reverse transcriptase. Standard curves were generated using a synthetic DNA template (gBlock, IDT, Coralville, Iowa) containing the amplicons. Primers used for qPCR are listed in Table 4.

Genomic Sequencing.

Genomic DNA was submitted to the Joint Genome Institute (JGI) for sequencing with an Illumina MiSeq instrument. Paired-end reads were generated, with an average read length of 150 bp and paired distance of 500 bp. Raw data was analyzed using CLC Genomics Workbench, version 7.5 (Qiagen, USA). First reads were mapped to the reference genome (NC_017992). Mapping was improved by 2 rounds of local realignment. The CLC Probabilistic Variant Detection algorithm was used to determine small mutations (single and multiple nucleotide polymorphisms, short insertions and short deletions). Variants occurring in less than 90% of the reads and variants that were identical to those of the wild type strain (i.e. due to errors in the reference sequence) were filtered out. The fraction of the reads containing the mutation is presented in Table S1.

To determine larger mutations, the CLC InDel and Structural Variant algorithm was run. This tool analyzes unaligned ends of reads and annotates regions where a structural variation may have occurred, which are called breakpoints. Since the read length averaged 150 bp and the minimum mapping fraction was 0.5, a breakpoint can have up to 75 bp of sequence data. The resulting breakpoints were filtered to eliminate those with fewer than 10 reads or less than 20% “not perfectly matched.” The breakpoint sequence was searched with the Basic Local Alignment Search Tool (BLAST) algorithm for similarity to known sequences. Pairs of matching left and right breakpoints were considered evidence for structural variations such as transposon insertions and gene deletions. The fraction of the reads supporting the mutation (left and right breakpoints averaged) is presented in Table 51.

Unamplified libraries were generated using a modified version of Illumina's standard protocol. 100 ng of DNA was sheared to 500 bp using a focused-ultrasonicator (Covaris). The sheared DNA fragments were size selected using SPRI beads (Beckman Coulter). The selected fragments were then end-repaired, A-tailed, and ligated to Illumina compatible adapters (IDT, Inc) using KAPA-Illumina library creation kit (KAPA biosystems). Libraries were quantified using KAPA Biosystem's next-generation sequencing library qPCR kit and run on a Roche LightCycler 480 real-time PCR instrument. The quantified libraries were then multiplexed into pools for sequencing. The pools were loaded and sequenced on the Illumina MiSeq sequencing platform utilizing a MiSeq Reagent Kit v2 (300 cycle) following a 2×150 indexed run recipe.

Analytical Techniques.

Fermentation products: cellobiose, glucose, acetate, lactate, formate, pyruvate, succinate, malate and ethanol were analyzed by a Waters (Milford, Mass.) high pressure liquid chromatography (HPLC) system with an Aminex HPX-87H column (Bio-Rad, Hercules, Calif.). The column was eluted at 60° C. with 0.25 g/L H₂SO₄ at a flow rate of 0.6 ml/min Cellobiose, glucose, acetate, lactate, formate, succinate, malate and ethanol were detected by a Waters 410 refractive-index detector and pyruvate was detected by a Waters 2487 UV detector. Sample collection and processing were as reported previously.

Carbon from cell pellets were determined by elemental analysis with a TOC-V CPH and TNM-I analyzer (Shimadzu, Kyoto, Japan) operated by TOC-Control V software. Fermentation samples were prepared as described with small modifications. A 1 ml sample was centrifuged to remove supernatant at 21,130×g for 5 minutes at room temperature. The cell pellet was washed twice with MilliQ water. After washing, the pellet was resuspended in a TOCN 25 ml glass vial containing 19.5 ml MilliQ water. The vials were then analyzed by the TOC-V CPH and TNM-I analyzer.

Hydrogen was determined by gas chromatography using a Model 310 SRI Instruments (Torrence, Calif.) gas chromatograph with a HayeSep D packed column using a thermal conductivity detector and nitrogen carrier gas. The nitrogen flow rate was 8.2 ml/min.

Carbon balances were determined according to the following equations, with accounting of carbon dioxide and formate through the stoichiometric relationship of its production to levels of acetate, ethanol, malate and succinate. The overall carbon balance is as follows:

Cₜ=12CB+6G+3L+3A+3E+3P+3M+3S+1Pe

Where Cₜ=total carbon, CB=cellobiose, G=glucose, L=lactate, E=ethanol, P=pyruvate, M=malate, S=succinate, Pe=pellet and

$${C}_{R} = \frac{{C}_{tf}}{{C}_{t 0}} \times 100 \%$$

Where C_{R}=carbon recovery, Cₜ₀=total carbon at the initial time, and C_{tf}=total carbon at the final time. Electron recoveries were calculated in a similar way, with following numbers of available electrons per mole of compound: per mole 48 for cellobiose, 24 for glucose, 8 for acetate, 12 for ethanol, 12 for lactate, 14 for succinate, 10 for pyruvate, 12 for malate, 2 for hydrogen and 2 for formate. The electrons contained in the cell pellet was estimated with a general empirical formula for cell composition (CH₂N_{0.25}O_{0.5}), therefore, the available electrons per mole cell carbon was assumed to be 4.75 per mole. The calculation follows the equations below:

$${E}_{t} = 48 CB + 24 G + 12 L + 8 A + 12 E + 14 S + 10 P + 12 M + 2 H + 2 F + 4.75 Pe$$

$${E}_{R} = \frac{{E}_{tf}}{{E}_{t 0}} \times 100 \%$$

Where Eₜ=total electrons, E_{R}=electron recovery, F=formate, H=hydrogen, other abbreviations are the same shown above.

Deletion of pfor.

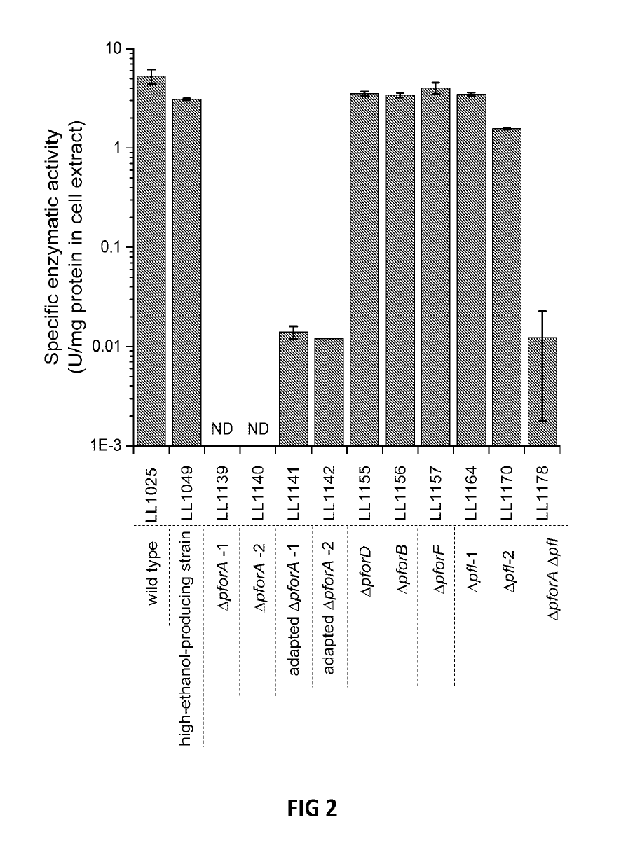

There are six gene clusters in the T. saccharolyticum genome annotated as pyruvate ferredwdn/flavodwdn oxidoreductases according to KEGG (Table 1). In the first round of deletions, 4 of the 6 clusters were deleted: pforA, pforB, pforD and pforF separately in the wild type strain (LL1025). Deletion of pforA resulted in the elimination of PFOR enzyme activity. The other deletions did not affect PFOR activity (FIG. 2). As expected from the enzyme assay data, only the pforA deletion resulted in a change in fermentation products (Table 5).

TABLE 5
Fermentation profiles of T. saccharolyticum knockout strains^{a}

| | | | Fermentation profile^{b} | | | | | | |
|---|---|---|---|---|---|---|---|---|---|
| | | | Unit: mmol in 50 ml culture | | | | | | |
| Strains | | Additions to | Consumed | Residual | | | | | |
| Name | Description | medium | cellobiose | cellobiose | Formate | Lactate | Acetate | Ethanol | Pyruvate |
| LL1025 | Wild type | None | 0.70 | 0.00 | 0.01 | 0.28 | 0.71 | 1.18 | 0.01 |
| LL1049 | Ethanologenic strain | None | 0.70 | 0.00 | 0.22 | 0.00 | 0.04 | 2.10 | 0.00 |
| LL1139 | LL1025 ΔpforA-1 | None | 0.07 | 0.63 | 0.15 | 0.04 | 0.05 | 0.09 | 0.00 |
| LL1140 | LL1025 ΔpforA-2 | None | 0.02 | 0.68 | 0.01 | 0.00 | 0.00 | 0.00 | 0.00 |
| LL1141 | Adapted from LL1139 | None | 0.37 | 0.34 | 0.28 | 0.67 | 0.06 | 0.29 | 0.00 |
| LL1142 | Adapted from LL1140 | None | 0.34 | 0.36 | 0.38 | 0.26 | 0.03 | 0.42 | 0.02 |
| LL1155 | LL1025 ΔpforD | None | 0.70 | 0.00 | 0.02 | 0.39 | 0.72 | 1.20 | 0.01 |
| LL1156 | LL1025 ΔpforB | None | 0.70 | 0.00 | 0.02 | 0.15 | 0.80 | 1.24 | 0.01 |
| LL1157 | LL1025 Δpfor_2177 | None | 0.70 | 0.00 | 0.00 | 0.38 | 0.76 | 1.19 | 0.00 |
| LL1159 | LL1049 ΔpforA | None | 0.07 | 0.63 | 0.01 | 0.00 | 0.00 | 0.01 | 0.00 |
| LL1164 | LL1025 Δpfl-1 | Formate | 0.48 | 0.21 | −0.02^{c} | 1.13 | 0.19 | 0.33 | 0.00 |
| LL1170 | LL1025 Δpfl-2 | Formate | 0.69 | 0.00 | −0.03^{c} | 0.24 | 0.81 | 1.22 | 0.00 |
| LL1178 | LL1025 ΔpforA; Δpfl | Formate and | 0.48 | 0.25 | −0.01^{d} | 1.69 | −0.12^{d} | 0.08 | 0.00 |
| | | Acetate | | | | | | | |

| | | Fermentation profile^{b} | | | | | |
|---|---|---|---|---|---|---|---|
| | | Unit: mmol in 50 ml culture | | | | | |
| Strains | | | | Pellet | | Carbon | Electron |
| Name | Description | Succinate | Malate | carbon | Hydrogen | recovery | recovery |
| LL1025 | Wild type | 0.00 | 0.00 | 0.80 | 1.68 | 90% | 94% |
| LL1049 | Ethanologenic strain | 0.00 | 0.04 | 0.72 | 0.22 | 86% | 90% |
| LL1139 | LL1025 ΔpforA-1 | 0.00 | 0.00 | 0.18 | 0.01 | 98% | 99% |
| LL1140 | LL1025 ΔpforA-2 | 0.00 | 0.00 | 0.19 | 0.10 | 100% | 101% |
| LL1141 | Adapted from LL1139 | 0.06 | 0.03 | 0.27 | 0.02 | 91% | 92% |
| LL1142 | Adapted from LL1140 | 0.22 | 0.03 | 0.21 | 0.04 | 89% | 90% |
| LL1155 | LL1025 ΔpforD | 0.00 | 0.00 | 0.72 | 1.65 | 91% | 94% |
| LL1156 | LL1025 ΔpforB | 0.00 | 0.00 | 0.89 | 1.74 | 92% | 94% |
| LL1157 | LL1025 Δpfor_2177 | 0.00 | 0.00 | 0.73 | 1.64 | 91% | 94% |
| LL1159 | LL1049 ΔpforA | 0.00 | 0.00 | 0.23 | 0.05 | 92% | 92% |
| LL1164 | LL1025 Δpfl-1 | 0.00 | 0.00 | 0.47 | 0.48 | 96% | 98% |
| LL1170 | LL1025 Δpfl-2 | 0.00 | 0.00 | 0.92 | 0.69 | 92% | 95% |
| LL1178 | LL1025 ΔpforA; Δpfl | 0.00 | 0.01 | 0.31 | 0.00 | 94% | 96% |

^{a}Amount of fermentation end-products are reported in millimoles in a volume of 50 ml serum bottle. The amounts of Initial cellobiose were 0.70 mmol for all fermentation. Cultures were incubated for 72 h at 55° C. with an initial pH of 6.2 in MTC-6 medium.
^{b}The standard deviations were less than 10% for cellobiose, formate, lactate, acetate, ethanol, pyruvate, succinate, malate, which were measured by HPLC. For pellet carbon and hydrogen measurement, the standard deviation was less than 2%. The calculated carbon recovery and electron recovery has a combined standard deviation less than 5%.
^{c}In order to improve the growth of LL1164, LL1170, 0.20 millimoles formate were added into 50 ml MTC-6 medium. Negative values represent certain amount of sodium formate was consumed during fermentation.
^{d}LL1178 requires supplementation of both formate and acetate to grow in MTC-6 medium. 0.20 millimoles sodium formate and 0.20 millimoles sodium acetate were added into 50 ml MTC-6 medium. Negative values represent certain amount of sodium formate and sodium acetate was consumed during fermentation

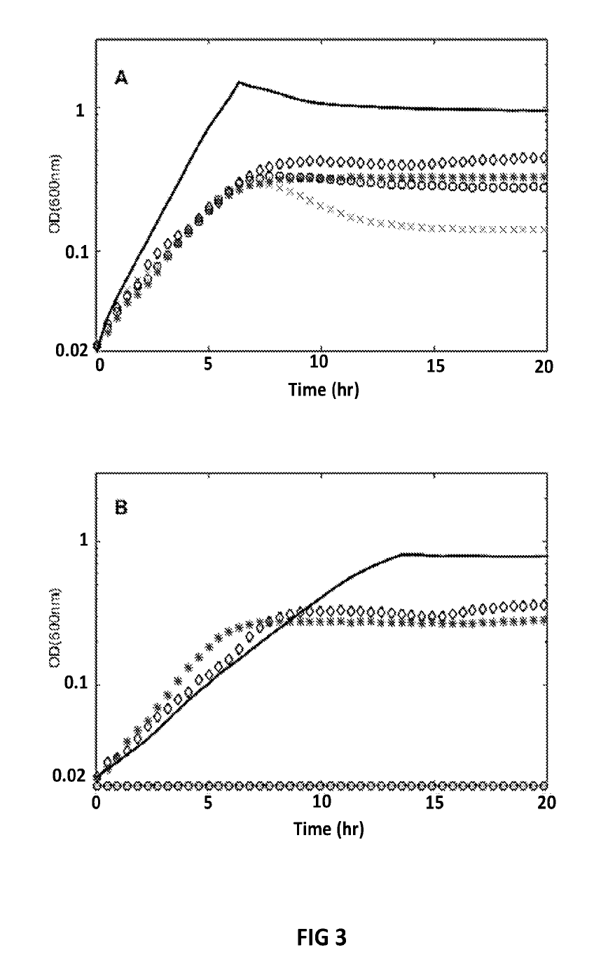

Fermentation profiles for individual colonies of the pforA deletion strain revealed two different phenotypes, which were stored as strains LL1139 and LL1140, respectively. Both LL1139 and LL1140 showed elevated formate production compared to the wild type strain. LL1140 had less lactate production than LL1139 (Table 5). Both of them had defective growth (FIG. 3A) and were not able to consume more than 10% of the 5 g/l cellobiose initially present in the medium (Table 5). Of eight colonies analyzed, seven had the LL1139 phenotype and only one had the LL1140 phenotype.

In order to improve strain fitness, both LL1139 and LL1140 were adapted in MTC-6 medium for 20 transfers (approximately 140 generations) until no additional changes in growth rate were observed. Adapted version of strains LL1139 and LL1140 were named LL1141 and LL1142 respectively. Both strains produced more formate compared with their un-adapted parent strains. LL1141 produced more lactate and less pyruvate than LL1142, but otherwise their fermentation profiles were similar Both strains were able to consume about half of the 5 g/l cellobiose initially present in the medium (Table 5) and the maximum cell density and growth rate were greater than the un-adapted parent strains in defined medium but did not recover to wild type level (FIG. 3B).

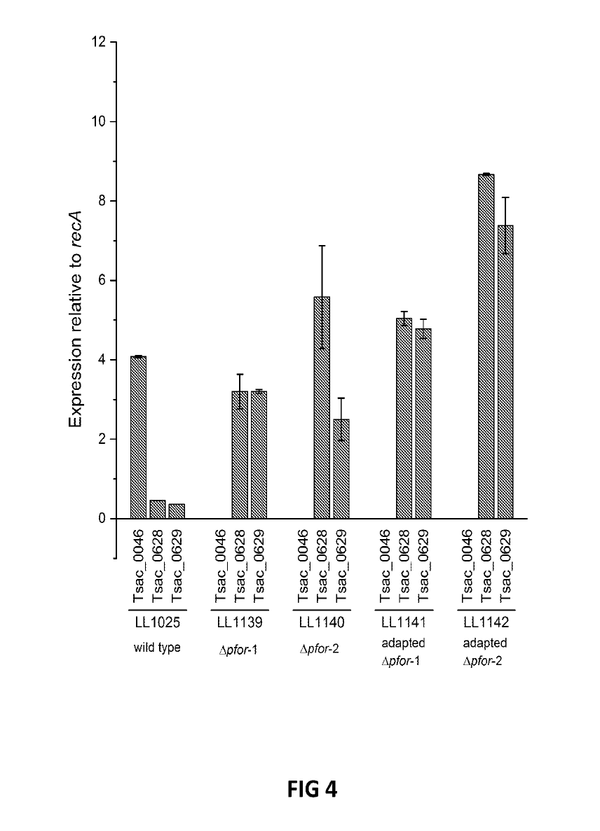

In all pfor deletion strains, the expression levels of pyruvate formate lyase genes were increased at least 6-fold compared with the parent strain (FIG. 4). Consistent with the higher formate production in LL1142, the transcriptional analysis also indicated pfl had higher expression level in LL1142 than LL1141.

pforA was also deleted in the high-ethanol-producing strain of T saccharolyticum, LL1049, previously developed by Mascoma. The resulting strain was named LL1159. This strain grew slower than LL1139 or LL1140 in MTC-6 medium and it was unable to consume more than 10% of 5 g/L cellobiose (Table 5).

Deletion of pfl.

In order to investigate the physiological role of PFL in T. saccharolyticum, the pfl gene cluster was deleted in the wild type (LL1025). The pfl deletion in strain LL1025 gave two different phenotypes, which were stored as strain LL1164 and LL1170. Of eight colonies picked, two had the LL1164 phenotype and six had the LL1170 phenotype. Strain LL1170 consumed more cellobiose, produced more acetate and ethanol and less lactate than strain LL1164 (Table 5).

Both pfl deletion strains grew more poorly MTC-6 medium than in CTFUD medium. The biggest difference between CTFUD and MTC-6 medium is the presence of yeast extract. Additional yeast extract could restore the growth of pfl deletions strains in MTC-6 medium. The growth of both strains was stimulated by addition of formate, serine or lipoic acid. In the presence of added formate, all three strains consumed a small amount (less than 1 mM, which is equivalent to 0.05 millimoles in 50 ml culture as shown in Table 5).

Double Deletion of pfor and pfl.

In the adapted pforA deletion strains (LL1141 and LL1142), formate production was significantly increased, and carbon flux towards acetate and ethanol formation was presumptively via PFL. To show that PFOR encoded by pforA and PFL encoded by pfl were the only two routes for the conversion of pyruvate to ethanol in T. saccharolyticum, pfl was deleted in strain LL1141 (which already contained the pforA deletion). In order to create this deletion, the medium was supplemented with 4 mM sodium acetate.

The resulting pfor/pfl double deletion strain (LL1178) consumed about 70% of the 5 g/l cellobiose initially present, which was about the same as its parent strain (LL1141). It required sodium acetate for growth, even in the presence of yeast extract. Lactate became the main fermentation product, with 3.5 moles of lactate produced for each mole of cellobiose consumed (or 88% of the theoretical maximum yield) (Table 5).

Genomic Sequence of Mutants.

By comparing the resequencing results for the pfor deletion strains (LL1139 and LL1140) (Table 3), a mutation was found in lactate dehydrogenase of LL1140, which was maintained during the adaptation process and also found in strain LL1142 (adapted version of LL1140).

As described before, two different phenotypes were isolated when pfl was deleted in T. saccharolyticum. They were named as LL1164 and LL1170, respectively. LL1164 cannot consume 5 g/L cellobiose and produce lactate as main fermentative product. After comparing the genomic resequencing data of LL1164 and LL1170, two phenotypes of pfl deletion strains from wild type T. saccharolyticum, two mutations were found in LL1164 but not in LL1170. Between these two different mutations, one is a synonymous mutation in Tsac_1304, which is annotated as uncharacterized protein, the other one is found in Tsac_1553, which is annotated as ferredoxin hydrogenase.

The Major Route for Pyruvate Dissimilation.

Wild type T. saccharolyticum produces 2.7 moles of C2 products (ethanol and acetate) for each mole of cellobiose consumed (since the theoretical maximum is 4, this is 68% of the theoretical maximum yield). Deletion of the primary pfor gene, pforA resulted in a dramatic decrease in growth, indicating the importance of pforA in pyruvate dissimilation. Since ethanol was still produced, it was hypothesized that pfl was partially compensating the deletion of pfor. Creation of a double deletion strain (LL1178) with both pfor and pfl deleted, produced almost no C2 products and carbon flux was redirected to lactate production. The C2 yield in this strain is −0.08 mole per mole of cellobiose consumed whereas the C3 (i.e. lactate) yield is 3.52 (88% of theoretical). The negative number for acetate in Table 5 indicates that strain LL1178 consumed part of the added sodium acetate, which was necessary for growth.

In adapted pforA deletion strains (LL1141 and LL1142), the flux through PFL was increased, which indicated by increased production of formate. If C2 products were produced exclusively via the PFL pathway, formate production and C2 yield should be equivalent on a molar basis. For strain LL1141, formate production can account for about 80% of the C2 products. For strain LL1142, formate production can account for about 84% of the C2 products (Table 5). One possible explanation for the residual C2 production is consumption of formate for biosynthesis. Another possible explanation is PFOR activity from a gene cluster other than pforA. Although PFOR activity was eliminated after deletion of pforA, adaptation resulted in the appearance of very low levels of PFOR activity that could be coming from one of the other annotated pfor genes (FIG. 2).

The Gene Encoding PFOR.

Based on data from enzyme assay and gene deletions, it appears that pforA is the gene encoding the primary PFOR activity in T saccharolyticum, which is different from the gene cluster, pforB, suggested by Shaw et al. Single deletion of the pforA cluster in wild type T. saccharolyticum completely eliminated the PFOR activity while deletions of other pfor gene clusters had no effect (FIG. 2). This result is also consistent with proteomic data for T. saccharolyticum, in which PFOR encoded by pforA is the most abundant protein among all PFOR enzymes. Enzymes encoded by other pfor gene clusters are expressed at a much lower level, at least 10 times less than that encoded by pforA. The role of these other gene clusters remains unknown.

Pyruvate Dehydrogenase and Pyruvate Decarboxylase Activity.

Neither Shaw et al. nor KEGG identified any gene encoding PDC or PDH in the genome of T. saccharolyticum. Shaw et al. also did not detect PDH or PDC activities (which has been confirmed). There are reports that PFOR can nonoxidatively decarboxylate pyruvate directly to acetaldehyde, functioning as pyruvate decarboxylase (PDC) in Pyrococcus furiosus and Thermococcus guaymasensis. Although in both cases, the acetyl-CoA production rates are higher than acetaldehyde production rates (roughly 5:1 in both organisms), the PDC side activity of PFOR is still thought to be the most likely pathway for ethanol production in hyperthermophiles. According to this ratio of PFOR activity versus PDC activity, the PDC activity should be in the order of 0.1 to 1 U/mg if the PFOR in T. saccharolyticum has this side activity. However PDC activity was not detected in cell extracts (<0.005 U/mg), so this activity is not likely to play a significant physiological role.

The existence of PDH was also examined in several other species that are closely related to T. saccharolyticum (Table 6). In some Thermoanaerobacter species, they possess all genes required to encode PDH complex, but their function and physiological roles remain to be determined experimentally.

TABLE 6
Comparison of genes involved in pyruvate metabolism and C1
metabolism between T. saccharolyticum and its relative species.

| | Enzymes | | | | | |
|---|---|---|---|---|---|---|
| | | | | Glycine | Lipoic | Lipoic |
| | | | | cleavage | acid | salvage |
| Organisms | PFOR | PDH | PFL | system | synthesis | system |
| Clostridium thermocellum | + | − | + | − | −^{b} | −^{b} |
| Clostridium clariflavum | + | − | + | − | −^{b} | −^{b} |
| Clostridium stercorarium | + | + | − | + | + | − |
| Thermoanaerobacter | + | − | + | + | − | + |
| saccharolyticum | | | | | | |
| Thermoanaerobacter tengcongensis | + | + | − | + | + | + |
| Thermoanaerobacter sp. X514 | + | + | − | + | + | + |
| Thermoanaerobacter | + | + | − | + | + | + |
| pseudethanolicus | | | | | | |
| Thermoanaerobacter italicus | + | − | − | + | + | + |
| Thermoanaerobacter mathranii | + | − | − | + | + | + |
| Thermoanaerobacter brockii | + | + | − | + | + | + |
| Thermoanaerobacter wiegelii | + | + | − | + | + | + |
| Thermoanaerobacter kivui | + | − | − | + | + | + |
| Thermoanaerobacterium | + | − | +^{a} | + | − | + |
| thermosaccharolyticum | | | | | | |
| Thermoanaerobacterium | + | − | + | + | − | + |
| xylanolyticum | | | | | | |
| Caldicellulosiruptor | + | − | − | − | + | − |
| saccharolyticus | | | | | | |
| Caldicellulosiruptor bescii | + | − | − | − | + | − |
| Caldicellulosiruptor obsidiansis | + | − | − | − | + | − |
| Caldicellulosiruptor hydrothermalis | + | − | − | − | + | − |
| Caldicellulosiruptor owensensis | + | − | − | − | + | − |
| Caldicellulosiruptor kristjanssonii | + | − | − | − | −^{b} | −^{b} |
| Caldicellulosiruptor kronotskyensis | + | − | − | − | + | − |
| Caldicellulosiruptor lactoaceticus | + | − | − | − | −^{b} | −^{b} |

^{a}T. thermosaccharolyticum DSM571 has pfl annotated whereas T. thermosaccharolyticum M0795 does not have it. It is also confirmed with protein blast using PFL protein sequence from T. saccharolyticum.
^{b}No information about lipoic acid metabolism of Clostridium thermocellum, Clostridium clariflavum, Caldicellulosiruptor kristjanssonii and Caldicellulosiruptor lactoaceticus in KEGG. The existence of lipoic acid biosynthesis and lipoic salvage system are confirmed by protein blast using Lipoyl synthase from C. bescii and lipoate protein ligase from T. saccharolyticum.

Role of pfl and C1 Metabolism.

Pyruvate formate-lyase was only expressed at low levels and was not the major route for pyruvate dissimilation in the wild type strain, It was required for growth of T. saccharolyticum grown in MTC-6 medium. The consumption of added formate and restoration of stronger growth upon addition of formate by all pfl deletions strains (Table 5) supports the hypothesis that PFL is required for biosynthesis.

It has been previously reported that PFL has an anabolic function in Clostridium species and furnishes cells with C1 units. The results presented here suggest that might also be the case in T. saccharolyticum, which belongs to class Clostridia. In Clostridium acetobutylicum, 13C labeling experiments showed that over 90% of C1 units in biosynthetic pathways come from the carboxylic group of pyruvate and are likely to be derived from the PFL reaction. Due to the defective growth of pfl deletion strains, it is likely the same case in T. saccharolyticum. In the case of C. acetobulyticum, Amador-Noguez et al. found that glycine is not formed from serine, and thus that the methyl group from serine is not transferred to tetrahydrofolate (THF) in this organism. However, in the case of T. saccharolyticum, growth of pfl deletion strains was restored by addition of serine, suggesting that C1 units are transferred from serine to THF.

Although additional glycine did not stimulate the growth of T. saccharolyticum, additional lipoic acid helped. In fact, T. saccharolyticum has all genes required for glycine cleavage system and lipoic acid salvage system. Since it does not have lipoic acid biosynthesis pathways, it required additional lipoic acid for H protein formation, which is essential for glycine cleavage system.

Among other species that we've examined, most of Thermoanaerobacter species have the glycine cleavage system and they have either lipoic acid biosynthesis or lipoic acid salvage system for H protein formation (Table 6). However, Caldicellulosiruptor species do not have PFL or glycine cleavage system. Thus, they may use serine aldolase (EC 2.1.2.1) for the supply of C1 units.

Mutations Found in Genomic Resequencing.

In one pfor deletion strain lineage (lineage 2), which includes ΔpforA-2 (strain LL1140) and its adapted descendant (strain LL1142), a SNP in lactate dehydrogenase (Tsac_0179) was found. This SNP causes an amino acid change from asparagine to serine. According to the protein structure of LDH from Bacillus stearothermophilus, which shares 48% identity with that from T. saccharolyticum, this mutation was near the catalytic site. This SNP may explain the decrease in lactate production in strains LL1140 and LL1142.

In one pfl deletion strain (LL1164) but not another (strain LL1170, a different colony from the pfl deletion experiment, see previous description), a SNP was found in the ferredoxin hydrogenase, subunit B (hfsB, Tsac_1153). A non-functional hfs gene could inhibit the PFOR reaction by preventing the oxidation of reduced ferredoxin. Shaw et al. found that deletion of the entire hfs operon resulted in a decrease in hydrogen and acetate production and increase in lactate production. Similar trends were observed for hydrogen, acetate and lactate. Shaw et al. found a slight decrease in ethanol production (22%), whereas a much larger decrease (73%) was observed here. The similarities in the patterns of fermentation data between the hfsB mutant here and the hfs deletion from Shaw et al. suggest that the hfs mutation may in fact be responsible for the change in distribution of fermentation products between strains LL1164 and LL1170.

In summary, several genes and enzymes responsible for pyruvate ferredoxin oxidoreductase and pyruvate formate lyase activities in T. saccharolyticum have been identified. The primary physiological role of PFOR appears to be pyruvate dissimilation, while the role of PFL appears to be supplying C1 units in biosynthesis. PFOR encoded by Tsac_0046 and PFL encoded by Tsac_0628 are only two routes for converting pyruvate to acetyl-CoA in T. saccharolyticum. The combination deletion of these two genes virtually eliminated pyruvate flux to acetyl-CoA, which can be seen by the shift of carbon flux to lactate production at high yield (88% of theoretical).

### Example 2 Cofactor Specificity of the Bifunctional Alcohol and Aldehyde Dehydrogenase (AdhE) in Wild-Type and Mutants of Clostridium thermocellum and Thermoanaerobacterium saccharolyticum

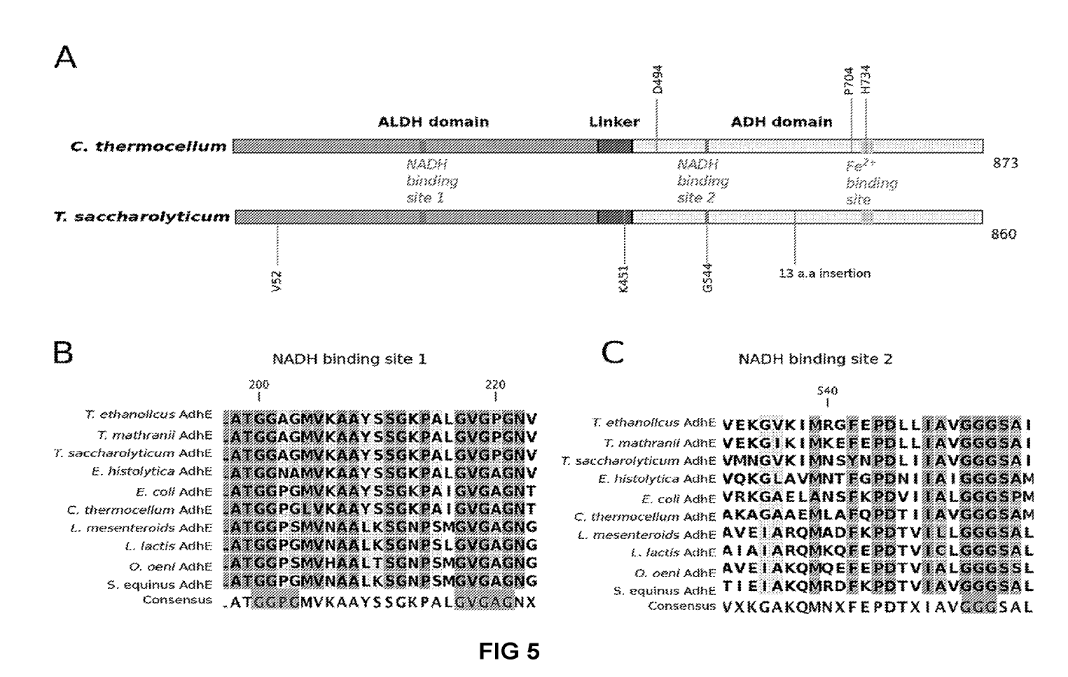

In microorganisms, fermentation of pyruvate to ethanol can proceed either with or without acetyl-CoA as an intermediate. In yeasts and Zymomonas mobilis, pyruvate is decarboxylated directly to acetaldehyde, which is then reduced to ethanol (11). In many other organisms, pyruvate is oxidatively decarboxylated to acetyl-CoA, which is reduced to acetaldehyde, which is further reduced to ethanol. This two-step conversion of acetyl-CoA to ethanol is catalyzed by one protein: a bifunctional alcohol dehydrogenase AdhE. AdhE consists of a C-terminal alcohol dehydrogenase (ADH) domain and an N-terminal aldehyde dehydrogenase (ALDH) domain: the ADH domain is usually part of the iron-containing ADH superfamily (FIG. 5) (12). AdhE is present in a variety of mesophilic and thermophilic anaerobic bacteria capable of producing ethanol as a fermentation product (13-16). AdhE has also been found in parasitic eukaryotes (17), anaerobic fungi (18) and algae (19). In all organisms investigated thus far, the deletion of adhE is associated with a loss of ethanol formation. When adhE was deleted in C. thermocellum and T. saccharolyticum, nearly 100% of ethanol production was eliminated (20), demonstrating the importance of AdhE in ethanol formation in these two organisms.

Point mutations in adhE conferring a change in cofactor specificity from NADH to NADPH in ADH activity in cell extracts have been associated with increase in ethanol tolerance in C. thermocellum (21), and increase in ethanol production in T. saccharolyticum (10). However, it has not been unequivocally established whether this cofactor specificity change must be ascribed to mutations in AdhE, as cells contain multiple alcohol dehydrogenases and measurements with cell extracts cannot distinguish between isoenzymes.

To understand the effect of these mutations, the adhE genes from six strains of C. thermocellum and T. saccharolyticum were cloned and expressed in Escherichia coli, followed by purification by affinity chromatography and enzyme activity measurement. In wild type strains of both organisms, NADH was the preferred cofactor for both ALDH and ADH activity. In high-ethanol-producing (“ethanologen”) strains, ALDH or ADH or both activities showed increased NADPH-linked activity. Interestingly, the ethanologenic C. thermocellum AdhE has acquired high NADPH-linked ADH activity while maintaining NADH-linked ADH and ALDH activity at wild-type levels. Overall, the AdhE from T. saccharolyticum ethanologenic strains had lower activities compared to wild-type, which suggests that cofactor specificity is more important for high-yield ethanol production than specific activity. Less product inhibition was observed in the AdhE from the C. thermocellum ethanol tolerant strain, which may explain the ethanol tolerance phenotype.

Plasmid and Strain Construction.

The adhE genes from strains LL1004, LL346, LL350, LL1025, LL1040, LL1049 were cloned into plasmid pEXP5-NT/TOPO (Invitrogen) with standard molecular biology techniques, generating the respective E. coli expression plasmids (Table S1). Cloning the adhE genes into pEXP5-CT/TOPO plasmids instead of pEXP5-NT/TOPO generated native AdhE proteins without His-tags. The plasmids were Sanger sequenced (Genewiz) to confirm correct insertion of the target gene, and were then transformed into chemically competent lys yr (New England Biolabs) E. coli cells. The control plasmid pNT-CALML3 (Invitrogen) was also transformed into E. coli. The resulting E. coli strains were used for protein expression. C. thermocellum strains LL1160 and LL1161 were constructed by transforming the respective integration plasmids pSH016 and pSH019 into strain LL1111 (Table 7) transformation and colony selection were carried out as previously described (22). T saccharolyticum strains LL1193 and LL1194 were constructed by transforming the respective vectors pCP14 and pCP14* into wild-type T. saccharolyticum using a natural competence based system (23) (Table 7), and transformants were selected by resistance to the antibiotic kanamycin.

TABLE 7
Strains used in this Example

| | Strain | | Accession | Source or |
|---|---|---|---|---|
| Organism | name | Description | No. ^{a} | reference |
| C. thermocellum | LL1004 | Wild-type C. thermocellum | CP002416 | DSMZ ^{c} |
| | | strain DSM 1313, low-ethanol- | | |
| | | producer ^{b} | | |
| C. thermocellum | LL346 | A.k.a “adhE* (EA)” from | SRX030164.1 | Brown et |
| | | Brown et al. (20). Evolved C. | | al. (21) |
| | | thermocellum strain, tolerant to | | |
| | | 40 g/L ethanol, have mutations | | |
| | | P704L and H734R in AdhE, | | |
| | | low-ethanol-producer. | | |
| C. thermocellum | LL350 | A.k.a “ΔhydG” from Biswas et | N/A^{f} | Biswas et |
| | | al. (48). C. thermocellum Δhpt | | al. (48) |
| | | ΔhydG strain with mutation | | |
| | | D494G in AdhE, moderate- | | |
| | | ethanol-producer ^{d}. | | |
| C. thermocellum | LL1111 | C. thermocellum Δhpt ΔadhE, | SRX744221 | Lo et al. |
| | | no ethanol production. | | (20) |
| C. thermocellum | LL1160 | LL1111 C. thermocellum strain | N/A | This study |
| | | with adhE reintroduced to the | | |
| | | original adhE locus, low- | | |
| | | ethanol-producer. | | |
| C. thermocellum | LL1161 | LL1060 with mutation D494G | N/A | This study |
| | | in AdhE, moderate-ethanol- | | |
| | | producer. | | |
| T. saccharolyticum | LL1025 | Wild-type T. saccharolyticum | CP003184 | Mai et al. |
| | | strain JW/SL-YS485, | | (3) |
| | | moderate-ethanol-producer. | | |
| T. saccharolyticum | LL1049 | A.k.a M1442. Evolved T. | SRA233073 | Mascoma |
| | | saccharolyticum Δ(pta-ack) | | Corp. |
| | | Δldh Δor796 ure metE Δeps | | |
| | | strain with mutation G544D in | | |
| | | AdhE, high-ethanol-producer ^{e}. | | |
| T. saccharolyticum | LL1040 | A.k.a strain “ALK2” from | SRA233066 | Shaw et al. |
| | | Shaw et al. (10). Evolved T. | | (10) |
| | | saccharolyticum Δldh::erm | | |
| | | Δ(pta-ack)::kan strain with | | |
| | | mutations V52A, K451N and | | |
| | | a 13-amino-acid insertion in | | |
| | | AdhE, high-ethanol-producer. | | |
| T. saccharolyticum | LL1076 | ΔadhE::(pta-ack kan), no | SRX744220 | Mascoma |
| | | ethanol production. | | Corp. |
| T. saccharolyticum | LL1193 | adhE::kan, differs from wild- | N/A | this study |
| | | type only with kan marker | | |
| | | downstream of AdhE, | | |
| | | moderate-ethanol-producer. | | |
| T. saccharolyticum | LL1194 | LL1193 with the G544D | N/A | this study |
| | | mutation in AdhE | | |

^{a} Accession numbers starting with CP refer to finished genome sequences in Genbank, accession numbers starting with SRX refer to raw sequencing data from JGI
^{b} Produces ethanol at 0-40% theoretical yield.
^{c} German Collection of Microorganisms and Cell Cultures. Leibniz-Institute, Germany.
^{d} Produces ethanol at 40-80% theoretical yield.
^{e} Produces ethanol at 80-90% theoretical yield.
^{f}Not available.

Media and Growth Conditions.

For biochemical characterization and transformation, C. thermocellum and T. saccharolyticum strains were grown anaerobically to exponential phase (OD₆₀₀˜0.5) in the appropriate medium: for C. thermocellum, CTFUD rich medium at pH 7.0 as previously described (22); for T saccharolyticum, CTFUD rich medium at pH 6.0. E. coli strains were grown in LB broth Miller (Acros) with the appropriate antibiotic. Fermentation end-products were measured using high-performance liquid chromatography as previously described (24). For end-product analysis, C. thermocellum and T. saccharolyticum strains were grown in the appropriate medium: for C. thermocellum, chemically defined MTC medium as previously described (25); for T. saccharolyticum, the MTC medium was modified as follows: thiamine was added to a final concentration of 4 mg/L, and ammonium chloride was added instead of urea. In preparation for fermentation end-product analysis, cultures were grown at 55° C. in 150 mL serum bottles with 50 mL working volume and 100 mL headspace for 72 h. Ethanol concentrations were calculated from biological duplicates.

Expression of Various adhE Genes.

500 μL of E. coli culture containing a plasmid with the adhE gene of interest was inoculated into 100 mL sterile LB broth Miller (Acros) with the appropriate antibiotic, and were grown aerobically to OD₆₀₀ 0.5 with shaking at 200 rpm at 37° C. (Eppendorf Innova 42 shaker). The E. coli strain harboring the pNT-CALML3 control plasmid (Invitrogen) was used as a negative control to measure native E. coli ADH or ALDH activity. The cultures were then transferred to sterile serum bottles, and 40 mM IPTG (Isopropyl β-D-1-thiogalactopyranoside) was used to induce protein expression. The serum bottles were then purged with nitrogen to generate an anaerobic protein expression environment, and the cells were cultured for 2 h at 37° C. before harvesting.

Preparation of Cell Extracts.

C. thermocellum, T. saccharolyticum and E. coli cultures were grown as described above. Cells were harvested by centrifugation at 3000×g for 30 min at 4° C., the supernatant was decanted and the pellet stored anaerobically at −80° C. Prior to generating cell extracts, the frozen pellets were thawed on ice and resuspended anaerobically in 0.5 mL Lysis Buffer: 1× BugBuster reagent (EMD Millipore) at pH 7.0 in phosphate buffer (100 mM) with 5 μM FeSO₄. Dithiothreitol (DTT) was added to a final concentration of 0.1 mM. For T saccharolyticum cell extracts used in ALDH activity measurements, ubiquinone-0 was added to the final concentration of 2 mM to relieve the possible inhibition on ALDH activity as previously reported (20). The cells were lysed with Ready-Lyse Lysozyme (Epicentre), and DNase I (New England Biolabs) was added to reduce viscosity. The resulting solution was centrifuged at 10,000×g for 5 min at room temperature, and the supernatant was used as cell-free-extract for enzyme assays.

Protein Purification.

The E. coli crude extracts described above were incubated at 50° C. anaerobically for 20 min to denature E. coli proteins, and the denatured proteins were separated by centrifugation. In strains LL346 and LL1040, the AdhE proteins were heat labile, and lost activity after 50° C. incubation, so these cell extracts were applied directly to the purification column without heating. E. coli cells expressing the control plasmid pNT-CALML3 were subject to the same treatment as above, and its ADH and ALDH activities before and after heat treatment were measured (Table S3). The cell extracts containing His-tagged AdhE were then subjected to anaerobic affinity column purification (Ni-NTA spin columns, Qiagen). The purification was carried out according to the Qiagen protocol “Ni-NTA Agarose Purification of 6×His-tagged Proteins from E. coli under Native Conditions” with some modifications as described below. The column was first equilibrated with Equilibrium Buffer (50 mM NaH₂PO₄, 300 mM NaCl, 5 mM imidazole, 5 μM FeSO₄, pH 7), then cell extracts were applied to the column and the column was washed twice with Wash Buffer (50 mM NaH₂PO₄, 300 mM NaCl, 50 mM imidazole, 20% ethanol, 5 μM FeSO₄, pH 7). The His-tagged AdhE was eluted by addition of 200 μL Elution Buffer (50 mM NaH₂PO₄, 300 mM NaCl, 500 mM imidazole, 5 μM FeSO₄, pH 7), this is “Eluent 1” in Table S3 and S4. Repeating this elution step sequentially generated more purified “Eluent 2” and “Eluent 3”. For C. thermocellum and T. saccharolyticum AdhE, activity was measured at various stages during purification. Electrophoresis results showed that Eluent 3 had the least amount of contaminating bands, thus Eluent 3 was used for enzyme assays. The degree of protein purity was estimated by gel densitometry using the image analysis software ImageJ, where the density of each visible gel band from Eluent 3 was plotted as peaks. The area of each peak was then integrated to generate percentages, which is an indicator of AdhE purity E. coli cell extracts with native AdhE expressed (i.e. without the His-tag) were used directly without purification.

ALDH and ADH Activity Assays.

All the ALDH activity measurements mentioned in this study refer to the reaction in the acetaldehyde-producing direction. All the ADH activity measurements mentioned in this study refer to the reaction in the ethanol-producing direction. For ADH (acetaldehyde reduction) reactions, the anaerobic reaction mixture contained 0.24 mM NADH or NADPH, 17.6 mM acetaldehyde, 1 mM DTT, 100 mM Tris-HCl, 5 μM FeSO₄ and cell extract or purified protein solution (protein amount indicated separately for each assay). The final volume was 850 μL, the assay temperature was 55° C. and the assay was started by the addition of acetaldehyde. For ALDH (acetyl-CoA reduction) reactions, the acetaldehyde in the above anaerobic reaction mixture was substituted with 0.35 mM acetyl-CoA. Decrease in absorbance at 340 nm caused by NAD(P)H oxidation was monitored by an Agilent 8453 UV-Vis spectrophotometer with Peltier temperature control. Protein concentration was determined using the Bradford method with bovine serum albumin (Thermo Scientific) as the standard. Specific activities are expressed as units per mg of protein. One unit of activity=formation of 1 μmol of product per min Specific activities in Table 8 and Table 9 are reported for at least two biological replicates. The software Visual Enzymics (SoftZymics) was used for non-linear regression to calculate the apparent Kₘ, and k_{cat} values in Table 10. k_{cat} was calculated on the basis of a molecular weight of 97-kDa (26).

TABLE 8
ADH and ALDH activities in C. thermocellum and T. saccharolyticum cell extracts

| Strain characteristics | | | | | | |
|---|---|---|---|---|---|---|
| | | Ethanol | ADH activity | | ALDH activity | |
| Strain name | Description | yield^{a} | NADH | NADPH | NADH | NADPH |
| C. thermocellum | Wild-type | 0.16 | 6.73 | 0.04 | 2.20 | 0.21 |
| LL1004 | | | | | | |
| C. thermocellum | Ethanol-tolerant | 0.11 | 0.66 | 0.38 | 0.27 | 0.05 |
| LL346 | | | | | | |
| C. thermocellum | Moderate- | 0.22 | 6.71 | 5.90 | 2.00 | 0.05 |
| LL350 | ethanol-producer | | | | | |
| C. thermocellum | adhE deletion | 0.01 | 0.10 | 0.22 | 0.05 | 0.13 |
| LL1111 | | | | | | |
| T. saccharolyticum | Wild-type | 0.26 | 7.06 | 0.95 | 0.41 | 0.05 |
| LL1025 | | | | | | |
| T. saccharolyticum | High-ethanol- | 0.43 | 0.18 | 1.10 | 0.09 | 0.50 |
| LL1049 | producer | | | | | |
| T. saccharolyticum | High-ethanol- | 0.45 | 0.08 | 1.55 | 0.08 | 0.30 |
| LL1040 | producer | | | | | |
| T. saccharolyticum | adhE deletion | 0.01 | 0.04 | 1.78 | 0.00 | 0.18 |
| LL1076 | | | | | | |

TABLE 9
Cofactor specificity of purified AdhE

| Source of AdhE | | ADH activity | | ALDH activity | |
|---|---|---|---|---|---|
| Strain name | Description | NADH | NADPH | NADH | NADPH |
| C. thermocellum LL1004 | Wild-type | 42.23 | 1.96 | 18.02 | 2.03 |
| C. thermocellum LL346 | Ethanol-tolerant | 4.02 | 0.03 | 13.88 | 0.43 |
| C. thermocellum LL350 | Moderate-ethanol-producer | 42.67 | 42.30 | 31.50 | 5.76 |
| T. saccharolyticum LL1025 | Wild-type | 17.43 | 2.43 | 10.63 | 4.46 |
| T. saccharolyticum LL1049 | High-ethanol-producer | 0.66 | 12.70 | 1.63 | 11.13 |
| T. saccharolyticum LL1040 | High-ethanol-producer | 0.01 | 12.96 | 0.07 | 5.73 |

TABLE 10
Apparent Kₘ and k_{cat} values of C. thermocellum wild-type and LL350 AdhE

| Source of | Strain | | | | | |
|---|---|---|---|---|---|---|
| AdhE | characteristics | Reaction | Substrate | Kₘ (mM) | k_{cat} (S⁻¹) | k_{cat}/Kₘ (S⁻¹M⁻¹) |
| C. thermocellum | Wild-type | ADH | Acetaldehyde | 1.3 ± 0.2 | 238 ± 8 | 1.8E+05 |
| LL1004 | | | NADH | 0.1 ± 0.02 | 302 ± 13 | 2.3E+06 |
| | | ALDH | Acetyl-CoA | 0.008 ± 0.002 | 297 ± 11 | 3.5E+07 |
| | | | NADH | 0.06 ± 0.004 | 152 ± 4 | 2.8E+06 |
| C. thermocellum | Moderate- | ADH | Acetaldehyde^{a} | 2.1 ± 0.2 | 318 ± 7 | 1.5E+05 |
| LL350 | ethanol- | | Acetaldehyde^{b} | 1.8 ± 0.2 | 246 ± 10 | 1.4E+05 |
| | producer | | NADH | 0.4 ± 0.05 | 319 ± 21 | 8.9E+05 |
| | | | NADPH | 0.08 ± 0.02 | 295 ± 18 | 3.8E+06 |
| | | ALDH | Acetyl-CoA | 0.004 ± 0.0005 | 202 ± 4 | 5.8E+07 |
| | | | NADH | 0.02 ± 0.002 | 64 ± 2 | 3.4E+06 |

Homology Modeling and Molecular Dynamics.

The homology models corresponding to the ADH domains of the AdhE from LL1004, LL346, LL350, LL1025, LL1040 and LL1049 were constructed using the bioinformatics toolkit SWISS-MODEL (Swiss Institute of Bioinformatics). The recent 2.5 Å resolution X-ray structure of the alcohol dehydrogenase of Geobacillus thermoglucosidasius (3ZDR) (12) and 1.3 Å resolution X-ray structure of the alcohol dehydrogenase from Thermatoga maritima (1O2D) (27) were used as templates for their high level of homology and presence of NADP cofactor and iron ion, respectively. The resulting structures were inspected for proper phi/psi angles. All resulting structures were submitted to molecular dynamics simulations using the program CHARMM with the CHARMM 36 force field and the TIP3P water model (28). The systems were generated via the CHARMM-GUI web server (29) and the parameters for NADP were generated by ParamChem. The structures were initially minimized in-vacuo with steepest decent for 1000 steps and then solvated in a cubic water box with a minimum of 10 Å from the edge of the box, sodium cations were added to neutralize the system. These resulting systems were minimized using steepest decent for 1000 steps followed by newton-raphson minimization for 100 steps. They were then submitted to 1-ns equilibration in the NPT ensemble at 298K and 1 bar followed by 10 ns simulation in the NVT ensemble with an integration time step of 2 fs. All simulations were conducted in duplicate with different starting seeds and analyzed using carma (30).

AdhE cofactor specificity changes from NADH to NADPH in high-ethanol-producing strains.

ADH and ALDH activity were determined in cell extracts of C. thermocellum and T. saccharolyticum strains, as well as in the affinity-purified AdhE from these strains. There were clear cofactor specificity changes from NADH to NADPH in the cell extracts (Table 8) of the C. thermocellum moderate-ethanol-producer strain (LL350) and T. saccharolyticum high-ethanol-producer strains (LL1040 and LL1049). In purified preparations of AdhE, gel densitometry results showed that the proteins were all about 80% pure (Table S5). Furthermore, negative E. coli controls all showed <0.4 U/mg specific activity for ADH and ALDH (Table S3), indicating that the contaminating proteins observed on the gel did not substantially interfere with ADH or ALDH activity measurements. With respect to cofactor preference, the results in affinity-purified AdhE enzymes followed the same trend as in cell extracts (Table 9), with the exception of the C. thermocellum ethanol-tolerant strain (LL346). In this strain, the purified AdhE was almost entirely NADH linked, but cell extracts showed small amounts of NADPH-linked activity. In all cases, the cofactor specificity change towards NADPH was much more complete in T. saccharolyticum AdhE than in C. thermocellum (Table 9). Additionally, strains exhibiting this cofactor specificity change in AdhE also generally showed increased ethanol production compared with their parent strains (Tables 8 and 9).

Because the Asp-494-Gly (D494G) mutation in the C. thermocellum moderate-ethanol-producer (LL350) AdhE enabled the enzyme to use both NADH and NADPH as cofactors, the apparent k_{cat} and Kₘ, values were measured with purified protein from both strains (Table 10). The newly acquired NADPH-linked activity in the D494G mutant resulted in an increase in catalytic efficiency for NADPH, and a decrease in catalytic efficiency for NADH. For substrates in the ALDH reaction, although they were both NADH-linked, the catalytic efficiencies were higher in the D494G mutant AdhE compared to the C. thermocellum wild-type.

Product inhibition (ethanol or NAD(P)⁺) of purified AdhE proteins from C. thermocellum and T. saccharolyticum was measured. The AdhE of the C. thermocellum ethanol-tolerant strain (LL346) was significantly different from other AdhE proteins in both ethanol and NAD(P)⁺ inhibition. It retained 98% of ADH activity and 92% of ALDH activity in the presence of 2.35 mM NAD⁺. Interestingly, it showed a 2-fold increase in ADH activity in the presence of 1 M ethanol.

Effect of AdhE Mutations on Ethanol Production.

The physiological effect of two selected point mutations was investigated by re-introducing those mutations into either C. thermocellum or T. saccharolyticum. For C. thermocellum, the D494G mutation was chosen. This mutation cannot be introduced directly into the wild-type strain (due to limits of existing genetic tools), so instead adhE was deleted (strain LL1111) and replaced by the D494G mutant adhE (strain LL1161). A control strain (LL1160) was made by re-introduction of the wild type adhE into strain LL1111. Fermentation of 14.7 mM cellobiose resulted in ethanol production of 15.2 mM for strain LL1160 and 26.1 mM for strain LL1161, a 1.7-fold increase.

For T. saccharolyticum, the AdhE G544D mutation was chosen. In this organism, the mutation could be introduced directly into the wild type strain, although a kanamycin (kan) antibiotic resistance marker had to be added downstream of adhE. The resulting strain was LL1194. A control strain (LL1193) was made by inserting only the kan marker downstream of adhE. Fermentation of 14.7 mM cellobiose resulted in ethanol production of 23.4 mM for strain LL1193 and 34.5 mM for strain LL1194, a 1.5-fold increase.

AdhE Protein Structure Prediction.

To understand the impact of mutations on cofactor specificity homology modeling and docking were performed. The average structure of the ADH domains from wild type and D494G mutants of the C. thermocellum AdhE were compared to identify potential explanations for the switch in cofactor specificity. In wild-type C. thermocellum AdhE, the Asp-494 interferes with the 2′-phosphate group of NADPH because of electrostatic repulsion (both negatively charged) and steric hindrance. Molecular dynamics simulation was conducted to compare the average structures of the six different ADH domains including the previously mentioned mutant D494G to evaluate if the observation from homology modeling and docking were correct. The conformation of NADPH in the binding pocket of the ADH domain varied in the AdhE mutants. In the case of C. thermocellum, the behavior of NADPH is similar in wild-type C. thermocellum (LL1004) AdhE and the ethanol-tolerant C. thermocellum (LL346) AdhE. In the C. thermocellum moderate-ethanol-producer (LL350) AdhE, the D494G mutation changed NADPH binding significantly as mentioned above. A similar trend was observed in the case of T. saccharolyticum AdhE where the mutations in the high-ethanol-producers LL1049 and LL1040 seem to change the conformation of NADP in the binding pocket.

Primary Structure of AdhE.

The ADH and ALDH domains of AdhE are highly conserved and connected by a linker sequence which contains a putative NADH binding domain (26, 31-33). There is a disagreement in the literature on the number of NADH binding sites in AdhE. Some studies predict a single NADH binding site located within or near the linker region of AdhE (13, 31-35), suggesting that the ADH and ALDH domains share one nicotinamide binding site. Other studies predict an additional NADH binding site in the ALDH domain (19, 26, 36, 37). Fungal AdhE enzymes have been shown to have three putative NADH binding sites (18). Here, the analysis was focused on glycine rich regions and it relied on structural information from the homology models or closely related structural homologs. In both C. thermocellum and T. saccharolyticum AdhE proteins, two strong NADH binding sites were found (FIG. 5A) and an additional putative nucleotide binding region with a GXGXXG motif in the linker region between the ADH and ALDH domains, which has been reported in previous studies as a potential recognition locus (13, 18, 19, 26, 31-37). This putative binding region within the linker is almost identical to one identified in another iron-dependent alcohol dehydrogenase from E. coli (38). In this study, the glycine at the center of the locus was mutated but only resulted in a marginal loss of NAD⁺ binding. However, mutations in the other glycine rich locus with the motif GGG located in the ALDH domain resulted in complete loss of NAD⁺ binding (38), indicating that the GGG motif is important in NAD⁺ binding. The GXGXXG motif located in the linker is conserved in several ADHE enzymes but do not seem to contribute to nucleotide binding directly but might be important in nucleotide channeling or recognition before entering the binding pocket. The NADH binding site in the ALDH domain appears to be a combination of a conventionally accepted binding motif GXGXG and another glycine rich helix turn (FIG. 1B). The NADH binding site in the ALDH domain has also been suggested to have acetyl-CoA binding abilities (26). High level of conservation for both strong binding sites across different organisms (FIGS. 1B and 1C) was observed. The prediction of two binding sites (one in each domain) agrees with the observation that the D494G mutant AdhE gained NADPH specificity for ADH activity but not for ALDH activity. If the D494G mutant AdhE shared a single NADH binding site in the linker region, then one would expect to find NADPH cofactor specificity in ALDH activity as well.

Cofactor specificity change from NADH to NADPH is linked to higher ethanol production.

Most bifunctional AdhE enzymes investigated are NADH-linked, with some exceptions. The Thermoanaerobacter mathranii AdhE showed a small amount of NADPH-linked activity in addition to NADH-linked activity for both ADH and ALDH (31), and the Thermoanaerobacter ethanolicus JW200 AdhE showed NADH-linked ALDH activity and small amounts of NADPH-linked ADH activity (33). In all strains investigated in this study, higher ethanol production was linked to a cofactor specificity change from NADH to NADPH. In some cases this occurred by relaxation of cofactor specificity (C. thermocellum strain LL350). In other cases it occurred by eliminating most of the NADH linked activity (T. saccharolyticum strains LL1040 and LL1049). Furthermore, when mutations that were previously shown to increase NADPH-linked ADH activity were re-introduced into wild-type C. thermocellum and T saccharolyticum AdhE, ethanol production increased in the resulting strains (LL1161 and LL1194). This result suggests that the AdhE mutation was, in fact, the cause of the increased ethanol production.

Cofactor specificity change from NADH to NADPH is linked to higher ethanol production.

Thus far, most bifunctional AdhE enzymes investigated are NADH-linked: however, there are some exceptions. The Thermoanaerobacter mathranii AdhE showed a small amount of NADPH-linked activity in addition to NADH-linked activity for both ADH and ALDH (31), and the Thermoanaerobacter ethanolicus JW200 AdhE showed NADH-linked ALDH activity and small amounts of NADPH-linked ADH activity (33). In all strains investigated in this study, higher ethanol production was linked to a cofactor specificity change from NADH to NADPH. In some cases this occurred by relaxation of cofactor specificity (C. thermocellum strain LL350). In other cases it occurred by eliminating most of the NADH linked activity (T. saccharolyticum strains LL1040 and LL1049). Furthermore, when mutations that were previously shown to increase NADPH-linked ADH activity were re-introduced into wild-type C. thermocellum and T. saccharolyticum AdhE, ethanol production increased in the resulting strains (LL1161 and LL1194). This suggests that the AdhE mutation was, in fact, the cause of the increased ethanol production.

Brown et al. (21) have reported that mutations in the adhE gene of C. thermocellum LL346 (P704L and H734R) are the sole basis for the alcohol tolerance of this mutant. As the mutations coincided with a change from NADH-linked to NADPH-linked ADH activity in cell-free extracts, they concluded that these mutations are responsible for a change of the cofactor specificity from NADH to NADPH in the ADH part of AdhE. An increase in NADPH-linked ADH activity was observed in the cell-free extracts of LL346 compared to wild-type (Table 8), but in assays done with the purified AdhE from LL346, nearly 100% of the activity for both ADH and ALDH was NADH-linked (Table 9). This observation changes the interpretation of the effect of the mutation, and suggests that its effects are reducing enzyme activity instead of changing cofactor specificity. It is also possible that the small increase of NADPH-linked ADH activity observed in the cell extracts of LL346 is a result of another enzyme.

The driving force for the change in cofactor specificity towards NADPH in strains LL350, LL1040 and LL1049 remains to be elucidated. NADPH is the reducing equivalent for anabolism whereas NADH is the reducing equivalent in anaerobic catabolism. Apparently these strains use NADPH for both anabolic and catabolic processes. This phenomenon may be related to changes in the fluxes of NADH and NADPH generation elsewhere in metabolism. There are several possible sources in C. thermocellum and T. saccharolyticum that can provide NADPH for ethanol production. The nfnAB genes, which are present in both C. thermocellum and T. saccharolyticum, encode the NfnAB complex that catalyzes the reaction: 2NADP⁺+NADH+Ferredoxin_{red}²⁻+H⁺→2NADPH+NAD⁺+Ferredoxinₒₓ (39). Other T. saccharolyticum enzymes that generate NADPH for catabolic purposes include the glucose-6-phosphate dehydrogenase and the phosphogluconate dehydrogenase. These T. saccharolyticum genes are both highly expressed (40). Although C. thermocellum does not have the above two enzymes present in the pentose phosphate cycle, the malic enzyme in C. thermocellum, which catalyzes the formation of pyruvate from L-malate and generates NADPH, is very active (24).

Enzymes in the T. saccharolyticum ethanol production pathway.

Although the T. saccharolyticum LL1040 and LL1049 strains are able to produce ethanol at high yield, purified AdhE proteins from these mutant strains showed lower ADH activity (Table 9), and the ADH activity in cell extracts of LL1040 and LL1049 is similar to that in the T. saccharolyticum adhE deletion strain LL1076 (Table 8). This suggests that the T. saccharolyticum high-ethanol-producers do not largely rely on the ADH activity from AdhE for ethanol production, and that another alcohol dehydrogenase may be the main ADH in these strains. The cell extract activity measurements in Table 8 suggests that this other ADH is NADPH-linked and may have higher ADH activity than AdhE. It has been reported that an NADPH-linked primary alcohol dehydrogenase AdhA is present in the Thermoanaerobacter species, and may be part of the ethanol production pathway (41, 42). Sequence analysis shows T. saccharolyticum JW/SL-YS485 has a gene (Tsac_2087) encoding an alcohol dehydrogenase that is 86% identical (at the protein level) to the T. mathranii and T ethanolicus AdhA. Other reported NADPH-linked alcohol dehydrogenases involved in ethanol production include the AdhB enzyme, such as the secondary alcohol dehydrogenase reported in T. ethanolicus 39E (43). However, sequence analysis showed that T. saccharolyticum does not possess an adhB gene. Therefore AdhA may be responsible for the observed NADPH-linked ADH activity in T. saccharolyticum cell extracts, and also may be important in ethanol production in the T. saccharolyticum high-ethanol-production strains LL1040 and LL1049.

Product Inhibition of AdhE.

It has been reported that low amounts of NAD⁺ and ethanol inhibit ADH activity in cell extracts of C. thermocellum (44). High inhibition by NAD(P)⁺ in purified AdhE proteins in C. thermocellum was observed (at least 70% activity was inhibited), with the exception of LL346, in which inhibition by NAD⁺ was less than 10%. Another unexpected property of the AdhE from strain LL346 was increased activity in the presence of ethanol. This property may explain the increased ethanol tolerance of this strain (21). A similar phenomenon has been observed with the Z. mobilis ZADH-2 enzyme, which was also stimulated by ethanol (45). The authors proposed ethanol-induced acceleration of NAD⁺ dissociation as a mechanism for the observed activation by ethanol, because nicotinamide dissociation is presumed to be the rate-limiting step in most dehydrogenases.

AdhE Cofactor Specificity at the Molecular Level.

Several factors may explain the changes in cofactor specificity described in the C. thermocellum moderate-ethanol-producer AdhE (from strain LL350). It is clearly not energetically favorable to accommodate the extra 2′-phosphate group in the wild type C. thermocellum AdhE because of the negative charge of Asp-494. This 2′-phosphate group is absent in NADH, which may in fact be stabilized by hydrogen bonding interactions with this residue. This evidence suggests that Asp-494 is important in distinguishing nicotinamide cofactors as previously described. As shown in FIG. 2, the substitution of Asp-494 to glycine removes the interference between Asp-494 and NADPH thus enabling the ADH to use both NADH and NADPH as a cofactor. The low Kₘ value for NADPH in the D494G mutant AdhE (Table 10) agrees with the structural prediction, as it suggests that this mutation resulted in an increase in affinity of the enzyme to NADPH. Aspartic acid residues have been shown to play an important role regulating the binding of NADH over NADPH and are potential targets for mutations to change cofactor specificity. For example, the mutation D38N in the NADH recognition motif of an NADH dependent ADH from a Drosophila allowed the enzyme to use both NADH and NADPH (46). A similar study was conducted on an alcohol dehydrogenase yielding the same results (38). The positions of these aspartic acids are almost identical to that of D494 in wild-type C. thermocellum (LL1004) AdhE.

Regarding LL346, the mutations would likely lead to a loss of enzymatic activity in AdhE. Even though the LL346 mutations H734R and P704L both occurred in the ADH domain, the ALDH activity may also be affected. The H734R mutation has been studied in E. histolytica AdhE (a.k.a EhADH2), where it resulted in reduced ADH and ALDH activity (26). Their results suggested that alterations in the ADH domain, especially within the putative iron-binding domain where H734R resides, could affect ALDH domain activity. Helical assemblies of AdhE proteins named “spirosomes” have been observed in other organisms (12, 26, 35, 47), and the formation of such structures has been suggested to influence enzyme activity. The formation of this quaternary structure offers another explanation as to why mutations in one domain of AdhE may impact the activity of the other domain.

In the wild type T. saccharolyticum AdhE (from strain LL1025), Asp-486 is the equivalent of Asp-494 in the C. thermocellum AdhE and as mentioned above probably selectively mediates the binding of NADH over NADPH. The mutation in LL1049 replaces a glycine residue by a charged aspartic acid across from Asp-486 and the 2′-phosphate group of NADPH appears sandwiched between these two amino acid residues. There are several hydrogen bounds shared between this phosphate group and the two aspartic acids that could help relieve their overall repulsion based on their respective charges. In the case of the LL1040 variant, there is a large loop of 13 amino acids introduced in the ADH domain, and given its flexibility and close proximity to the NADH binding site in the linker sequence, could induce subtle changes to the binding site that would result in the observed cofactor specificity change.

Regarding cofactor change in the ALDH domain of the LL1040 and the LL1049 mutants, this domain either possess a mutation far from the NADH binding site (LL1040) or lacks such a mutation (LL1049). It is possible that spirosome formation (12, 26, 35, 47) not only influences enzyme activity, but also affects cofactor specificity: thus cofactor changes in the ADH domain may cause cofactor changes in the ALDH domain through the formation of such superstructures.

In summary, the AdhE from T. saccharolyticum ethanologenic strains had lower activities compared to wild-type, which suggests that cofactor specificity is more important for high-yield ethanol production than specific activity. Also, less product inhibition was observed in the AdhE from the C. thermocellum ethanol tolerant strain, which may explain the ethanol tolerance phenotype.

### Example 3 Deletion of nfnAB in Thermoanaerobacterium saccharolyticum and its Effect on Metabolism

In this Example, experiments were performed to (1) determine the physiological role of the NfnAB complex in T. saccharolyticum and (2) whether this role change in strains that have been engineered for high-yield ethanol production.

To answer these questions, targeted gene deletion, heterologous gene expression, biochemical assays, and fermentation product analysis were used to understand the role of the NfnAB complex in anaerobic saccharolytic metabolism.

Materials and methods used in this Example are described below. Chemicals, Strains, and Molecular techniques.

All chemicals were of molecular grade and obtained from Sigma-Aldrich (St. Louis, Mo., USA) or Fisher Scientific (Pittsburgh, Pa., USA) unless otherwise noted. A complete list of strains and plasmids is given in Table 11. Primers used for construction of plasmids and confirmation of nfnAB manipulations are listed in Supplemental Table 1. Transformation and deletion of nfnAB in T. saccharolyticum (Tsac_2085-6) was accomplished with plasmid pMU804 (3). Plasmid pMU804 was generated by digesting plasmid pMU110 (8) with BamHI and Xhol (New England Biolabs, Beverly, Mass., USA) and using primers to amplify ˜800 bp of regions flanking Tsac_2085-6 as well as a Kan^{r} gene. The resulting PCR products and plasmid digest were ligated together using yeast gap repair (9). Plasmids were extracted from yeast and transformed into E. coli and screened for the correct insert by restriction digest (9). For complementation of nfnAB under control of a xylose inducible system into strain LL1220, ˜500 bp of the xynA upstream region, nfnAB, an End gene, and −500 bp downstream region of xynA were ligated together in that order using overlapping primers and Gibson assembly (New England Biolabs). The resulting fragment was cloned into a pCR-Blunt II vector (Life Technologies, Carlsbad, Calif., USA) for ease of propagation of the fragment. A colony was screened, sequenced, and found to have the correct fragment. This colony was named pJLO31.

Media and Growth Conditions.

All strains were grown anaerobically at 55° C., with an initial pH of 6.3. Bacteria for transformations and biochemical characterization were grown in the modified DSMZ M122 rich media containing 5 g/L cellobiose and 5 g/L yeast extract as previously described with minor modifications (10). To prepare cell extracts, cells were grown to an OD₆₀₀ of 0.5-0.8, separated from media by centrifugation and used immediately or stored anaerobically in serum vials at −80° C. as previously described (4, 5). For quantification of fermentation products on cellobiose, strains were grown shaking in 150 mL glass bottles with 50 mL working volume in MTC defined media on 5 g/L (14.4 mM or 0.72 mmoles) cellobiose, as previously described (11) with the following modifications for T. saccharolyticum: urea was replaced with ammonium chloride, and thiamine hydrochloride was added to a final concentration of 4 mg/L. Growth media for ΔpyrF strains was supplemented with 40 mg/L uracil. For fermentation and biochemical nfnAB complementation experiments on xylose, strains were grown in 35 mL tubes on DSMZ M122 media in 5 g/L xylose. Samples were taken during mid log phase for biochemical assays, while growth was allowed to proceed for 96 hours for fermentation product quantification. All fermentation experiments were performed in triplicate.

Heterologous Expression of T. saccharolyticum nfnAB.

The putative T. saccharolyticum nfnAB operon was cloned into a pEXP5-NT TOPO expression vector (Life Technologies) and transformed into E. coli DH5α cells (Life Technologies). Plasmids were sequenced using primers provided with the kit, and a plasmid with the correct sequence was named pJLO30. For expression, pJLO30 was transformed into E. coli T7 Express lysY/I^{q} cells (New England Biolabs), grown and induced as previously described (6), scaled down to 150 mL bottles. Briefly, cells were grown on tryptone-phosphate broth in shaking incubators for 20 hours. After 20 hours stirring was stopped, and cultures were induced with IPTG (isopropyl β-D-thiogalactopyranoside). Cysteine (0.12 g/L), ferrous sulfate (0.1 g/L), ferric citrate (0.1 g/L), ferric ammonium citrate (0.1 g/L) were added to enhance iron-sulfur cluster synthesis. Cells were incubated for another 20 hours at 27° C., then separated from media by centrifugation and stored anaerobically in serum vials at −80° C. until used.

Preparation of Cell-Free Extracts.

All steps were performed in a Coy (Grass Lake, Mich., USA) anaerobic chamber to maintain anoxic conditions. Cells were lysed by 20 minute incubation in an anaerobic buffer containing 50 mM morpholinepropanesulfonic (MOPS) sodium salt (pH 7.5), 5 mM dithiothreitol, 1 U/100 μL Ready-Lyse lysozyme (Epicentre Biotechnologies, Madison, Wis., USA), and 1 U/100 μL DNase I (Thermo Scientific, Waltham, Mass., USA). Lysed cells were centrifuged for 15 minutes at 12,000 g, the pellet was discarded, and the supernatant was kept as cell-free-extract. Protein from the resulting cell-free extract was measured using Bio-Rad (Hercules, Calif., USA) protein assay dye reagent with bovine serum albumin (Thermo Scientific) as a standard.

Biochemical Assays.

All biochemical assays, manipulations, and polyacrylamide gel electrophoresis (PAGE) were performed in a Coy anaerobic chamber with an atmosphere of 85% N₂, 10% CO₂, and 5% H₂ at 55° C. Oxygen was maintained at <5 ppm by use of a palladium catalyst. Solutions used were allowed to exchange gas in the anaerobic chamber for at least 48 hours prior to use.

Triphenyltetrazolium chloride (TTC) reduction with NADPH (NFN activity).

Assaying cell-free extract for NFN activity using TTC was based on the method of Wang et al (6), with minor modifications for use in a 96-well plate. Changes in absorbance were measured in a Powerwave XS plate reader (Biotek, Winooski, Vt., USA) at 55° C. as previously described (11). The assay mixture contained 50 mM MOPS sodium salt (pH 7.5), 10 mM β-mercaptoethanol, 12 μM FAD, 0.5 mM NADP⁺, 40 mM glucose-6-phosphate, 0.2 U of glucose-6-phophate dehydrogenase (Affymetrix, Santa Clara, Calif., USA), 0.4 mM TTC, and 2 mM NAD⁺ as needed. Cell-free extract was combined with 200 μl of reaction mixture with and without NAD⁺. TTC reduction was followed for 15 minutes at 546 nm (ε=9.1 mM⁻¹ cm⁻¹).

Benzyl viologen reduction with NADPH on native PAGE (FNOR activity).

Separating proteins anaerobically using native PAGE was based on the method of Fournier et al (12) with minor modifications. 20 minutes prior to loading cell free extract, 20 μL of 2 mM sodium dithionite (DT) was loaded into each well of a 4-20% non-denaturing polyacrylamide gel (Bio-Rad). Cell-free extract containing approximately 0.1 mg protein was then loaded into the gel using a 5× native loading buffer containing 62.5 mM Tris-HCl (pH 6.8), 40% glycerol, and 0.01% bromophenol blue. The gel was run at 200V for 80 minutes in running buffer containing 25 mM Tris-HCl (pH 8.5) and 192 mM glycine. After electrophoresis, the gel was placed in prewarmed 55° C. enzyme assay buffer containing 50 mM MOPS (pH 7.5) and 8 mM benzyl viologen (BV). DT was added until the solution reached an OD at 578 nm of 0.01-0.1. The reaction was started with the addition of 1.5 mM NADPH and incubated for 15 minutes. Bands of reduced BV were fixed by adding 24 mM TTC.

Benzyl viologen: NAD(P)H oxidoreductase activity of cell-free extracts (FNOR activity).

Benzyl viologen: NAD(P)H oxidoreductase activity was measured as previously described (4) with minor modifications using the following conditions: 50 mM MOPS (pH 7.5), 0.5 mM DTT, 1 mM BV, and 0.2 mM NAD(P)H. DT was added until the solution reached an OD at 578 nm of 0.01-0.1 (ε=7.8 mM⁻¹ cm⁻¹). Changes in absorbance were measured with an Agilent Technologies 8453 UV-Vis spectrophotometer (Santa Clara, Calif., USA).

Alcohol and Aldehyde Dehydrogenase Activity of Cell-Free Extracts.

Alcohol dehydrogenase (ADH) and aldehyde dehydrogenase (ALDH) activity was measured as previously described (5). ADH and ALDH was monitored by measuring NAD(P)H oxidation at 340 nm (ε=6,220 M⁻¹ cm⁻¹). For ADH measurements, the reaction mixture contained 100 mM Tris-HCl buffer (pH 7.0), 5 μM FeSO₄, 0.25 mM NAD(P)H, 18 mM acetaldehyde, and 1 mM dithiothreitol (DTT). For ALDH measurements, the reaction mixture contained 100 mM Tris-HCl buffer (pH 7.0), 5 μM FeSO₄, 0.25 mM NAD(P)H, 1.25 mM acetyl-CoA, 1 mM DTT, and 2 mM dimethoxy-5-methyl-p-benzoquinone.

Glucose-6-phosphate dehydrogenase and Isocitrate dehydrogenase activity of cell-free extracts.

Glucose-6-phosphate dehydrogenase (13) and isocitrate dehydrogenase (14) activity was measured as previously described, following the reduction of NADP⁺ at 340 nm. For glucose-6-phosphate dehydrogenase measurements, the reaction mixture contained 100 mM Tris-HCl buffer (pH 7.5), 2.5 mM MnCl₂, 6 mM MgCl₂, 2 mM glucose-6-phosphate, 1 mM DTT, and 1 mM NADP⁺. For isocitrate dehydrogenase activity, the reaction mixture contained 25 mM MOPS (pH 7.5), 5 mM MgCl₂, 2.5 mM MnCl₂, 100 mM NaCl, 1 mM DTT, 1 mM DL-isocitrate, and 1 mM NADP⁺.

Analytical Techniques.

Fermentation products in the liquid phase (cellobiose, xylose, ethanol, lactate, acetate, and formate) were measured using a Waters (Milford, Mass., USA) HPLC with a HPX-87H column as previously described (11). H₂ was determined by measuring total pressure and H₂ percentage in headspace. Headspace gas pressure in bottles was measured using a digital pressure gauge (Ashcroft, Stratford, Conn., USA). Headspace H₂ percentage was measured using a gas chromatograph (Model 310; SRI Instruments, Torrence, Calif., USA) with a HayeSep D packed column using a thermal conductivity detector with nitrogen carrier gas. Pellet carbon and nitrogen was measured with a Shimadzu TOC-V CPH elemental analyzer with TNM-1 and ASI-V modules (Shimadzu Corp., Columbia, Md., USA) as previously described (15).

Genetic Manipulation of nfnAB.

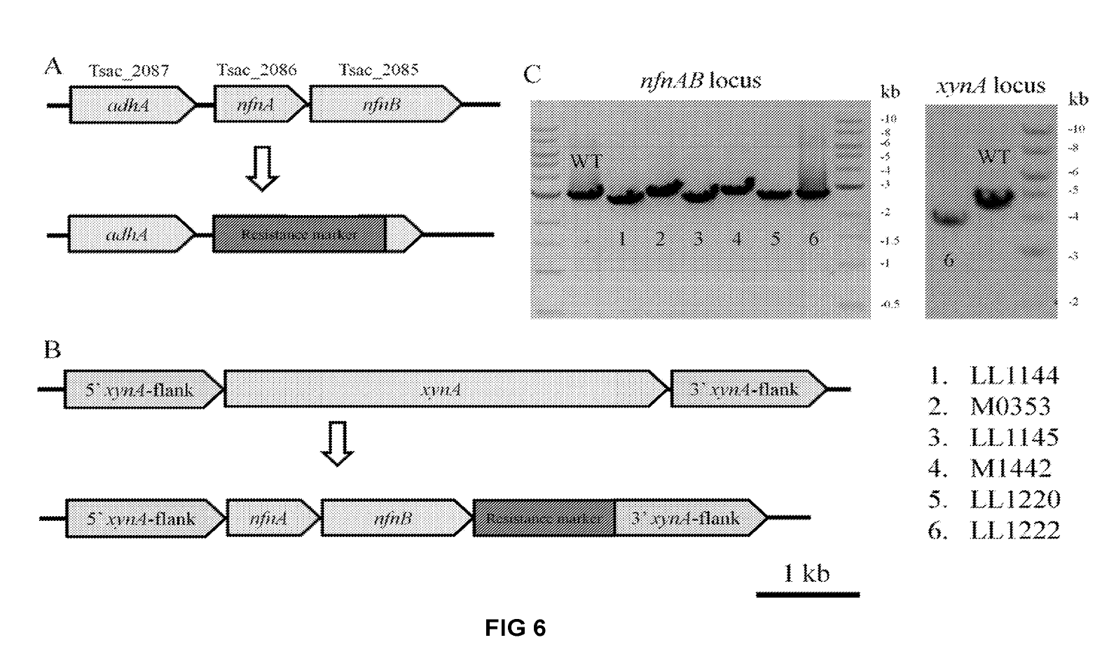

To determine the role of nfnAB in metabolism, nfnAB was deleted in the following T. saccharolyticum strains: wild-type JW/SL-YS485, M0353 (16), and M1442 (17). The resulting ΔnfnAB::Kan^{R} strains were LL1144, LL1145, and LL1220, respectively (Table 11). M0353 and M1442 are strains that had previously been engineered for improved ethanol yield. In strain LL1220 (M1442 ΔnfnAB), the nfnAB deletion was complemented with nfnAB under control of the xynA promoter to generate strain LL1222. The xynA promoter allows nfnAB to be conditionally expressed in the presence of xylose (18). Strategies for manipulation of nfnAB and PCR gels confirming nfnAB genetic modifications in T. saccharolyticum are shown in FIG. 6.

TABLE 11
Strains or plasmids described in this document

| Strain or Plasmid | Description | Reference or Source |
|---|---|---|
| Strains | | |
| T. saccharolyticum | | |
| JW/SL-YS485 | Wild-type | Gift from Juergen Wiegel |
| LL1144 | JW/SL-YS485 ΔnfnAB::Kan^{r} | This work |
| M0353 | Δpta Δack Δldh ΔpyrF (aka LL1174) | 16 |
| LL1145 | M0353 ΔnfnAB::Kan^{r} | This work |
| M1442 | Δpta Δack Δldh adhE^{G544D} (aka LL1049) | 17 |
| LL1220 | M1442 ΔnfnAB::Kan^{r} | This work |
| LL1222 | LL1220 ΔxynA::nfnAB Ery^{r} | This work |
| E. coli | | |
| T7 Express lysYll^{q} | Used for heterologous protein expression | New England Biolabs |
| DH5α | Used for plasmid screening and propagation | New England Biolabs |
| Plasmids | | |
| pMU804 | Disrupts nfriAB with kanamycin resistance gene | This work |
| pJLO30 | pEXP5-NT TOPO expressing T. saccharolyticum nfnAB | This work |
| pJLO31 | Inserts nfnAB with erythromycin resistance gene in xynA region | This work |

NAD⁺-stimulated TTC reduction with NADPH in cell free extracts (NFN activity).

To confirm NFN activity and biochemical changes associated with nfnAB deletion, NAD⁺ stimulated reduction of TTC with NADPH was measured in cell-free extracts of T. saccharolyticum (Table 12). Increased TTC reduction in the presence of NAD⁺ is characteristic of NFN activity (6). Cell-free extracts of T. saccharolyticum strain JW/SL-YS485 (wild type for nfnAB) showed NAD⁺ stimulated reduction of TTC with NADPH, which disappeared in strain LL1144 (JW/SL-YS485 ΔnfnAB).

TABLE 12
Specific activities of NfnAB from T. saccharolyticum
Specific activity (umol · min⁻¹ · mg⁻¹ protein) SD* in parenthesis

| | | T. saccharolyticum strains | |
|---|---|---|---|
| | | JW/SL-YS485 | LL1144 |
| | Genotype | WT | ΔnfnAB::Kan^{r} |
| Reaction | | | |
| NADPH → TTC | (−NAD⁺) | 0.34(0.10) | 0.20(0.08) |
| | (+NAD⁺) | 0.69(0.15) | 0.13(0.10) |
| NfnAB activity | | Yes | No |

Alternative NADPH Generating Reactions.

With the loss of NFN activity, which is a potential source of NADPH in T. saccharolyticum, it would be of interest to determine how NADPH was generated for biosynthetic reactions. Thus, the presence of a functional oxidative pentose phosphate pathway in JW/SL-YS485 was tested by assaying for glucose-6-phophate dehydrogenase and found significant activity (0.35 U/mg protein). Significant NADPH-linked isocitrate dehydrogenase activity (0.67 U/mg protein) was found, another NADPH generating reaction.

Native PAGE Assay (FNOR Activity).

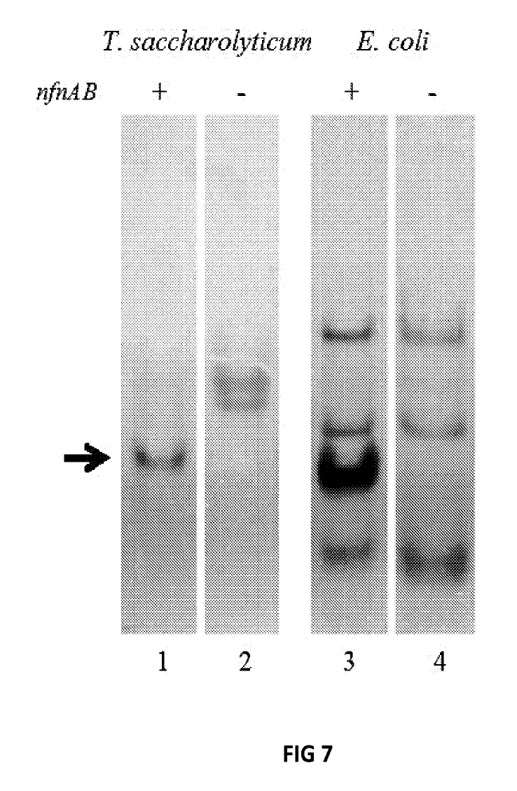

Since cell-free extracts contain multiple redox-active enzymes, methods to determine the NfnAB activity of specific proteins within the cell-free extract were tested. Native PAGE has been used to assay hydrogenase activity with redox-sensitive viologen and TTC dyes (12). This principle was applied to identify the presence of NfnAB, since the NfnAB complex from C. kluyverii was shown to strongly catalyze BV reduction with NADPH (6). Cell-free extracts of T. saccharolyticum JW/SL-YS485 (wild type for nfnAB) and LL1144 (JW/SL-Y5485 ΔnfnAB), and E. coli heterologously expressing T. saccharolyticum nfnAB were separated by PAGE in the anaerobic chamber. The PAGE gel was then incubated in prewarmed enzyme activity buffer containing NADPH and BV. Bands indicating BV reduction, consistent with the presence or absence of nfnAB, were identified in both the T saccharolyticum and E. coli tested strains, marked by an arrow (FIG. 7). The bands in the upper portion of the gel in T. saccharolyticum appeared before NADPH was added to the buffer and are suspected to be hydrogenases.

Alcohol dehydrogenase and Ferredoxin: NAD(P)H oxidoreductase (FNOR) activity of cell-free extracts.

Cell-free extracts were assayed for ADH, ALDH, and FNOR activity (Table 13). Presence of nfnAB genes corresponds to high NADPH-linked BV activity in all tested strains, while deletion of nfnAB genes corresponds to low NADPH-linked BV activity (>0.07), showing that nfnAB is responsible for most of the NADPH-linked BV reduction in T. saccharolyticum.

Interestingly, NADH-linked BV activity remained high in all strains regardless of whether or not nfnAB was present, suggesting nfnAB is not the sole FNOR in T. saccharolyticum. ADH activity was primarily NADH-linked in strains JW/SL-YS485 (wild type for nfnAB), LL1144 (JW/SL-YS485 ΔnfnAB), and M0353 (Δpta Δack Δldh ΔpyrF), and almost exclusively NADH-linked in LL1145 (M0353 ΔnfnAB). In contrast, M1442 (Δpta Δack Δldh adhE^{G544D}) and LL1220 (M1442 ΔnfnAB) has primarily NADPH-linked ADH activities.

Fermentation Products of Deletion Strains.

After biochemical characterization of activities from cell-free extracts, the effect of nfnAB deletions on fermentation product distribution was measured (Table 14). Deletion of nfnAB in the wild-type strain (resulting in strain LL1144) had very little effect on fermentation products, with the exception of H₂, which showed a 46% increase and acetate, which showed a 21% increase.

Deletion of nfnAB in the M0353 (Δpta Δack Δldh ΔpyrF) ethanologen strain (resulting in strain LL1145) had no substantial effect on fermentation product production.

Deletion of nfnAB in the M1442 (Δpta Δack Δldh adhE^{G544D}) ethanologen strain (resulting in strain LL1220), however, gave different results. Ethanol yield, which had been about 80% of theoretical in M1442, was reduced to about 30% of theoretical in LL1220. H₂ production increased ten-fold and biomass was reduced by about half.

Complementation of nfnAB in Strain LL1220.

The role of nfnAB in ethanol production was further confirmed by a complementation experiment at the xynA locus. This locus had previously been used for xylose inducible expression of genes (18). The xynA gene was replaced with nfnAB under the control of the xynA promoter in strain LL1220 (M1442 ΔnfnAB) to make strain LL1222 (LL1220 ΔxynA::nfnAB Ery^{r}) (FIG. 1B). Strains M1442, LL1220, and LL1222 were grown in M122 media with 5 g/L xylose (both as a carbon source and to induce expression of nfnAB) and tested for ethanol formation and NADPH:BV reduction (Table 5). Significant NADPH:BV activity was found in both M1442 and LL1222, but not LL1220, showing that nfnAB was expressed in LL1222. Less NADPH:BV activity was detected in LL1222 versus M1442, suggesting that nfnAB complementation was incomplete.

Next, the ethanol formation of M1442, LL1220, and LL1222 was examined M1442 and LL1222 both had high yields of ethanol from consumed xylose (109% and 69% respectively), while LL1220 had a much lower yield of ethanol (19%), suggesting that nfnAB was important to ethanol formation in M1442. Xylose consumption was impacted in both mutant strains of LL1220 and LL1222, as strains were unable to consume the provided xylose within 96 hours, although strain LL1222 consumed more xylose than strain LL1220.

The purpose of this experiment was to understand the physiological role of nfnAB in T. saccharolyticum and several mutants engineered for increased ethanol production. In the wild-type strain, NAD⁺ stimulated TTC reduction with NADPH was observed, which is a reaction indicative of its function as a bifurcating enzyme. A new assay was demonstrated for detecting the NfnAB complex using the native PAGE based assay, which was confirmed by deletion in T. saccharolyticum and heterologous expression in E. coli, thus linking the coding region annotated as Tsac_2085-6 with this activity. Furthermore, this activity is necessary for high-yield ethanol production in one ethanologen strain of T. saccharolyticum (M1442), but not another similar strain (M0353).

What causes the different responses to loss of nfnAB in the different strains of T. saccharolyticum engineered for ethanol formation? A possible answer is that one strain uses primarily NADPH for ethanol production, while the other uses primarily NADH.

It is believed that strain M1442 uses the NADPH-linked pathway, based on enzyme assay data from Table 3. It is also believed that a previously-described T. saccharolyticum ethanologen strain, ALK2, also uses this pathway. In strain ALK2, an NADPH preference was seen for ADH, ALDH, and BV reductase activities in cell-free extract (3). Unfortunately genetic manipulation of nfnAB was impossible in ALK2, as ALK2 has marked deletions of ldh and pta, marked with the Kan and Erm resistance markers, the only two markers available for T. saccharolyticum. Both ALK2 and M1442 contain mutations in adhE that have been shown to change the cofactor specificity of AdhE from primarily NADH-linked to NADPH linked (Zheng et al., submitted for publication).

By contrast, the wild-type and LL1145 strain appear to use the NADH-linked ethanol production pathway. Both of these strains use NADH for ADH, ALDH and BV reductase activities in cell-free extracts (Table 13). Furthermore, neither of these strains have mutations in their adhE genes.

TABLE 13
Alcohol/aldehyde/benzyl viologen NAD(P)H activities in cell free extracts

| | Cell free extract | | | | | |
|---|---|---|---|---|---|---|
| | JW/SL- | | | | | |
| | YS485 | LL1144 | M0353 | LL1145 | M1442 | LL1220 |
| | (U/mg protein) | | | | | |
| | | | ΔpyrF | M0353 | Δpta/ack Δldh | M1442 |
| | WT | ΔnfnAB | Δpta/ack Δldh | ΔnfnAB::Kan^{r} | adhE^{G544D} | ΔnfnAB::Kan^{r} |
| NADH-ADH: | 7.06(0.50) | 7.05(1.73) | 0.47(0.11) | 12.40(0.85) | 0.18(0.14) | 0.00(0.02) |
| NADPH-ADH: | 0.95(0.58) | 0.19(0.02) | 0.42(0.05) | 0.00(0.02) | 1.10(0.42) | 0.42(0.10) |
| NADH-ALDH: | 0.41(0.13) | 0.33(0.11) | 1.32(0.31) | 0.21(0.03) | 0.09(0.02) | 0.00(0.16) |
| NADPH-ALDH: | 0.05(0.04) | 0.00(0.12) | 0.08(0.08) | 0.04(0.02) | 0.50(0.05) | 0.41(0.06) |
| NADH-BV: | 0.33(0.05) | 0.33(0.05) | 0.53(0.24) | 0.21(0.04) | 0.20(0.03) | 0.48(0.01) |
| NADPH-BV: | 0.18(0.03) | 0.04(0.03) | 0.48(0.28) | 0.04(0.01) | 0.53(0.23) | 0.06(0.01) |

Average of triplicate experiments,
Standard deviation in parenthesis

Based on enzyme assay data, strain M0353 may be able to use both the NADH and NADPH-linked ethanol production pathways.

Since NADPH is the main cofactor used for ethanol production in the M1442 lineage, without nfnAB, the LL1220 strain may have trouble balancing electron metabolism, in particular NADH/NADPH cofactors and ferredoxin reoxidation. As NFN activity oxidizes NADH and ferredoxin, and reduces NAM⁺, it sits at central junction in electron metabolism. In strain LL1220, loss of ferredoxin oxidation by the NfnAB complex seems to cause significant H₂ formation. NADPH is important for making many biosynthetic components like amino acids, and depletion by NADPH-linked ALDH and ADH would likely affect the growth rates of cells.

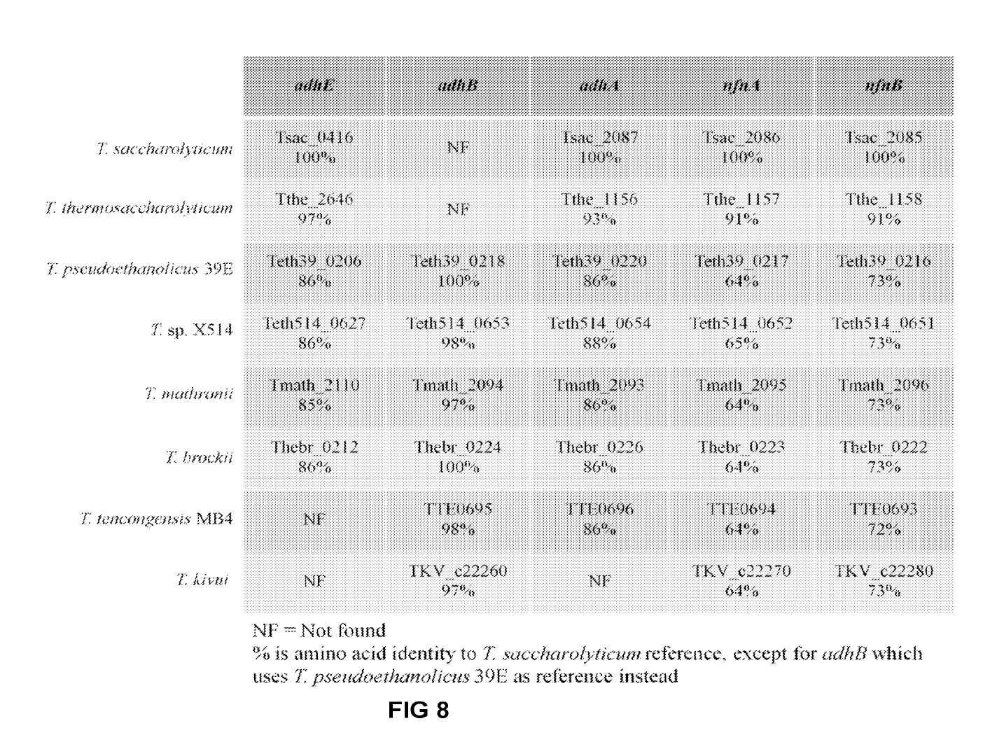

Although mutations in adhE have been shown to give NADPH-linked ADH activity (19, 20), another possible source of NADPH-linked ADH activity is the adhA gene, Tsac_2087. It has been shown that in a T. saccharolyticum adhE deletion strain, there were still significant levels of NADPH-linked ADH activity, suggesting there may be other functional NADPH-linked alcohol dehydrogenases (5). Interestingly, this predicted NADPH-linked alcohol dehydrogenase, adhA, is encoded directly upstream of nfnAB (FIG. 6A). There is evidence that adhA is expressed at very high levels, at least in wild type T. saccharolyticum. A transcriptomic study of this strain found adhA to be consistently among the highest 50 genes expressed (21). The close proximity of these genes suggests a shared biological function and perhaps co-regulation. This gene configuration is shared among Thermoanaerobacter and Thermoanaerobacterium species previously investigated for ethanol formation like Thermoanaerobacter mathranii, Thermoanaerbacter pseudoethanolicus, Thermoanaerobacter brockii (FIG. 8). Many of these bacteria have demonstrated high ethanol yields (13, 22, 23). Interestingly, many Thermoanaerobacter species also encode a predicted adhB nearby as well, which is a Zn-dependent bifunctional alcohol/aldehyde dehydrogenase that primarily uses NADPH as a cofactor instead of NADH (24-26).

There is previous evidence for NADPH as the primary cofactor utilized for ethanol formation. In a T. pseudoethanolicus strain engineered for ethanol tolerance, it was noted that NADH-linked ADH, ALDH, and FNOR activities were lost, while NADPH-linked activities remained (27). While this strain produced less ethanol than the parent strain, the ethanol tolerant strain still produced high yields of ethanol (28). Additionally, it was shown that T. brockii had mostly NADPH-linked ADH activity (13). A meta-analysis of metabolic pathways in select fermentative microorganisms noted that all major ethanol formers included adhE except one (29). The exception was Thermoanaerobacter tengcongensis sp. MB4, which authors noted only encoded alcohol dehydrogenases and lacked aldehyde dehydrogenases, yet was reported to produce significant amounts of ethanol. This locus may explain ethanol formation in T. tengcongensis sp. MB4. One of alcohol dehydrogenase genes, TTE0695, encodes a predicted adhB, which shares high similarity (>95% identity) to the adhB from T. mathranii and T. pseudoethanolicus. AdhB can catalyze a NADPH-dependant conversion of acetyl-CoA to ethanol (reaction 3) (24) and could be the source of ethanol formation in T. tengcongensis sp. MB4. Both the ALDH and ADH reactions in T. tengcongensis sp. MB4 were shown to be NADPH-linked (30) and could possibly be catalyzed by AdhB and/or AdhA.

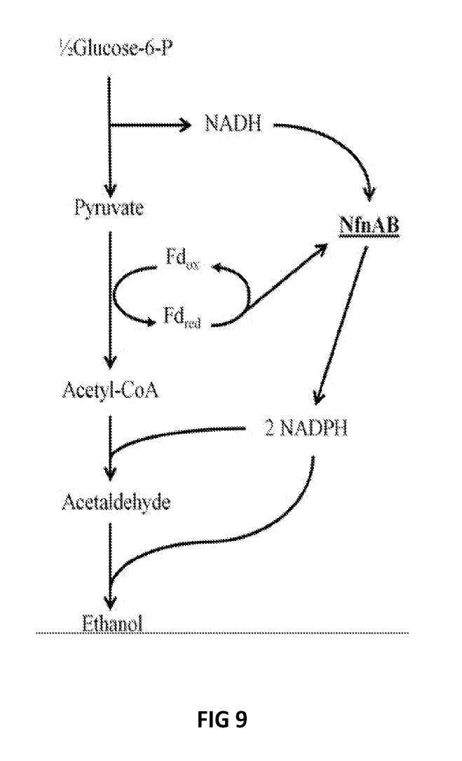

How does T. saccharolyticum make high yields of ethanol? If pyruvate:ferredoxin oxidoreductase (PFOR) is the enzyme responsible for pyruvate oxidation as believed (4), then there must be electron transfer from reduced ferredoxin to NAD(P)⁺. It has been shown that the NfnAB complex can play a role in that electron transfer. However, it appears there may be other unidentified FNOR-like enzymes responsible for transfer of electrons from reduced ferredoxin to NAD⁺. NfnAB does not strongly reduce benzyl viologen with NADH (6), yet substantial NADH:BV reductase activities were previously seen in cell-free extracts (3, 4). Indeed, in cell-free extracts lacking nfnAB high levels of NADH:BV reductase activity were observed, suggesting that there may be other enzymes with FNOR activity. Thus, two different mechanisms for stoichiometric yield of ethanol in T. saccharolyticum have been proposed, one based on NADPH and NfnAB, the other based on NADH and a yet undescribed NADH-FNOR (FIG. 9). Much is still unknown about the enzymes involved in the metabolism of anaerobic thermophiles, especially in the context of their importance in end product formation.

The presence of two other NADPH-generating reactions was also demonstrated: glucose-6-phosphate dehydrogenase and NADP⁺-linked isocitrate dehydrogenase, which are possibly indicative of an oxidative pentose phosphate pathway and a tricarboxylic acid cycle, respectively. A bifurcated TCA cycle has been previously demonstrated in Clostridium acetobutylicum as having a role in biosynthetic reactions (31), and this feature may be present in T. saccharolyticum as well. These reactions may be responsible for the limited ethanol production observed when nfnAB was deleted from strain M1442, although additional genetic work will be necessary for definitive confirmation.

In conclusion, it is disclosed here the first deletion of nfnAB and characterize nfnAB's role in T. saccharolyticum metabolism. A new native gel based assay was described for detection of NfnAB. Biochemical and fermentation product changes resulting from the genetic manipulation of nfnAB were also described which showed that nfnAB is important for ethanol formation at high yield in strain M1442. Additionally, these results suggested a potential role of adhA in T. saccharolyticum ethanol formation. While NfnAB can be important for ethanol formation, it is not always essential, and provide evidence of a different NADH (instead of NADPH)-linked ethanol production pathway in strain M0353, which involves an NADH-linked FNOR (which has not yet been linked to a specific gene). Finally, it was shows that glucose-6-phosphate dehydrogenase and isocitrate dehydrogenase are other potential sources of NADPH generation. Although T. saccharolyticum was successfully engineered for high ethanol formation by inactivating acetate and lactate production, the identity, function, and interaction of enzymes involved in ethanol formation are poorly understood. NfnAB is believed to be distributed among a wide variety of microbes with diverse energy metabolisms, but its function and importance in these microbes remains largely unknown. Elucidating these pathways is an important part in understanding the metabolism and physiology of anaerobic microorganisms.

### Example 4 Expression of the 3-Gene T. saccharolyticum Pyruvate to Ethanol Pathway in C. thermocellum Increases Ethanol Yield

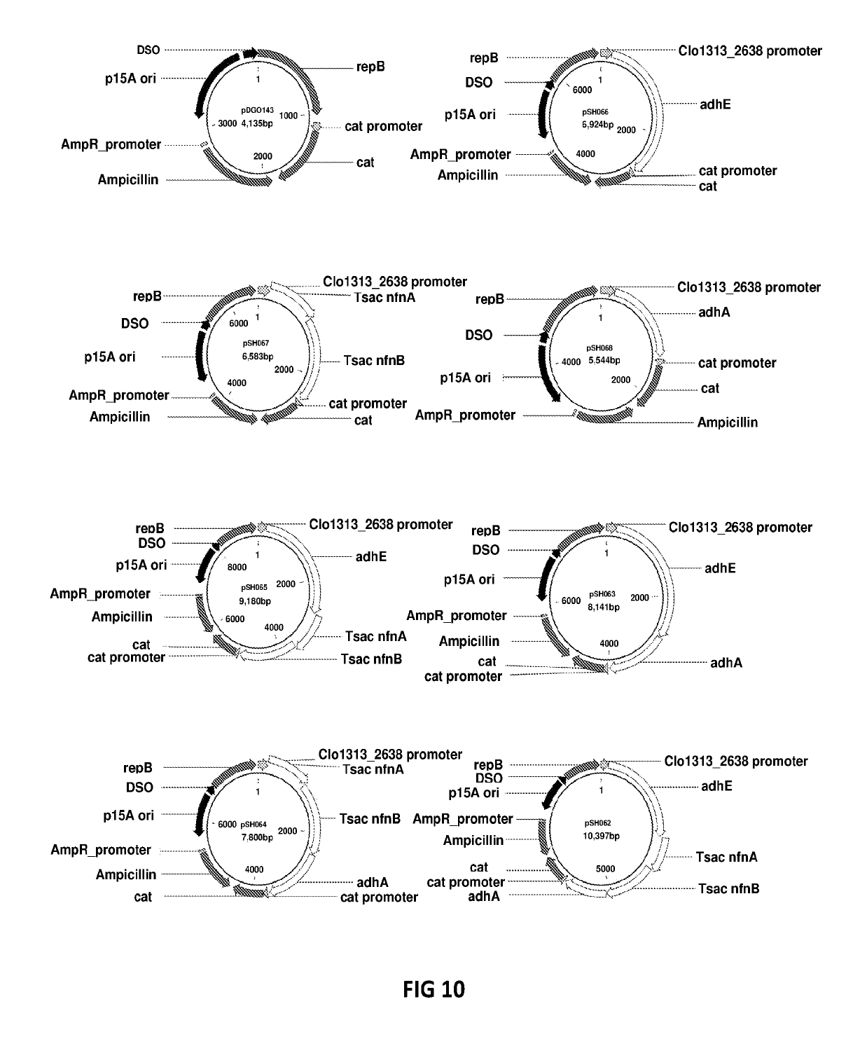

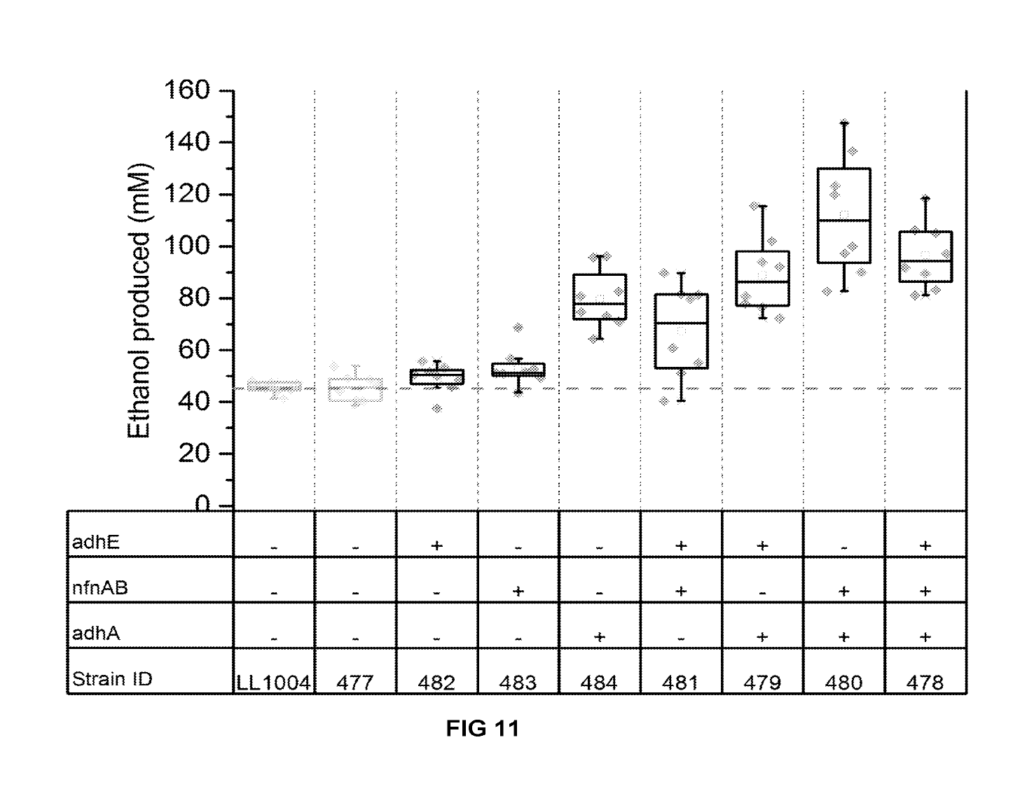

In this Example, the T. saccharolyticum pathway (adhA, nfnA, nfnB and/or adhEG544D) was expressed from a plasmid (FIG. 10). Plasmid construction was based on standard molecular biology techniques. Plasmid transformation into C. thermocellum had been described previously. Olson, D. G. & Lynd, L. R. in Methods Enzymol. (Gilbert, H. J.) Volume 510, 317-330 (Academic Press, 2012). Plasmid pDGO143 was the empty vector control. All other plasmids were based on pDGO143. The construction of plasmid pDGO143 had been previously described. Hon, S. et al. Development of a Plasmid-Based Expression System in Clostridium thermocellum and its use to Screen Heterologous Expression of bifunctional alcohol dehydrogenases (adhEs). Metab. Eng. Commun. 120-129 (2016). Plasmids pSH062 through pSH068 included various combinations of the T. saccharolyticum pathway genes expressed by the C. thermocellum Clo1313_2638 promoter. The result of plasmid-based expression is shown in FIG. 11. Strains 482, 483 and 484 show the individual contribution of adhEG544D, nfnAB and adhA, respectively. Strains 481, 479 and 480 show the effect of combinations of two or three genes (note that although nfnA and nfnB are always expressed together, this is not strictly necessary). Strain 478 shows the presence of all four genes. Although strain 480 (which does not have adhEG544D) worked best in this example, there were some cases where the presence of adhEG544D does improve ethanol production (compare strains 483 and 481). In strain 480, the ethanol titer was improved by 2.6-fold, compared to the negative control (strain 477).

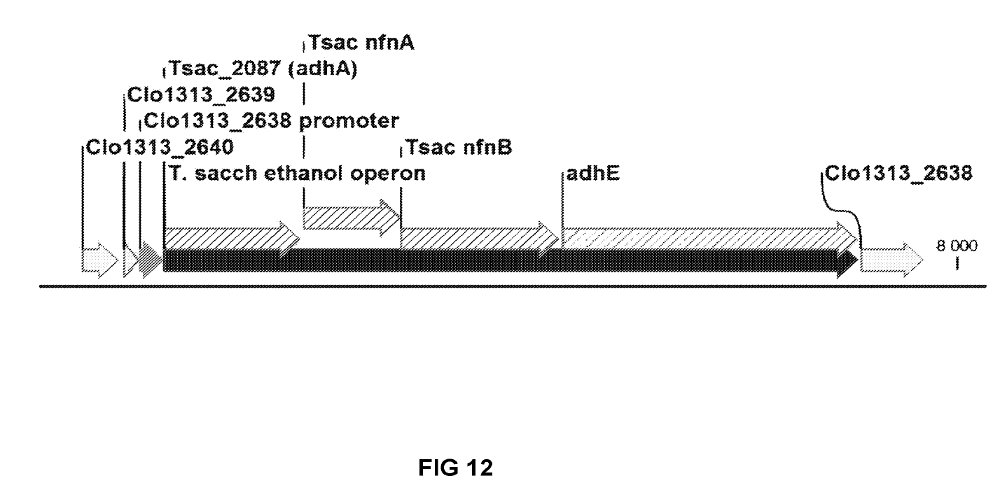

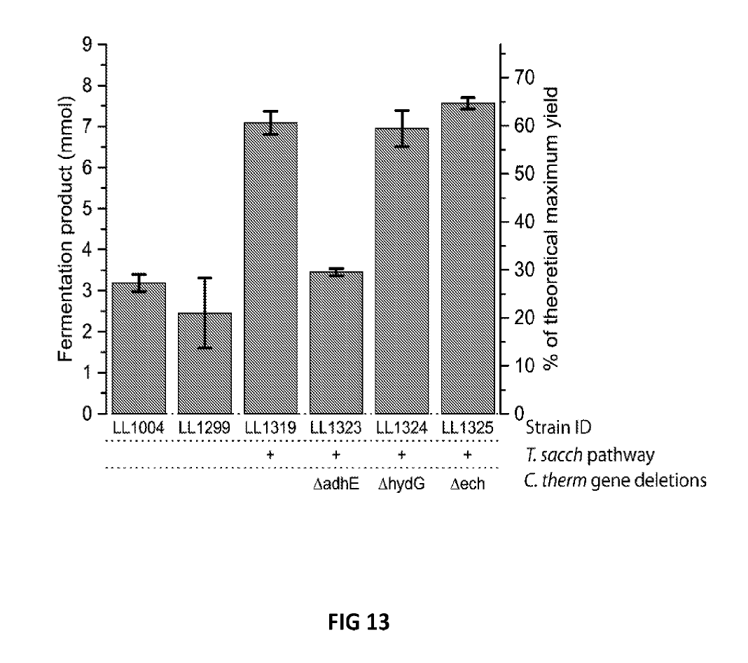

In another test, the T. saccharolyticum pathway was expressed from the C. thermocellum chromosome. The insertion of DNA into the C. thermocellum chromosome had been described previously. FIG. 12 shows the arrangement of the genetic locus. The effect of the pathway is shown in FIG. 13. Strain LL1004 is wild type C. thermocellum. Strain LL1299 had an additional deletion of Clo1313_0478 to allow improved transformation efficiency. This deletion had no significant effect on ethanol production (compare LL1004 with LL1299). In the presence of the pathway, ethanol titer is improved by 2.8-fold (compare strains LL1319 with LL1299). When the native C. thermocellum adhE was deleted (strain LL1323), ethanol production decreased, but did not decrease to zero. This demonstrates that the T. saccharolyticum adhEG544D was functional. It also shows that the native C. thermocellum adhE was playing a role in ethanol production, even when the T. saccharolyticum pathway was present.

The contents of all cited references (including literature references, patents, patent applications, and websites) that may be cited throughout this application or listed below are hereby expressly incorporated by reference in their entirety for any purpose into the present disclosure. The disclosure may employ, unless otherwise indicated, conventional techniques of microbiology, molecular biology and cell biology, which are well known in the art.

The disclosed methods and systems may be modified without departing from the scope hereof. It should be noted that the matter contained in the above description or shown in the accompanying drawings should be interpreted as illustrative and not in a limiting sense.

### LIST OF REFERENCES

The following references, patents and publication of patent applications are either cited in this disclosure or are of relevance to the present disclosure. All documents listed below, along with other papers, patents and publication of patent applications cited throughout this disclosures, are hereby incorporated by reference as if the full contents are reproduced herein.
Lynd L R, Weimer P J, van Zyl W H, Pretorius I S. 2002. Microbial cellulose utilization: fundamentals and biotechnology. Microbiol. Mol. Biol. Rev. 66:506-577.
Blumer-Schuette S E, Brown S D, Sander K B, Bayer E A, Kataeva I, Zurawski J V, Conway J M, Adams M W W, Kelly R M. 2014. Thermophilic lignocellulose deconstruction. FEMS Microbiol. Rev. 38:393-448.
Mai V, Lorenz W W, Wiegel J. 1997. Transformation of Thermoanaerobacterium sp. strain JW/SL-YS485 with plasmid pIKM1 conferring kanamycin resistance. FEMS Microbiol. Lett. 148:163-167.
Shaw A J, Covalla S F, Miller B B, Firliet B T, Hogsett D A, Herring C D. 2012. Urease expression in a Thermoanaerobacterium saccharolyticum ethanologen allows high titer ethanol production. Metab. Eng. 14:528-532.
Shao X J, Raman B, Zhu M J, Mielenz J R, Brown S D, Guss A M, Lynd L R. 2011. Mutant selection and phenotypic and genetic characterization of ethanol-tolerant strains of Clostridium thermocellum. Appl. Microbiol. Biotechnol. 92:641-652.
Williams T I, Combs J C, Lynn B C, Strobel H J. 2007. Proteomic profile changes in membranes of ethanol-tolerant Clostridium thermocellum. Appl. Microbiol. Biotechnol. 74:422-432.
Sudha Rani K, Swamy M, Sunitha D, Haritha D, Seenayya G. 1996. Improved ethanol tolerance and production in strains of Clostridium thermocellum. World J. Microbiol. Biotechnol. 12:57-60.
Sato K, Tomita M, Yonermura S, Goto S, Sekine K, Okuma E, Takagi Y, Hon-nami K, Saiki T. 1993. Characterization of and ethanol hyper-production by Clostridium thermocellum I-1-B. Biosci. Biotechnol. Biochem. 57:2116-2121.
Argyros D A, Tripathi S A, Barrett T F, Rogers S R, Feinberg L F, Olson D G, Foden J M, Miller B B, Lynd L R, Hogsett D A, Caiazza N C. 2011. High ethanol titers from cellulose by using metabolically engineered thermophilic, anaerobic microbes. Appl. Environ. Microbiol. 77:8288-8294.
Shaw A J, Podkaminer K K, Desai S G, Bardsley J S, Rogers S R, Thorne P G, Hogsett D A, Lynd L R. 2008. Metabolic engineering of a thermophilic bacterium to produce ethanol at high yield. Proc. Natl. Acad. Sci. U.S.A 105:13769-13774.
König S. 1998. Subunit structure, function and organisation of pyruvate decarboxylases from various organisms. Biochim Biophys. Acta 1385:271-286.
Extance J, Crennell S J, Eley K, Cripps R, Hough D W, Danson M J. 2013. Structure of a bifunctional alcohol dehydrogenase involved in bioethanol generation in Geobacillus thermoglucosidasius. Acta Crystallogr. Sect. D Biol. Crystallogr. 69:2104-2115.
Membrillo-Herna J, Echave P, Cabiscol E, Tamarit J, Ros J, Lin E C C. 2000. Evolution of the adhE gene product of Escherichia coli from a functional reductase to a dehydrogenase. J. Biol. Chem. 275:33869-33875.
Yao S. 2008. Ph.D. thesis. Technical University of Denmark, Denmark.
Peng H, Wu G, Shao W. 2008. The aldehyde/alcohol dehydrogenase (AdhE) in relation to the ethanol formation in Thermoanaerobacter ethanolicus JW200. Anaerobe 14:125-127.
Bhandiwad A, Shaw A J, Guss A, Guseva A, Bahl H, Lynd L R. 2014. Metabolic engineering of Thermoanaerobacterium saccharolyticum for n-butanol production. Metab. Eng. 21:17-25.
Pineda E, Encalada R, Olivos-García A, Néquiz M, Moreno-Sánchez R, Saavedra E. 2013. The bifunctional aldehyde-alcohol dehydrogenase controls ethanol and acetate production in Entamoeba histolytica under aerobic conditions. FEBS Lett. 587:178-184.
Boxma B, Voncken F, Jannink S, van Alen T, Akhmanova A, van Weelden S W H, van Hellemond J J, Ricard G, Huynen M, Tielens A G M, Hackstein J H P. 2004. The anaerobic chytridiomycete fungus Piromyces sp. E2 produces ethanol via pyruvate:formate lyase and an alcohol dehydrogenase E. Mol. Microbiol. 51:1389-1399.
Atteia A, van Lis R, Mendoza-Hernandez G, Henze K, Martin W, Riveros-Rosas H, Gonzalez-Halphen D. 2003. Bifunctional aldehyde/alcohol dehydrogenase (ADHE) in chlorophyte algal mitochondria. Plant Mol. Biol. 53:175-188.
Lo J, Zheng T, Hon S, Olson D G, Lynd L R. 2015. The bifunctional alcohol and aldehyde dehydrogenase gene, adhE, is necessary for ethanol production in Clostridium thermocellum and Thermoanaerobacterium saccharolyticum. J. Bacteriol. JB02450-14.
Brown S D, Guss A M, Karpinets T V, Parks J M, Smolin N, Yang S, Land M L, Klingeman D M, Bhandiwad A, Rodriguez Jr. M, Raman B, Shao X, Mielenz J R, Smith J C, Keller M, Lynd L R. 2011. Mutant alcohol dehydrogenase leads to improved ethanol tolerance in Clostridium thermocellum. Proc. Natl. Acad. Sci. 108:13752-13757.
Olson D G, Lynd L R. 2012. Transformation of Clostridium thermocellum by electroporation. Methods in Enzymology, 1st ed. Elsevier Inc.
Shaw A J, Hogsett D A, Lynd L R. 2010. Natural competence in Thermoanaerobacter and Thermoanaerobacterium species. Appl. Environ. Microbiol. 76:4713-4719.
Zhou J, Olson D G, Argyros D A, Deng Y, van Gulik W M, van Dijken J P, Lynd L R. 2013. Atypical glycolysis in Clostridium thermocellum. Appl. Environ. Microbiol. 79:3000-3008.
Zhang Y, Lynd L R. 2003. Quantification of cell and cellulase mass concentrations during anaerobic cellulose fermentation: development of an enzyme-linked immunosorbent assay-based method with application to Clostridium thermocellum batch cultures. Anal. Chem. 75:3131-3139.
Espinosa A, Yan L, Zhang Z, Foster L, Clark D, Li E, Stanley Jr. SL. 2001. The bifunctional Entamoeba histolytica alcohol dehydrogenase 2 (EhADH2) protein is necessary for amebic growth and survival and requires an intact C-terminal domain for both alcohol dehydrogenase and acetaldehyde dehydrogenase activity. J. Biol. Chem. 276:20136-20143.
Schwarzenbacher R, von Delft F, Canaves J M, Brinen L S, Dai X, Deacon A M, Elsliger M A, Eshaghi S, Floyd R, Godzik A, Grittini C, Grzechnik S K, Guda C, Jaroszewski L, Karlak C, Klock H E, Koesema E, Kovarik J S, Kreusch A, Kuhn P, Lesley S A, Mcmullan D, Mcphillips T M, Miller M A, Miller M D, Morse A, Moy K, Ouyang J, Page R, Robb A, Rodrigues K, Selby T L, Spraggon G, Stevens R C, van den Bedem H, Velasquez J, Vincent J, Wang X, West B, Wolf G, Hodgson K O, Wooley J, Wilson I A. 2004. Crystal structure of an iron-containing 1,3-propanediol dehydrogenase (TM0920) from Thermotoga maritima at 1.3 Å resolution. PROTEINS Struct. Funct. Bioinforma. 54:174-177.
Jorgensen W L, Chandrasekhar J, Madura J D, Impey R W, Klein M L. 1983. Comparison of simple potential functions for simulating liquid water. J. Chem. Phys. 79:926-935.
Jo S, Kim T, Iyer V G, Im W. 2008. Software news and updates CHARMM-GUI: A web-based graphical user interface for CHARMM. J. Comput. Chem. 29:1859-1865.
Koukos P I, Glykos N M. 2013. Grcarma: A fully automated task-oriented interface for the analysis of molecular dynamics trajectories. J. Comput. Chem. 34:2310-2312.
Yao S, Mikkelsen M J. 2010. Identification and overexpression of a bifunctional aldehyde/alcohol dehydrogenase responsible for ethanol production in Thermoanaerobacter mathranii. J. Mol. Microbiol. Biotechnol. 19:123-133.
Dailly Y, Bunch P, Clark D. 2000. Comparison of the fermentative alcohol dehydrogenases of Salmonella typhimurium and Escherichia coli. Microbios 103:179-196.
Pei J, Zhou Q, Jiang Y, Le Y, Li H, Shao W, Wiegel J. 2010. Thermoanaerobacter spp. control ethanol pathway via transcriptional regulation and versatility of key enzymes. Metab. Eng. 12:420-428.
Arnau J, Jorgensen F, Madsen S M, Vrang A, Israelsen H. 1998. Cloning of the Lactococcus lactis adhE gene, encoding a multifunctional alcohol dehydrogenase, by complementation of a fermentative mutant of Escherichia coli. J. Bacteriol. 180:3049-3055.
Bruchhaus I, Tannich E. 1994. Purification and molecular characterization of the NAD(+)-dependent acetaldehyde/alcohol dehydrogenase from Entamoeba histolytica. Biochem. J. 303:743-748.
Koo O K, Jeong D W, Lee J M, Kim M J, Lee J H, Chang H C, Kim J H, Lee H J. 2005. Cloning and characterization of the bifunctional alcohol/acetaldehyde dehydrogenase gene (adhE) in Leuconostoc mesenteroides isolated from kimchi. Biotechnol. Lett. 27:505-510.
Chen M, Li E, Stanley S L. 2004. Structural analysis of the acetaldehyde dehydrogenase activity of Entamoeba histolytica alcohol dehydrogenase 2 (EhADH2), a member of the ADHE enzyme family Mol. Biochem. Parasitol. 137:201-205.
Montella C, Bellsolell L, Perez-Luque R, Badia J, Baldoma L, Coll M, Aguilar J. 2005. Crystal structure of an iron-dependent group III dehydrogenase that interconverts L-lactaldehyde and L-1,2-propanediol in Escherichia coli. J. Bacteriol. 187:4957-4966.
Huang H, Wang S, Moll J, Thauer R K. 2012. Electron bifurcation involved in the energy metabolism of the acetogenic bacterium Moorella thermoacetica growing on glucose or H2 plus CO2. J. Bacteriol. 194:3689-3699.
Currie D H, Guss A M, Herring C D, Giannone R J, Johnson C M, Lankford P K, Brown S D, Hettich R L, Lynd L R. 2014. Profile of secreted hydrolases, associated proteins, and SlpA in Thermoanaerobacterium saccharolyticum during the degradation of hemicellulose. Appl. Environ. Microbiol. 80:5001-5011.
Verbeke T J, Zhang X, Henrissat B, Spicer V, Rydzak T, Krokhin O V., Fristensky B, Levin D B, Sparling R. 2013. Genomic evaluation of Thermoanaerobacter spp. for the construction of designer co-cultures to improve lignocellulosic biofuel production. PLoS One 8:1-18.
Radianingtyas H, Wright P C. 2003. Alcohol dehydrogenases from thermophilic and hyperthermophilic archaea and bacteria. FEMS Microbiol. Rev. 27:593-616.
Burdette D, Zeikus J G. 1994. Purification of acetaldehyde dehydrogenase and alcohol dehydrogenases from Thermoanaerobacter ethanolicus 39E and characterization of the secondary-alcohol dehydrogenase (20 Adh) as a bifunctional alcohol dehydrogenase-acetyl-CoA reductive thioesterase. Biochem. J. 302:163-170.
Lamed R, Zeikus J G. 1980 Ethanol production by thermophilic bacteria: relationship between fermentation product yields of and catabolic enzyme activities in Clostridium thermocellum and Thermoanaerobium brockii. J. Bacteriol. 144:569-578.
Neale A D, Scopes R K, Kelly J M, Wettenhall R E H. 1986. The two alcohol dehydrogenases of Zymomonas mobilis purification by differential dye ligand chromatography, molecular characterisation and physiological roles. Eur. J. Biochem. 154:119-124.
Chen Z, Lee W R, Chang S H. 1991. Role of aspartic acid 38 in the cofactor specificity of Drosophila alcohol dehydrogenase. Eur. J. Biochem. 202:263-267.
Kessler D, Herth W, Knappe J. 1992. Ultrastructure and pyruvate formate-lyase radical quenching property of the multienzymic AdhE protein of Escherichia coli. J. Biol. Chem. 267:18073-18073.
Biswas R, Zheng T, Olson D G, Lynd L R, Guss A M. 2015. Elimination of hydrogenase active site assembly blocks H2 production and increases ethanol yield in Clostridium thermocellum. Biotechnol. Biofuels. In press.
Wyman C E. 2007. What is (and is not) vital to advancing cellulosic ethanol. Trends Biotechnol. 25:153-157.
Olson D G, McBride J E, Shaw A J, Lynd L R, Joe Shaw A. 2012. Recent progress in consolidated bioprocessing. Curr. Opin. Biotechnol. 23:396-405.
Shaw A J, Podkaminer K K, Desai S G, Bardsley J S, Rogers S R, Thorne P G, Hogsett D A, Lynd L R. 2008. Metabolic engineering of a thermophilic bacterium to produce ethanol at high yield. Proc. Natl. Acad. Sci. U.S.A 105:13769-13774.
Shaw J A, Jenney F E, Adams M W W, Lynd L R. 2008. End-product pathways in the xylose fermenting bacterium, Thermoanaerobacterium saccharolyticum. Enzyme Microb. Technol. 42:453-458.
Lo J, Zheng T, Hon S, Olson D G, Lynd L R. 2015. The bifunctional alcohol and aldehyde dehydrogenase gene, adhE, is necessary for ethanol production in Clostridium thermocellum and Thermoanaerobacterium saccharolyticum. J. Bacteriol. 197:1386-1393.
Wang S, Huang H, Moll J, Thauer R K. 2010. NADP+ reduction with reduced ferredoxin and NADP+ reduction with NADH are coupled via an electron-bifurcating enzyme complex in Clostridium kluyveri. J. Bacteriol. 192:5115-23.
Buckel W, Thauer R K. 2013. Energy conservation via electron bifurcating ferredoxin reduction and proton/Na(+) translocating ferredoxin oxidation. Biochim Biophys. Acta 1827:94-113.
Tripathi S A, Olson D G, Argyros D A, Miller B B, Barrett T F, Murphy D M, McCool J D, Warner A K, Rajgarhia V B, Lynd L R, Hogsett D A, Caiazza N C. 2010. Development of pyrF-based genetic system for targeted gene deletion in Clostridium thermocellum and creation of a pta mutant. Appl. Environ. Microbiol. 76:6591-6599.
Shanks R M Q, Caiazza N C, Hinsa S M, Toutain C M, O'Toole G A. 2006. Saccharomyces cerevisiae-based molecular tool kit for manipulation of genes from gram-negative bacteria. Appl. Environ. Microbiol. 72:5027-5036.
Shaw a J, Hogsett D a, Lynd L R. 2009. Identification of the [FeFe]-hydrogenase responsible for hydrogen generation in Thermoanaerobacterium saccharolyticum and demonstration of increased ethanol yield via hydrogenase knockout. J. Bacteriol. 191:6457-64.
Zhou J, Olson D G, Argyros D A, Deng Y, van Gulik W M, van Dijken J P, Lynd L R. 2013. Atypical glycolysis in Clostridium thermocellum. Appl. Environ. Microbiol. 79:3000-8.
Fournier M, Dermoun Z, Durand M-C, Dolla A. 2004. A new function of the Desulfovibrio vulgaris Hildenborough [Fe] hydrogenase in the protection against oxidative stress. J. Biol. Chem. 279:1787-93.
Lamed R, Zeikus J G. 1980 Ethanol production by thermophilic bacteria: relationship between fermentation product yields of and catabolic enzyme activities in Clostridium thermocellum and Thermoanaerobium brockii. J. Bacteriol. 144:569-78.
Thorsness P E, Koshland D E. 1987. Inactivation of isocitrate dehydrogenase by phosphorylation is mediated by the negative charge of the phosphate. J. Biol. Chem. 262:10422-10425.
Van der Veen D, Lo J, Brown S D, Johnson C M, Tschaplinski T J, Martin M, Engle N L, van den Berg R a, Argyros A D, Caiazza N C, Guss A M, Lynd L R. 2013. Characterization of Clostridium thermocellum strains with disrupted fermentation end-product pathways. J. Ind. Microbiol. Biotechnol. 40:725-34.
Shaw a J, Covalla S F, Hogsett D a, Herring C D. 2011. Marker removal system for Thermoanaerobacterium saccharolyticum and development of a markerless ethanologen. Appl. Environ. Microbiol. 77:2534-6.
Lee J M, Venditti R A, Jameel H, Kenealy W R. 2011. Detoxification of woody hydrolyzates with activated carbon for bioconversion to ethanol by the thermophilic anaerobic bacterium Thermoanaerobacterium saccharolyticum. Biomass and Bioenergy 35:626-636.
Currie D H, Herring C D, Guss A M, Olson D G, Hogsett D a, Lynd L R. 2013. Functional heterologous expression of an engineered full length CipA from Clostridium thermocellum in Thermoanaerobacterium saccharolyticum. Biotechnol. Biofuels 6:32.
Biswas R, Zheng T, Olson D G, Lynd L R, Guss A M. 2015. Elimination of hydrogenase active site assembly blocks H2 production and increases ethanol yield in Clostridium thermocellum. Biotechnol. Biofuels 8:20.
Brown S D, Guss A M, Karpinets T V, Parks J M, Smolin N, Yang S, Land M L, Klingeman D M, Bhandiwad A, Rodriguez M, Raman B, Shao X, Mielenz J R, Smith J C, Keller M, Lynd L R. 2011. Mutant alcohol dehydrogenase leads to improved ethanol tolerance in Clostridium thermocellum. Proc. Natl. Acad. Sci. U.S.A. 108:13752-7.
Currie D H, Guss A M, Herring C D, Giannone R J, Johnson C M, Lankford P K, Brown S D, Hettich R L, Lynd L R. 2014. Profile of secreted hydrolases, associated proteins, and SlpA in Thermoanaerobacterium saccharolyticum during the degradation of hemicellulose. Appl. Environ. Microbiol. 80:5001-11.
Yao S, Mikkelsen M J. 2010. Metabolic engineering to improve ethanol production in Thermoanaerobacter mathranii. Appl. Microbiol. Biotechnol. 88:199-208.
Wiegel J, Ljungdahl L G. 1981. Thermoanaerobacter ethanolicus gen. nov., spec. nov., a new, extreme thermophilic, anaerobic bacterium. Arch. Microbiol. 128:343-348.
Burdette D, Zeikus J G. 1994. Purification of acetaldehyde dehydrogenase and alcohol dehydrogenases from Thermoanaerobacter ethanolicus 39E and characterization of the secondary-alcohol dehydrogenase (2 degrees Adh) as a bifunctional alcohol dehydrogenase-acetyl-CoA reductive thioest. Biochem. J. 302:163-70.
Yao S, Mikkelsen M J. 2010. Identification and overexpression of a bifunctional aldehyde/alcohol dehydrogenase responsible for ethanol production in Thermoanaerobacter mathranii. J. Mol. Microbiol. Biotechnol. 19:123-133.
Bryant F O, Wiegel J, Ljungdahl L G. 1988. Purification and Properties of Primary and Secondary Alcohol Dehydrogenases from Thermoanaerobacter ethanolicus. Appl. Environ. Microbiol. 54:460-465.
Lovitt R W, Shen G J, Zeikus J G. 1988. Ethanol production by thermophilic bacteria: biochemical basis for ethanol and hydrogen tolerance in Clostridium thermohydrosulfuricum. J. Bacteriol. 170:2809-15.
Lovitt R W, Longin R, Zeikus J G. 1984 Ethanol Production by Thermophilic Bacteria: Physiological Comparison of Solvent Effects on Parent and Alcohol-Tolerant Strains of Clostridium thermohydrosulfuricum. Appl. Envir. Microbiol. 48:171-177.
Carere C R, Rydzak T, Verbeke T J, Cicek N, Levin D B, Sparling R. 2012. Linking genome content to biofuel production yields: a meta-analysis of major catabolic pathways among select H2 and ethanol-producing bacteria. BMC Microbiol. 12:295.
Soboh B, Linder D, Hedderich R. 2004. A multisubunit membrane-bound lNiFel hydrogenase and an NADH-dependent Fe-only hydrogenase in the fermenting bacterium Thermoanaerobacter tengcongensis. Microbiology 150:2451-63.
Amador-Noguez D, Feng X-J, Fan J, Roquet N, Rabitz H, Rabinowitz J D. 2010. Systems-level metabolic flux profiling elucidates a complete, bifurcated tricarboxylic acid cycle in Clostridium acetobutylicum. J. Bacteriol. 192:4452-61.
Lee Y-E, Jain M K, Lee C, Zeikus J G: Taxonomic Distinction of Saccharolytic Thermophilic Anaerobes: Description of Thermoanaerobacterium xylanolyticum gen. nov., sp. nov., and Thermoanaerobacterium saccharolyticum gen. nov., sp. nov.; Reclassification of Thermoanaerobium brockii, Clostridium. Int J Syst Bacteriol 1993, 43:41-51.
Shaw A J, Podkaminer K, Desai S, Bardsley J, Rogers S, Thorne P, Hogsett D A, Lynd L R: Metabolic engineering of a thermophilic bacterium to produce ethanol at high yield. Proc Natl Acad Sci USA 2008, 105:13769-13774.
Shaw A J, Covalla S F, Miller B B, Firliet B T, Hogsett D A, Herring C D: Urease expression in a Thermoanaerobacterium saccharolyticum ethanologen allows high titer ethanol production. Metab Eng 2012, 14:528-32.
Herring C D, Kenealy W R, Shaw A J, Raman B, Tschaplinski T J, Brown S D, Davison B H, Covalla S F, Sillers W R, Xu H, Tsakraklides V, Hogsett D A: Final Report on Development of Thermoanaerobacterium Saccharolyticum for the Conversion of Lignocellulose to Ethanol. Golden, Colo. (United States); 2012.
Lynd L R, Weimer P J, van Zyl W H, Pretorius I S: Microbial cellulose utilization: fundamentals and biotechnology. Microbiol Mol Biol Rev 2002, 66:506-577.
Lynd L R, van Zyl W H, McBride J E, Laser M: Consolidated bioprocessing of cellulosic biomass: an update. Curr Opin Biotechnol 2005, 16:577-583.
Shaw A J, Jenney Jr F E, Adams M W W, Lynd L R: End-product pathways in the xylose fermenting bacterium, Thermoanaerobacterium saccharolyticum. Enzyme Microb Techno12008, 42:453-458.
Kanehisa M, Goto S: KEGG: Kyoto Encyclopedia of Genes and Genomes. 2000, 28:27-30.
Kanehisa M, Goto S, Sato Y, Kawashima M, Furumichi M, Tanabe M: Data, information, knowledge and principle: back to metabolism in KEGG. Nucleic Acids Res 2014, 42 (Database issue):D199-205.
Chabriere E, Cavazza C, Contreras-Martel C, Fontecilla-Camps J C: Pyruvate-ferredoxin oxidoreductase. In Encyclopedia of Inorganic and Bioinorganic Chemistry; 2011:1-13.
Zhou J, Olson D G, Argyros D, Deng Y, van Gulik W, van Dijken J, Lynd L R: Atypical glycolysis in Clostridium thermocellum. Appl Environ Microbiol 2013, 79:3000-3008.
Hogsett D A: Cellulose hydrolysis and fermentation by Clostridium thermocellum for the production of ethanol. Dartmouth College, Hanover, N.H.; 1995.
Olson D G, Lynd L R: Computational design and characterization of a temperature-sensitive plasmid replicon for gram positive thermophiles. J Biol Eng 2012, 6:5.
Bertani G: Studies on lysogenesis. I. The mode of phage liberation by lysogenic Escherichia coli. J Bacteriol 1951, 62:293-300.
Desai S G, Guerinot M L, Lynd L R: Cloning of L-lactate dehydrogenase and elimination of lactic acid production via gene knockout in Thermoanaerobacterium saccharolyticum JW/SL-YS485. Appl Microbiol Biotechnol 2004, 65:600-5.
Shaw A J, Hogsett D A, Lynd L R: Natural competence in Thermoanaerobacter and Thermoanaerobacterium species. Appl Environ Microbiol 2010, 76:4713-9.
Shaw A J, Hogsett D A, Lynd L R: Identification of the [FeFe]-hydrogenase responsible for hydrogen generation in Thermoanaerobacterium saccharolyticum and demonstration of increased ethanol yield via hydrogenase knockout. J Bacteriol 2009, 191:6457-64.
Kruger N: The Bradford method for protein quantitation. Methods Mol Biol 1994, 32:9-15.
Ma K, Adams M W: Ferredoxin:NADP oxidoreductase from Pyrococcus furiosus. In Methods in Enzymology. Volume 334. Elsevier; 2001:40-45. [Methods in Enzymology]
Altschul S F, Gish W, Miller W, Myers E W, Lipman D J: Basic local alignment search tool. J Mol Biol 1990, 215:403-10.
Zhang Y P, Lynd L R: Regulation of cellulase synthesis in batch and continuous cultures of Clostridium thermocellum. J Bacteriol 2005, 187:99-106.
Van der Veen D, Lo J, Brown S D, Johnson C M, Tschaplinski T J, Martin M, Engle N L, van den Berg R A, Argyros A D, Caiazza N C, Guss A M, Lynd L R: Characterization of Clostridium thermocellum strains with disrupted fermentation end-product pathways. J Ind Microbiol Biotechnol 2013, 40:725-34.
Currie D H, Guss A M, Herring C D, Giannone R J, Johnson C M, Lankford P K, Brown S D, Hettich R L, Lynd L R: Profile of secreted hydrolases, associated proteins, and SlpA in Thermoanaerobacterium saccharolyticum during the degradation of hemicellulose. Appl Environ Microbiol 2014, 80:5001-11.
Ma K, Hutchins A, Sung S J, Adams M W: Pyruvate ferredoxin oxidoreductase from the hyperthermophilic archaeon, Pyrococcus furiosus, functions as a CoA-dependent pyruvate decarboxylase. Proc Natl Acad Sci USA 1997, 94:9608-13.
Eram M S, Oduaran E, Ma K: The bifunctional pyruvate decarboxylase/pyruvate ferredoxin oxidoreductase from Thermococcus guaymasensis. Archaea 2014, 2014:349-379.
Thauer R K, Kirchniawy F H, Jungermann K A: Properties and Function of the Pyruvate-Formate-Lyase Reaction in Clostridiae. Eur J Biochem 1972, 27:282-290.
Zeikus J: Metabolism of One-Carbon Compounds by Chemotrophic Anaerobes. In Advances in Microbial Physiology. Volume 24. Elsevier; 1983:215-299. [Advances in Microbial Physiology]
Amador-Noguez D, Feng X, Fan J, Roquet N, Rabitz H, Rabinowitz J D: Systems-level metabolic flux profiling elucidates a complete, bifurcated tricarboxylic acid cycle in Clostridium acetobutylicum. J Bacteriol 2010, 192:4452-61.
Cronan J E, Zhao X, Jiang Y: Function, Attachment and Synthesis of Lipoic Acid in Escherichia Coli. Volume 50. Elsevier Masson S A S; 2005.
Wigley D B, Gamblin S J, Turkenburg J P, Dodson E J, Piontek K, Muirhead H, Holbrook J J: Structure of a ternary complex of an allosteric lactate dehydrogenase from Bacillus stearothermophilus at 2.5 A resolution. J Mol Biol 1992, 223:317-35.
Mai V, Lorenz W W, Wiegel J: Transformation of Thermoanaerobacterium sp. strain JW/SL-YS485 with plasmid pIKM1 conferring kanamycin resistance. FEMS Microbiol Lett 1997, 148:163-167.
Podkaminer K K, Guss A M, Trajano H L, Hogsett D A, Lynd L R: Characterization of xylan utilization and discovery of a new endoxylanase in Thermoanaerobacterium saccharolyticum through targeted gene deletions. Appl Environ Microbiol 2012, 78:8441-7.

What is claimed is:

1. A cellulolytic microorganism comprising an exogenous adhA gene, wherein said adhA gene encodes an alcohol dehydrogenase A having a sequence that is at least 90% identical to the sequence of SEQ ID No. 2.

2. The microorganism of claim 1, wherein said microorganism is a thermophilic bacterium.

3. The microorganism of claim 1, wherein said microorganism is Clostridium thermocellum.

4. The microorganism of claim 1, wherein said microorganism is a transgenic microorganism.

5. The microorganism of claim 1, further comprising an exogenous adhE gene, wherein said adhE gene encodes an aldehyde and alcohol dehydrogenase E having a sequence that is at least 90% identical to the sequence of SEQ ID No. 1.

6. The microorganism of claim 1, wherein the sequence of the alcohol dehydrogenase A encoded by said adhA gene is at least 99% identical to the sequence of SEQ ID No. 2.

7. The microorganism of claim 5, wherein the sequence of the aldehyde and alcohol dehydrogenase E encoded by said adhE gene is identical to the sequence of SEQ ID No. 1.

8. The microorganism of claim 1, wherein the sequence of the alcohol dehydrogenase A encoded by said adhA gene is identical to the sequence of SEQ ID No. 2.

9. The microorganism of claim 1, further comprising an exogenous nfnA gene, wherein said nfnA gene encodes a protein having a sequence that is at least 90% identical to the sequence of SEQ ID No. 4.

10. The microorganism of claim 1, further comprising an exogenous nfnB gene wherein said nfnB gene encodes a protein having a sequence that is at least 90% identical to the sequence of SEQ ID No. 5.

11. The microorganism of claim 1, wherein neither exogenous nfnA nor exogenous nfnB gene is introduced into said microorganism.

12. The microorganism of claim 5, wherein said aldehyde and alcohol dehydrogenase E has a sequence of SEQ ID No. 3.

13. The microorganism of claim 1, further comprising an exogenous ferredoxin gene, wherein said exogenous ferredoxin gene encodes a protein having a sequence that is at least 90% identical to the sequence of SEQ ID No. 6.

14. The microorganism of claim 1, further comprising an exogenous pfor gene, wherein said exogenous pfor gene encodes a protein having a sequence that is at least 90% identical to the sequence of SEQ ID No. 7.

15. A cellulolytic microorganism having a modified pyruvate-to-ethanol pathway, comprising (a) an exogenous adhA gene, (b) an exogenous nfnA gene, (c) an exogenous nfnB gene, (d) an exogenous adhE gene, said exogenous adhA gene encoding an alcohol dehydrogenase A having a sequence that is at least 90% identical to the sequence of SEQ ID No. 2, said exogenous nfnA gene encoding a protein having a sequence that is at least 90% identical to the sequence of SEQ ID No. 4, said exogenous nfnB gene encoding a protein having a sequence that is at least 90% identical to the sequence of SEQ ID No. 5, said exogenous adhE gene encoding a protein having a sequence that is at least 90% identical to the sequence of SEQ ID No. 3.

16. A cellulolytic microorganism having a modified pyruvate-to-ethanol pathway, comprising (a) an exogenous adhA gene, (b) an exogenous nfnA gene, (c) an exogenous nfnB gene, (d) an exogenous ferredoxin gene, (e) an exogenous pfor gene, and (f) an exogenous adhE gene said exogenous adhA gene encoding an alcohol dehydrogenase A having a sequence that is at least 90% identical to the sequence of SEQ ID No. 2, said exogenous nfnA gene encoding a protein having a sequence that is at least 90% identical to the sequence of SEQ ID No. 4, said exogenous nfnB gene encoding a protein having a sequence that is at least 90% identical to the sequence of SEQ ID No. 5, said exogenous ferredoxin gene encoding a protein having a sequence that is at least 90% identical to the sequence of SEQ ID No. 6, said exogenous pfor gene encoding a protein having a sequence that is at least 90% identical to the sequence of SEQ ID No. 7, said exogenous adhE gene encoding a protein having a sequence that is at least 90% identical to the sequence of SEQ ID No. 3.

17. A method of producing ethanol from cellulosic biomass, comprising use of a cellulolytic microorganism having a modified pyruvate-to-ethanol pathway, said cellulolytic microorganism comprising an exogenous adhA gene, wherein said exogenous adhA gene encodes an alcohol dehydrogenase A having a sequence that is at least 90% identical to the sequence of SEQ ID No. 2.

18. The method of claim 17, wherein said microorganism further comprises either or both of nfnA gene and nfnB gene from Thermoanaerobacterium saccharolyticum.

19. The method of claim 18, wherein said microorganism further comprises the adhE gene from Thermoanaerobacterium saccharolyticum.

20. The method of claim 19, wherein said microorganism further comprises the pfor gene from Thermoanaerobacterium saccharolyticum.

21. The method of claim 19, wherein said microorganism further comprises the ferredoxin gene from Thermoanaerobacterium saccharolyticum.


SEQUENCE LISTING (33 sequences)

SEQ ID NO: 1 - protein, 859 aa, Thermoanaerobacterium saccharolyticum
MATTKTELDVQKQIDLLVSRAQEAQKKFMSYTQEQIDAIVKAMALAGVDKHVELAKMAYEETKMGVYEDKITKNLFATEYVYHDIKNEKTVGIINENIEENYMEVAEPIGVIAGVTPVTNPTSTTMFKCLISIKTRNPIIFSFHPKAIKCSIAAAKVMYEAALKAGAPEGCIGWIETPSIEATQLLMTHPGVSLILATGGAGMVKAAYSSGKPALGVGPGNVPCYIEKSANIKRAVSDLILSKTFDNGVICASEQAVIIDEEIADEVKKLMKEYGCYFLNKDEIKKLEKFAIDEQSCAMSPAVVGQPAAKIAEMAGFKVPEGTKILVAEYEGVGPKYPLSREKLSPILACYTVKDYNEGIKKCEEMTEFGGLGHSAVIHSENQNVINEFARRVRTGRLIVNSPSSQGAIGDIYNTNTPSLTLGCGSMGRNSTTDNVSVKNLLNIKRVVIRKDRMKWFKIPPKIYFESGSLQYLCKVKRKKAFIVTDPFMVKLGFVDKVTYQLDKANIEYEIFSEVEPDPSVDTVMNGVKIMNSYNPDLIIAVGGGSAIDAAKGMWLFYEYPDTEFETLRLKFADIRKRAFKFPELGKKALFIAIPTTSGTGSEVTAFAVITDKKRNIKYPLADYELTPDIAIIDPDLTKTVPPSVTADTGMDVLTHAIEAYVSVMASDYTDALAEKAIKIVFEYLPRAYKNGNDEEAREKMHNASCMAGMAFTNAFLGINHSMAHILGGKFHIPHGRANAILLPYVIRYNAEKPTKFVAFPQYEYPKAAERYAEIAKFLGLPASTVEEGVESLIEAIKNLMKELNIPLTLKDAGINKEQFEKEIEEMSDIAFNDQCTGTNPRMPLTKEIAEIYRKAYGA

SEQ ID NO: 2 - protein, 399 aa, Thermoanaerobacterium saccharolyticum
MWETKVNPSKIFELRCKNTTYFGVGSIHKIKDILENLKINGINNVIFITGKGSYKTSGAWDVVRPVLEELDLKYSLYDKVGPNPTVDMIDEAAKIGRESGAKAVIGIGGGSPIDTAKSVAVLLKYTDKNARELYKQKFIPDDAVPIIAINLTHGTGTEVDRFAVATIPEKNYKPAIAYDCLYPMFAIDDPSLMTKLDKKQTIAVTVDALNHITEAATTLVASPYSILTAKETVRLIVRYLPAAVNDPLNIVARYYLLYASALAGISFDNGLLHLTHALEHPLSAVKPEIAHGLGLGAILPAVIKAIYPATAEVLADVYSPIVPGLKGLPVEAEYVAEKVQEWLFSVGCIQKLSDFGFTKDDIPNLVKLAKTTPSLDGLLSIAPVEATESVIEKIYLKSL

SEQ ID NO: 3 - protein, 859 aa, Thermoanaerobacterium saccharolyticum
MATTKTELDVQKQIDLLVSRAQEAQKKFMSYTQEQIDAIVKAMALAGVDKHVELAKMAYEETKMGVYEDKITKNLFATEYVYHDIKNEKTVGIINENIEENYMEVAEPIGVIAGVTPVTNPTSTTMFKCLISIKTRNPIIFSFHPKAIKCSIAAAKVMYEAALKAGAPEGCIGWIETPSIEATQLLMTHPGVSLILATGGAGMVKAAYSSGKPALGVGPGNVPCYIEKSANIKRAVSDLILSKTFDNGVICASEQAVIIDEEIADEVKKLMKEYGCYFLNKDEIKKLEKFAIDEQSCAMSPAVVGQPAAKIAEMAGFKVPEGTKILVAEYEGVGPKYPLSREKLSPILACYTVKDYNEGIKKCEEMTEFGGLGHSAVIHSENQNVINEFARRVRTGRLIVNSPSSQGAIGDIYNTNTPSLTLGCGSMGRNSTTDNVSVKNLLNIKRVVIRKDRMKWFKIPPKIYFESGSLQYLCKVKRKKAFIVTDPFMVKLGFVDKVTYQLDKANIEYEIFSEVEPDPSVDTVMNGVKIMNSYNPDLIIAVGDGSAIDAAKGMWLFYEYPDTEFETLRLKFADIRKRAFKFPELGKKALFIAIPTTSGTGSEVTAFAVITDKKRNIKYPLADYELTPDIAIIDPDLTKTVPPSVTADTGMDVLTHAIEAYVSVMASDYTDALAEKAIKIVFEYLPRAYKNGNDEEAREKMHNASCMAGMAFTNAFLGINHSMAHILGGKFHIPHGRANAILLPYVIRYNAEKPTKFVAFPQYEYPKAAERYAEIAKFLGLPASTVEEGVESLIEAIKNLMKELNIPLTLKDAGINKEQFEKEIEEMSDIAFNDQCTGTNPRMPLTKEIAEIYRKAYGA

SEQ ID NO: 4 - protein, 284 aa, Thermoanaerobacterium saccharolyticum
MNEILEKKQLNPTVKMMVINAPLMAKKAKPGQFVIVRVDEKGERIPLTIADYDRNKGTITIIFQEVGMSTKKLGTLNVGDRLHDFVGPLGKPVEFSKDTKRVLAIGGGVGVAPLYPKVKMLNEMKVPVDSIIGGRSAEYVILEDEMKKVSENLYITTDDGTKGRKGFVTDVLKELIEKDNKYDEVIAIGPLIMMKMVCNITKEYNIPTMVSMNPIMIDGTGMCGGCRVTVGGETKFACVDGPAFDGLKVDFDEAMRRQNMYKDMERKVLENYEHECKLGGILNG

SEQ ID NO: 5 - protein, 463 aa, Thermoanaerobacterium saccharolyticum
MANMSLKKVPMPEQEPDQRNKNFKEVALGYEENMAVEEAERCIQCKNQPCVEGCPVHVKIPEFIKLIANRDFEGAYQKIKETNNLPAICGRVCPQESQCESVCTRGKKGEPVAIGRLERFTADWHMKNNEDKIEKPETNGRKVAVIGSGPAGLSCAGDLAKMGYDTTIFEAFHTPGGVLMYGIPEFRLPKEIVQKEIDSLKKLGVKIETNMVIGKILTIDDLFDMGYEAVFIGTGAGLPKFMNIPGENLNGVYSANEFLTRINLMKAYDFPNSPTPVKVGKKVAVVGGGNVAMDAARSAKRMGAEEVYIVYRRSEEEMPARLEEIHHAKEEGIIFKLLTNPVRIIGDESGSVKGIECVNMVLGDVDESGRRRPVEEKGSEHVIDVDTVIIAIGQSPNPLITSTTEGLEKQRWGGIIVNEETLETSRRGVFAGGDAVTGAATVILAMGAGKKAAASIHKYLSEK

SEQ ID NO: 6 - protein, 56 aa, Thermoanaerobacterium saccharolyticum
MAHIITDECISCGACAAECPVDAIHEGTGKYEVDADTCIDCGACEPVCPTGAIKAE

SEQ ID NO: 7 - protein, 1175 aa, Thermoanaerobacterium saccharolyticum
MSKVMKTMDGNTAAAHVAYAFTEVAAIYPITPSSPMAEHVDEWSAHGRKNLFGQEVKVIEMQSEAGAAGAVHGSLAAGALTTTFTASQGLLLMIPNMYKIAGELLPGVFHVSARALASHALSIFGDHQDVMACRQTGFALLASGSVQEVMDLGSVAHLAAIKGRVPFLHFFDGFRTSHEYQKIEVMDYEDLRKLLDMDAVREFKKRALNPEHPVTRGTAQNPDIYFQEREASNRYYNAVPEIVEEYMKEISKITGREYKLFNYYGAPDAERIVIAMGSVTETIEETIDYLLKKGEKVGVVKVHLYRPFSFKHFMDAIPKTVKKIAVLDRTKEAGAFGEPLYEDVRAAFYDSEMKPIIVGGRYGLGSKDTTPAQIVAVFDNLKSDTPKNNFTIGIVDDVTYTSLPVGEEIETTAEGTISCKFWGFGSDGTVGANKSAIQIIGDNTDMYAQAYFSYDSKKSGGVTISHLRFGKKPIRSTYLINNADFVACHKQAYVYNYDVLAGLKKGGTFLLNCTWKPEELDEKLPASMKRYIAKNNINFYIINAVDIAKELGLGARINMIMQSAFFKLANIIPIDEAVKHLKDAIVKSYGHKGEKIVNMNYAAVDRGIDALVKVDVPASWANAEDEAKVERNVPDFIKNIADVMNRQEGDKLPVSAFVGMEDGTFPMGTAAYEKRGIAVDVPEWQIDNCIQCNQCAYVCPHAAIRPFLLNEEEVKNAPEGFTSKKAIGKGLEGLNFRIQVSVLDCTGCGVCANTCPSKEKSLIMKPLETQLDQAKNWEYAMSLSYKENPLGTDTVKGSQFEKPLLEFSGACAGCGETPYARLVTQLFGDRMLIANATGCSSIWGGSAPSTPYTVNKDGHGPAWANSLFEDNAEFGFGMALAVKQQREKLADIVKEALELDLTQDLKNALKLWLDNFNSSEITKKTANIIVSLIQDYKTDDSKVKELLNEILDRKEYLVKKSQWIFGGDGWAYDIGFGGLDHVLASGEDVNVLVFDTEVYSNTGGQSSKATPVGAIAQFAAAGKGIGKKDLGRIAMSYGYVYVAQIAMGANQAQTIKALKEAESYPGPSLIIAYAPCINHGIKLGMGCSQIEEKKAVEAGYWHLYRYNPMLKAEGKNPFILDSKAPTASYKEFIMGEVRYSSLAKTFPERAEALFEKAEELAKEKYETYKKLAEQN

SEQ ID NO: 8 - DNA, 18 bp, Artificial Sequence
  Synthetic
taaaccgctaaggcatga

SEQ ID NO: 9 - DNA, 19 bp, Artificial Sequence
  Synthetic
ctatctgcatcgtcttttc

SEQ ID NO: 10 - DNA, 18 bp, Artificial Sequence
  Synthetic
agttaggatgttggcaga

SEQ ID NO: 11 - DNA, 18 bp, Artificial Sequence
  Synthetic
aaagagggcatacaagga

SEQ ID NO: 12 - DNA, 19 bp, Artificial Sequence
  Synthetic
tgcaggtcgataaacccag

SEQ ID NO: 13 - DNA, 28 bp, Artificial Sequence
  Synthetic
gaattccctttagtaacgtgtaactttc

SEQ ID NO: 14 - DNA, 19 bp, Artificial Sequence
  Synthetic
cattaatgaatcggccaac

SEQ ID NO: 15 - DNA, 18 bp, Artificial Sequence
  Synthetic
ctcgtgatacgcctattt

SEQ ID NO: 16 - DNA, 18 bp, Artificial Sequence
  Synthetic
gctgtggcaacttaacaa

SEQ ID NO: 17 - DNA, 18 bp, Artificial Sequence
  Synthetic
ctcatatcatccgctcct

SEQ ID NO: 18 - DNA, 19 bp, Artificial Sequence
  Synthetic
gttgttgttttggcttagg

SEQ ID NO: 19 - DNA, 18 bp, Artificial Sequence
  Synthetic
aggctttcattcagtacg

SEQ ID NO: 20 - DNA, 18 bp, Artificial Sequence
  Synthetic
cgtgccttttgaccttcc

SEQ ID NO: 21 - DNA, 18 bp, Artificial Sequence
  Synthetic
ctgctgtctcgtcctatt

SEQ ID NO: 22 - DNA, 18 bp, Artificial Sequence
  Synthetic
ccaatataccaccagcca

SEQ ID NO: 23 - DNA, 19 bp, Artificial Sequence
  Synthetic
gaatttaggaaaaccgcca

SEQ ID NO: 24 - DNA, 19 bp, Artificial Sequence
  Synthetic
atccctctgtgtctttatc

SEQ ID NO: 25 - DNA, 18 bp, Artificial Sequence
  Synthetic
tggttgtgggtgtttatg

SEQ ID NO: 26 - DNA, 20 bp, Artificial Sequence
  Synthetic
gaagccttagtgcgaagtgg

SEQ ID NO: 27 - DNA, 20 bp, Artificial Sequence
  Synthetic
gaagtccaacatgtgcatcg

SEQ ID NO: 28 - DNA, 20 bp, Artificial Sequence
  Synthetic
atcaagcttggaatgggttg

SEQ ID NO: 29 - DNA, 20 bp, Artificial Sequence
  Synthetic
gctgttggagcctttgagtc

SEQ ID NO: 30 - DNA, 20 bp, Artificial Sequence
  Synthetic
ctatagcatcgcctgctgtg

SEQ ID NO: 31 - DNA, 20 bp, Artificial Sequence
  Synthetic
tcgataccgccgtttatagc

SEQ ID NO: 32 - DNA, 20 bp, Artificial Sequence
  Synthetic
attgccataaccctgacaca

SEQ ID NO: 33 - DNA, 20 bp, Artificial Sequence
  Synthetic
taggctctccacctgtcagc